(19) United States
(12) Patent Application Publication
(10) Pub. No.: US 2024/0124891 A1
(43) Pub. Date: Apr. 18, 2024

(54) CRISPR/CAS9-MEDIATED MEANS AND METHODS FOR CELL REPROGRAMMING

(71) Applicant: Helmholtz Zentrum München - Deutsches Forschungszentrum Für Gesundheit Und Umwelt (GmbH), Neuherberg (DE)

(72) Inventors: Wolfgang WURST, Muenchen (DE); Florian GIESERT, Muenchen (DE); Jessica GIEHRL-SCHWAB, Muenchen (DE); Benedict RAUSER, Muenchen (DE)

(21) Appl. No.: 18/277,558

(22) PCT Filed: Feb. 17, 2022
(86) PCT No.: PCT/EP2022/053926

§ 371 Date: Aug. 16, 2023

(30) Foreign Application Priority Data
Feb. 17, 2021 (LU) LU102570

(51) Int. Cl. C12N 15/86; A61P 25/16; C12N 9/22; C12N 15/11

(52) U.S. Cl. CPC C12N 15/86; A61P 25/16; C12N 9/22; C12N 15/11; C07K 2319/92; C12N 2310/20; C12N 2750/14143; C12N 2800/40; C12N 2840/445

(57) ABSTRACT

The present invention relates to CRISPR-mediated means and methods, e.g., for adjustably induction of multiple gene expression and subsequent cell reprogramming. Particularly, the CRISPR-mediated means and methods of the present invention relate to conversion of endogenous glial cells into GABAergic neurons representing an effective method for cell reprogramming.

This application contains a Sequence Listing in computer readable form, which is incorporated herein by reference.

### TECHNICAL FIELD

The present invention relates to CRISPR/Cas9-mediated means and methods that can, for example, be used for adjustably induction of multiple gene expression and subsequent cell reprogramming. The CRISPR-mediated means and methods of the present invention further relate to conversion of endogenous glial cells into neurons by activation of specific endogenous genes representing an effective method for cell reprogramming. In the course of the present invention a knock-in mouse line carrying a dual dCas9 trans-activator system (referred to as dCAM) has been developed allowing for the conditional in vivo activation of one or more endogenous genes. Spatial and cell type specificity can be achieved by stereotactic injections of the gene-specific gRNAs into distinct regions of a cell-type specific Cre-expressing mouse line. The present invention further relates to an AAV-based system comprising intein-split-dCas9 polypeptides in combination with activators and specific sgRNAs (referred to as AAV-dCAS). This activation tool is independent of a transgenic dCas9 trans-activator system and can be applied as an autonomous therapeutic tool upon minor adaptions. The fields of application are in general diseases were ectopic expression or overexpression of endogenous genes can contribute to the amelioration of symptoms to the point of completely restoring the disease either solei by single or multiple gene induction or by subsequent cellular reprogramming/trans-differentiation. Such means and methods of the present invention (dCAM and AAV-dCAS) were shown to be successful in reprogramming murine striatal astrocytes into induced neurons by activation of the endogenous expression factors Ascl1, Lmx1a and Nr4a2. The majority of these neurons, which were shown to exhibit a GABAergic identity in single cell transcriptome analysis, functionally integrate into striatal circuits as evidenced by the alleviation of voluntary motor behaviour aspects in the 6-OHDA toxin-induced Parkinson's disease model. Accordingly, the present invention further relates to novel therapies for Parkinson's disease, which go beyond mere restoration of dopamine levels and therefore define new patient treatment groups. Furthermore, means and methods of the present invention (e.g., AAV-dCAS) can enable clinical therapies for Parkinson's disease by reprogramming striatal astrocytes.

### BACKGROUND OF THE INVENTION

Parkinson's disease is the second most common neurodegenerative disorder, characterized by the degeneration of nigrostriatal dopaminergic neurons in the substantia nigra pars compacta (SNpc), leading to specific motor symptoms like tremor, bradykinesia and rigidity. Current treatments focus on symptomatic disease management, either by pharmacological restoration of dopamine levels or electrophysiological pace making of downstream nuclei, which initially ameliorates the motor symptoms. Alternative therapy options, aiming to replace lost neurons, have been explored with mixed beneficial outcome for the patients, partly due to the lack of appropriate standardized foetal tissue or alternative cell source.

Accordingly, although cell replacement strategies of dopaminergic neurons have been applied in clinical studies of Parkinson's disease with variable success, further improved medications and methods for cell replacement are still needed. To further develop this approach, a more efficient genetic tool to adjustably induce multiple genes and deliver complex gene induction systems in vivo is needed.

### SUMMARY OF THE INVENTION

The present invention relates to a plurality of separate adeno-associated viruses (AAVs) comprising: (i) a first AAV comprising a first nucleic acid encoding a first portion of a Cas9 protein devoid of endonuclease activity; (ii) a second AAV comprising a second nucleic acid encoding a second portion of a Cas9 protein devoid of endonuclease activity; (iii) a third AAV comprising a third nucleic acid encoding at least one polypeptide having a trans-activating activity (e.g., activating transcription) and capable of binding to and/or associating with an at least one guide RNA (gRNA) and/or said first and/or second portion of Cas9 protein, wherein said third nucleic acid further encoding an at least one guide RNA (gRNA) comprising at least one aptamer capable of binding at least one MS2 coactivator protein, preferably said third nucleic acid encoding a synergistic activation mediator (SAM) complex and at least one guide RNA (gRNA) comprising at least one aptamer capable of binding at least one MS2 coactivator protein, most preferably comprising at least two aptamers, each capable of binding at least two MS2 coactivator proteins; (iv) optionally, a fourth AAV comprising a fourth nucleic acid encoding a reporter polypeptide, preferably said reporter polypeptide is a fluorescent protein, further preferably said fluorescent protein is a green fluorescence protein; wherein said first portion of said Cas9 protein devoid of endonuclease activity and said second portion of said Cas9 protein devoid of endonuclease activity, when joined together, form a Cas9 protein devoid of endonuclease activity, preferably said formed Cas9 protein is capable of binding DNA..

Accordingly, the present application satisfies this need by the provision of means and methods for cell reprogramming described herein below, characterized in the claims and illustrated by the appended Examples and Figures.

### OVERVIEW OF THE SEQUENCE LISTING

As described herein references can be made to UniProtKB Accession Numbers (http://www.uniprot.org/, e.g., as available in UniProtKB release 2020_06 published Dec. 2, 2020).

As described herein references can be made to GenBank Accession Numbers (https://www.ncbi.nlm.nih.gov/genbank/release/241/), e.g., available in Release 241: of Dec. 15, 2020.

SEQ ID NO: 1 is the nucleic acid sequence encoding N-dCas9_N-Intein construct.

SEQ ID NO: 2 is the amino acid sequence of the N-dCas9_N-Intein construct.

SEQ ID NO: 3 is the nucleic acid sequence encoding C-dCas9_VP64_C-Intein construct.

SEQ ID NO: 4 is the amino acid sequence of the C-dCas9_VP64_C-Intein construct.

SEQ ID NO: 5 is the nucleic acid sequence encoding CBh_flexed-GFP construct.

SEQ ID NO: 6 is the amino acid sequence of the CBh_flexed-GFP construct.

SEQ ID NO: 7 is the nucleic acid sequence encoding HA_SpCas9 (10A, H840A)_VPR construct.

SEQ ID NO: 8 is the amino acid sequence of the HA_SpCas9 (D10A,H840A)_VPR construct.

SEQ ID NO: 9 is the nucleic acid sequence encoding an exemplary N-Split-Intein.

SEQ ID NO: 10 is the amino acid sequence of an exemplary N-Split-Intein.

SEQ ID NO: 11 is the nucleic acid sequence encoding an exemplary C-Split-Intein.

SEQ ID NO: 12 is the amino acid sequence of an exemplary C-Split-lntein.

SEQ ID NO: 13 is the nucleic acid sequence encoding the MS2.

SEQ ID NO: 14 is the amino acid sequence of the MS2.

SEQ ID NO: 15 is the nucleic acid sequence encoding the MS2-p65 construct.

SEQ ID NO: 16 is the amino acid sequence of the MS2-p65 construct.

SEQ ID NO: 17 is the nucleic acid sequence encoding the MS2-p65-HSF1 construct.

SEQ ID NO: 18 is the amino acid sequence of the MS2-p65-HSF1 construct.

SEQ ID NO: 19 is the nucleic acid sequence encoding the VP16.

SEQ ID NO: 20 is the amino acid sequence of the VP16.

SEQ ID NO: 21 is the nucleic acid sequence encoding the dCas9-SAM-P2A-VPR construct.

SEQ ID NO: 22 is the nucleic acid sequence encoding the AAV-ALN-flexGFP construct.

SEQ ID NO: 23 is the nucleic acid sequence encoding the AAV-N-flex-dCas9aa1-573-N-intein construct.

SEQ ID NO: 24 is the nucleic acid sequence encoding the AAV-C-dCas9aa574-1368-VP64-C-intein construct.

SEQ ID NO: 25 is the nucleic acid sequence encoding the AAV-Lmx1a-Nr4a2-SAM construct.

SEQ ID NO: 26 is the nucleic acid sequence encoding the AAV-flexGFP construct.

SEQ ID NO: 27: is the amino acid sequence of the Streptococcus pyogenes serotype M1 derived CRISPR-associated endonuclease Cas9/Csn1 having UniProtKB Accession Number Q99ZW2.

SEQ ID NOs: 28-45 exemplary DNA binding sites' sequences.

### BRIEF DESCRIPTION OF THE DRAWINGS

FIG. 1: Rosa26 knock-in dCas9 Activator Mouse (dCAM) based reprogramming of astrocytes. a, Knock-in of a conditional dCas9-SAM-P2A-VPR expression cassette into the Gt(ROSA)26Sor locus enables flexible multiplexed endogenous gene activation in vitro and in vivo. Cassette is composed of LoxP-puro-stop-LoxP followed by the SAM activator (flanked by FRT sites), a P2A sequence and the dCas9-VPR. Expression is driven by the strong, ubiquitous CAG promoter. dCAM x Gfap-Cre mice enable astrocyte-specific dCas9 and activator expression. For in vivo activation an AAV containing 6 sgRNAs and a reporter gene can be applied. AAVs contain sgRNAs, whose expression is driven by the different Pol III promoters (H1, hU6, mU6 and 7SK), and the marker gene FLEx-GFP, respectively split-FLEx-GFP, is expressed by the CBh promoter and also delivered by AAVs. b, Multiplexed activation of Ascl1, Lmx1a, NeuroD1 (Ascl1 28±11, Lmx1a 27±7, NeuroD1 250 ±70) and of Ascl1, Lmx1a, Nr4a2 (Ascl1 6±1, Lmx1a 23 ±8, Nr4a27±0) in primary astrocytic cultures. n=2-3, technical replicates, one representative run is shown, additional data in supplement. Activation levels are depicted as fold change between cells transfected with and without sgRNAs. All levels were normalized to β-Actin. Error bars represent mean ±SD between technical replicates. c, Toxin-induced neurodegeneration model (6-OHDA) is applied to induce Parkinson's disease. Two weeks after the induction of the neurodegeneration in the medium forebrain bundle the AAV is injected into the dorsal striatum, after a five weeks period the first animals are analyzed. After another eight weeks period animals undergo another analysis including behavior tests, electrophysiological measurements and immunohistochemistry. d, Photomicrographs showing GFP⁺/GFAP⁺ cells 13 wpi. Arrows indicating GFP⁺/GFAP⁻ cells. e, Quantification of GFAP⁺/GFP₊ cells. GFP 92.3±0.42%, ALNe-218 78.45±5.63% and ALN 66.57±2.35%. GFP vs. ALNe-218 P=0.0064, GFP vs. ALN P=0.0002 and ALN vs. ALNe-218 P=0.0127. Multiple comparison ANOVA F(2,7)=32.06. f, Photomicrographs showing GFP⁺/NeuN⁺ neurons 13 wpi. Arrow heads indicating GFP⁺/NeuN⁺ cells. g Quantification of NeuN⁺/GFP +cells. GFP 5.2±0.26%, ALNe-218 13.17±1.36% and ALN 17.87±0.50%. GFP vs. ALNe-218 P<0.0001, GFP vs. ALN P<0.0001 and ALN vs. ALNe-218 P=0.0012. Multiple comparison ANOVA F(2,6)=170.3. Scale bar indicates 50 μm. Tukey's multiple comparisons test * P<0.05, **P<0.01, ***P<0.001, ****P<0.0001. Error bars represent mean ±SD. Abbreviations: Puro-puromycin resistance, SAM-synergistic activation mediator (MS2: MS2 bacteriophage coat protein, p65: p65 subunit of human NF-KB, HSF1: Heat shock factor 1), P2A-2A self-cleaving peptide, dCas9-deadCas9 (nuclease-deficient), VPR-VP64: 4× VP16 herpes simplex virus protein vmw65, p65, Rta: Regulator of transcriptional activation, CAG-CMV early enhancer/chicken β actin promoter, CBh-chicken β-actin hybrid promoter. SgRNA expression is driven by the different Pol III promoters (H1, hU6, mU6 and 7SK).

FIG. 2: AAV-split-dCas9 Activator System (AAV-dCAS) based reprogramming of astrocytes. a, dCas9 is separated into a N- and a C-terminal part (AAV-N-dCas9^{aa1-573} -N-intein and AAV-C-dCas9^{aa574-1368} -VP64-C-intein), both portions are fused to the corresponding intein-moieties. Upon co-expression intein-mediated trans-splicing leads to a reconstitution of the protein. b, RT-qPCR analysis of Ascl1 induction for the comparison of activation capacity of full-length versus split-dCas9 in Neuro2A cells. Data in fold change normalized to non-activated control: dCas9 6116÷847.3, split-dCas9 4415±748.8, n=3, activation levels are depicted as fold change between cells transfected with to without sgRNAs. c, Immunocytochemistry analysis of reprogrammed cells 16 days after lentiviral transduction revealed successful in vitro reprogramming of astrocytes into neurons using the CRISPRa system. Scale bar indicates 50 μm. d, Upon splitting of the large dCas9 gene into two parts, the system can be packed into AAVs. To ensure cell type specificity upon Cre expression the N-dCas9 and the GFP are inverted and flanked by two different LoxP sites (LoxX and Lox511). dCas9 is delivered by two AAVs, a third AAV is needed for the delivery of the SAM activator. A forth virus contains the reporter gene, while sgRNAs are distributed between the vectors. e, Multiplexed activation of Ascl1, Lmx1a, NeuroD1 (Ascl1 98±23, Lmx1a 99±9, NeuroD1 1452±109); Ascl1, Lmx1a, Nr4a2 (Ascl1 55±17, Lmx1a 92±27, Nr4a2 79±8) and Ascl1, Lmx1a, Nr4a2, PITX3, FoxA2 (Ascl1 183±16, Lmx1a 228±13, Nr4a2 122±12, PITX3 220±6, FoxA2 36±12) in primary astrocytic cells. n=2-3, technical replicates, one representative run is shown, additional data in supplement. Activation levels are depicted as fold change between cells transfected with and without sgRNAs. All levels were normalized to β-Actin. Error bars represent mean ±SD between technical replicates. f, Photomicrographs showing GFP⁺/GFAP⁺ cells 13 wpi. Arrows indicating GFP⁺/GFAP⁻ cells. g, Quantification GFAP⁺/GFP₊ cells. GFP 93.0±1.85%, ALNe-218 76.23±3.27% and ALN 48.0±6.65%. GFP vs. ALNe-218 P=0.0083, GFP vs. ALN P<0.0001 and ALN vs. ALNe-218 P=0.0006. Multiple comparison ANOVA F(2,6)=79.76. h, Photomicrographs showing GFP⁺/NeuN⁺ neurons 13 wpi. Arrow heads indicating GFP⁺/NeuN⁺ cells. i, Quantification NeuN⁺/GFP⁺ cells. GFP 5.6±2.35%, ALNe-218 11.67±0.35% and ALN 25.47±6.85%. GFP vs. ALN P=0.0008, ALN vs. ALNe-218 P=0.0092. Multiple comparison ANOVA F(2,7)=21.74. j, Confocal images showing co-localization of GFP and glutamic acid decarboxylase, a marker specific for GABAergic neurons. Scale bar indicates 50 μm. Error bars represent mean ±SD. Tukey's multiple comparisons test * P<0.05, **P<0.01, ***P<0.001, ****P<0.0001. Abbreviations: dN-Cas9-N-terminal dCas9-residues 1-573, N-intein-N-terminal part of DNA polymerase III subunit alpha, dC-Cas9-C-terminal dCas9 residues 574-1368, C-intein-C-terminal part of DNA polymerase Ill subunit alpha, VP64-4× VP16 herpes simplex virus protein vmw65, p65-p65 subunit of human NF-KB, HSF1-heat shock factor 1, MS2-MS2 bacteriophage coat protein, PAM—protospacer adjacent motif, TSS—transcriptional start site, OE—overexpression, CBh-chicken β-actin hybrid promoter. SgRNA expression is driven by the different Pol III promoters (H1, hU6, mU6 and 7SK).

FIG. 3: Analysis of striatal tissue from ALN reprogrammed dCAM mice by single cell RNA-seq. a, a, Scheme depicting experimental preparation of cells of 13 wpi mice striatal regions (n=2, one technical replicate). Papain dissociated cells are prepared for scRNA-seq using droplet-based separation and barcoding. Uniform Manifold Approximation and Projection (UMAP) visualization of QC-selected cells for GFP and ALN (n=3,899). Color labeling highlights nine main cell groups based on Leiden clustering and identification based on marker genes. Rectangle highlights astrocytic and neuronal cell clusters. 4,273 highly variable genes (HVG) were detected. b, Subclustering of 1,110 cells identified four groups of astrocytic and neuronal identity. Layout is based on UMAP visualization presented in a. Clustering of markers genes selected based on expression levels between clusters. Expression Z-scores are hierarchically clustered by rows. c, GFP control and ALN reprogrammed cells selected from the neuronal and astrocytic clusters are visualized based on detection of GFP (red cells), marker gene Ascl1, Myt1I and Gad1/Gad2 (Gad1/2) (blue cells), and the co-detection of both (yellow cells).

FIG. 4: Electrophysiological characterization of induced neurons and motor behaviour analysis 13 wpi. a, Firing pattern of a neuron reprogrammed by the endogenous activation of Ascl1, Lmx1a, NeuroD1 and expression of miRNA218 (ALNe-218). 10/10 cells showed electrophysiological properties of immature neuron/glia-like cells (i.e. lack of APs and a relatively low Rᵢₙ). b, Firing pattern of a neuron reprogrammed by the endogenous activation of Ascl1, Lmxia, Nr4a2 (ALN). 14/15 cells exhibit action potentials, 1/15 showed electrophysiological properties of immature neuron/glia-like cells (i.e. lack of APs and a relatively low Rᵢₙ). Bottom right: example of spontaneous synaptic events recorded from a different reprogrammed neuron, therefore showing the integration within striatal neuronal network.c, Left panel-the resting membrane potential (Vₘ in mV) is similar between different reprogramming conditions. Right panel-Input resistance (Rᵢₙ in mΩ) is significantly different between the different conditions (p=0.002, Kruskall-Wallis test). The input resistance of cells measured in the ALNe-218 condition are similar to immature neurons/glia-like cells, whereas ALN reprogrammed cells exhibit an input resistance within the range of endogenous neurons. Kruskall-Wallis test **P<0.01. Error bars represent mean ±SEM. d, Gait analysis using the CatWalk XT system. Average speed of tread. Data in cm/s: Naïve 43.52±5.48, 6-OH DA 27.66±2.78, dCAM: GFP 33.22±10.30, ALNe-218 34.09±6.94, ALN 38.76±11.45, AAV-dCAS: GFP 25.14±4.10, ALNe-218 28.70±5.44, ALN 34.31±4.87. Significant decrease in speed due to 6-OHDA lesion (13 wpi). Naïve vs. 6-OHDA P=0.0063. AAV-dCAS: GFP vs. ALN P=0.015 multiple comparison ANOVA F(2,17)=12.81. e, Stride length of front paws. Data in cm. Naïve 8.32±0.46, 6-OHDA 6.83±0.66, dCAM: GFP 7.39±0.67, ALNe-218 7.58±0.66, ALN 8.02±0.99. AAV-dCAS: GFP 6.73±0.47, ALNe-218 7.12±0.43, ALN 7.56±0.43. Naïve vs. 6-OHDA P=0.0164. AAV-dCAS: GFP vs. ALN P=0.005, ALN vs. ALNe-218 P=0.0042, multiple comparison ANOVA F(4,22)=9.9. f, Duty cycle of left front paw. Data in %: Naïve 48.24±1.86, 6-OHDA 54.42±2.15, dCAM: GFP 52.29±2.29, ALNe-218 52.57±2.10, ALN 49.55±1.70, AAV-dCAS: GFP 53.59±3.40, ALNe-218 53.42±2.44, ALN 50.93±1.67. Naïve vs. 6-OHDA P=0.0096. dCAM: GFP vs. ALN P=0.036 and ALN vs. ALNe-218 P=0.0252, multiple comparison ANOVA F(2,20)=5.199. g, Amphetamine-induced rotation analysis. Change in rotational behavior in lesioned animals upon treatment with dopamine releaser substance. Net rotation =ipsilateral rotation-contralateral rotation. Naïve 32.0±11.42, 6-OHDA 252.0±128.2, dCAM: GFP 244.9±31.16, ALNe-218 191.4±36.68, ALN 293.8±40.84, dCAS: GFP 233.8±67.21, ALNe-218 210.6±51.94, ALN 192.9±72.66. Naïve vs. 6-OHDA P=0.09. Naïve vs. 6-OHDA unpaired t-test (two-tailed) * P<0.05, ** P<0.01. GFP vs ALN, GFP vs ALNe-218 and ALN vs ALNe-218 Tukey's multiple comparisons test * P<0.05, ** P<0.01. CatWalk error bars represent mean ±SD. Rotation analysis error bars represent mean ±SEM.

FIG. 5: Design and evaluation of the dCAM line. a, The LoxP-flanked puro-stop cassette ensures highly specific knock-in expression. Western blot analysis of targeting construct in Neuro2A cells. Left blot -Test of the P2A sequence for appropriate cleavage. Antibody binds 5″part of the P2A, SAM-5′-P2A runs at 55 kDa. No fusion products observable. Right blot-Test of the puro-stop-cassette. Without Cre no Cas9 protein visible b, Variable activation levels can be achieved, as the SAM activator is flanked by FRT sites and can be removed. c, Rosa26 knock-in design, homology arms are used 5″arm 1 kb and 3″arm 4 kb long. Southern blot analysis of the founder animals. EcoRV is used for gDNA digest, with one band at 11.5 kb-wild type band and one at 8.7 kb-knock-in band, which should occur dependent on the probe. Mouse number 3 shows double band and is used for further breeding. Genotyping PCR of F1 generation using Cas9 F and Cas9 R primers, 4 (No. 3, 4, 6, 8) out of 10 animals show knock-in. Western blot from primary astrocytes of the dCAM x GFAP-cre line. dCas9 is only detected when Cre was expressed. d, For in vivo activation an AAV containing 6 sgRNAs and a reporter gene can be applied. e, If more than 6 sgRNAs shall be used for in vivo activation two AAVs containing 12 sgRNAs or 6 sgRNAs and a miRNA expression cassette can be applied with a split-reporter gene. AAVs contain sgRNAs, whose expression is driven by the different Pol 111 promoters (H1, hU6, mU6 and 7SK), and the marker gene FLEx-GFP, respectively split-FLEx-GFP, is expressed by the CBh promoter and also delivered by AAVs. Abbreviations: Puro-puromycin resistance, SAM -synergistic activation mediator (MS2: MS2 bacteriophage coat protein, p65: p65 subunit of human NF-KB, HSF1: Heat shock factor 1), P2A-2A self-cleaving peptide, dCas9-deadCas9 (nuclease-deficient), VPR-VP64: 4× VP16 herpes simplex virus protein vmw65, p65, Rta: Regulator of transcriptional activation, CAG-CMV early enhancer/chicken β actin promoter.

FIG. 6: Evaluation of dCAM x Gfap-Cre primary astrocytes for the activation capacity. a, Multiplexed activation of Ascl1, Lmx1a and Nr4a2. (left (1): Ascl1 6±3, Lmx1a 28±12, Nr4a2 10±3, right (2): Ascl1 10±7, Lmx1a 22±3, Nr4a2 6±1) b, Multiplexed activation of Ascl1, Lmx1a and NeuroD1. (Ascl1 31±19, Lmx1a 30±23, NeuroD1 206±134). Activation levels are depicted as fold change between cells transfected with and without sgRNAs. All levels were normalized to β-Actin. Error bars represent mean ±SD between technical replicates.

FIG. 7: Evaluation of 6-ODHA induced lesion. a, b, Immunohistochemistry in an animal 14 days after the 6-OHDA injection into the medium forebrain bundle. a, Staining of dopaminergic lesion using the marker tyrosine hydroxylase (TH). b, Staining with the astrocytic marker GFAP to assess the reactive gliosis. c, Reactive gliosis was assessed via the signal intensity of GFAP stained striata. Naïve, 6 days post lesion (dpl) and 14 dpl animals were analyzed. Per condition data was collected from two animals, from each animal ten images were analyzed, randomly taken in striatal regions. Ipsilateral: Naïve 19.7±1.8, 6 dpl 28.7±6.7, 14 dpl 24.9±6.0, contralateral: Naïve 14.3±4.4, 6 dpl 14.4±2.0, 14 dpl 13.3 ±1.9.

FIG. 8: Total amount of GFP+ cells in vivo in dCAM x Gfap-Cre mice injected with FLEx-GFP reporter. GFP⁺ cells in the ipsilateral dorsal striatum of one slide after five weeks of injection. No significant difference could be observed between the different reprogramming conditions and the GFP control. GFP 892.0±85.4, ALNe-218 652.7 ±193.6, ALN 993.3±106.6. Error bars represent mean ±SD.

FIG. 9: In vivo reprogramming 5 weeks after AAV injection in dCAM x Gfap-Cre mice. a, Photomicrographs showing GFP⁺/Gfap⁺ double positive cells 5 wpi. Arrows indicating GFP⁺/Gfap⁻ cells. b, Quantification GFAP⁺/GFP₊ cells. GFP 97.13±0.45%, ALNe-218 79.33±6.05%, and ALN 86.70±1.90%. GFP vs. ALNe-218 P=0.0025, GFP vs. ALN P=0.03. Multiple comparison ANOVA F(2,6)=17.78. c, Photomicrographs showing GFP⁺/NeuN^{+ neurons} 5 wpi. Arrow heads indicating GFP⁺/NeuN⁺ cells. d, Quantification NeuN⁺/GFP⁺ cells. GFP 3.9±0.53%, ALNe-218 15.67±0.96% and ALN 14.77±3.09%. GFP vs. ALNe-218 P=0.0007, GFP vs. ALN P=0.001. Multiple comparison ANOVA F(2,6)=35-85. Scale bar indicates 50 μm. Error bars represent mean ±SD. Tukey's multiple comparisons test * P<0.05, **P<0.01, ***P<0.001, ****P<0.0001.

FIG. 10: Activation of endogenous genes using the dCas9—VPR and the SAM activator system in Neuro2A cells. a, Activation of Ascl1. (VPR 82±21, SAM 3158±10, SAM&VPR 10493±432) b, Activation of Ngn2. (VPR 355±17, SAM 2290±476, SAM&VPR 2422±195) c, Activation of MyoD1. (VPR 264±91, SAM 6047±517, SAM&VPR 6285±669) d, Activation of Pou5F1. (VPR 3682±1003, SAM 25041±2507, SAM&VPR 20534±4521). Each gene was activated by two sgRNAs targeting the 200 bp prior to the transcriptional start side. Activation levels are depicted as fold change between cells transfected with and without sgRNAs. All levels were normalized to β-Actin. n=1, with three technical replicates. Error bars represent mean ±SD between technical replicates.

FIG. 11: Evaluation of AAV-dCAS in vitro. a, Western blot analysis evaluating the FLEx-N-dCas9 system in Neuro2A cells, using a C-Cas9 antibody. b, Western blot analysis evaluating the split-dCas9 system in Neuro2A cells, left blot-N-Cas9 antibody, right blot-C-Cas9 antibody. Correct fusion of the split-dCas9 parts at 175 kDa. c, Immunocytochemistry analysis on primary astrocytic cultures. Activation of Ascl1, Lmx1a and Nr4a2. Upper lane-Transfection of dCas9-activators without sgRNAs. Lower lane -Transfection of dCas9-activators with sgRNAs. Red channel staining for the respective protein. Scale bars indicate 20 μm. d, RT-qPCR levels. Multiplexed activation of Ascl1, Lmx1a, Nr4a2 (Ascl1 103±19, Lmx1a 91±62, Nr4a2 129±16) and Ascl1, Lmx1a, and NeuroD1 (left (1): Ascl1 100±61, Lmx1a 14±7.5, NeuroD1 1542±352, right (2): Ascl1 73±6, Lmx1a 12±3, NeuroD1 1160±142) and Ascl1, Lmx1a, Nr4a2, PITX3, FoxA2 (Ascl1 167 ±30, Lmx1a 215±18, Nr4a2 107±10, PITX3 195±19, FoxA2 32±8) in primary astrocytic cultures. Activation levels are depicted as fold change between cells transfected with and without sgRNAs. All levels were normalized to β-Actin. Error bars represent mean ±SD between technical replicates

FIG. 12: in vivo reprogramming 5 wpi-AAV-dCAS reprogramming in Gfap-Cre mice. a, Photomicrographs showing GFP⁺/GFAP⁺ cells 5 wpi. Arrows indicating GFP⁺/GFAP⁻ cells. b, Quantification GFAP⁺/GFP₊ cells. GFP 95.37±0.40%, ALNe-218 80.33±1.75% and ALN 84.10±4.16%. GFP vs. ALNe-218 P=0.001, GFP vs. ALN P=0.0045. Multiple comparison ANOVA F(2,6)=26.85. c, Photomicrographs showing GFP⁺/NeuN^{+ neurons} 5 wpi. Arrow heads indicating GFP⁺/NeuN⁺ cells. d, Quantification NeuN⁺/GFP⁺ cells. GFP 4.23 ±1.55%, ALNe-218 14.10±0.89%, ALN 14.67±1.21%. GFP vs. ALNe-218 P=0.0002, GFP vs. ALN P=0.001. Multiple comparison ANOVA F(2,6)=66.67. Scale bar indicates 50 μm. Error bars represent mean ±SD. Tukey's multiple comparisons test * P<0.05, **P<0.01, ***P<0.001, ****P<0.0001.

FIG. 13: Neurotransmitter identities of reprogrammed neurons using AAV-dCAS. a, b, Confocal images showing co-localization of GFP and markers specific for neurotransmitter subtype neurons. a, Tyrosine hydroxylase-dopaminergic neurons b, Vesicular glutamate transporter 1-glutamatergic neurons. Scale bars indicate 50 μm.

FIG. 14: DARPP32 staining and quantification 13 wpi. a, b, Evaluation DARPP32 staining in dCAM model. a, Confocal images showing co-localization of GFP and DARPP32. b, Quantification DARPP32⁺/GFP⁺ cells. GFP 4.3±0.5%, ALNe-218 4.76±0.38% and ALN 5.67±0.49%. GFP vs. ALN P=0.023. Multiple comparison ANOVA F(2,6)=7.078. c, d, Evaluation DARPP32 staining in AAV-dCAS model. c, Confocal images showing co-localization of GFP and DARPP32. d, Quantification DARPP32⁺/GFP⁺ cells. GFP 3.4±0.1%, ALNe-218 3.97±0.99% and ALN 6.43±0.42%. GFP vs. ALN P=0.0024, ALN vs. ALNe-218 P=0.0067. Multiple comparison ANOVA F(2,6)=20.24. Scale bars indicate 50 μm. Error bars represent mean ±SD. Tukey's multiple comparisons test * P<0.05, **P<0.01, ***P<0.001.

FIG. 15: Phenotypical identities of reprogrammed neurons using AAV-dCAS. a-d, Confocal images showing co-localization of GFP and the interneuron markers: a, parvalbumin, b, calretinin, c, neuropeptide Y and d, choline acetyl transferase. Scale bars indicate 50 μm.

FIG. 16: Quality control of single cell RNA-seq at 13 wpi of AAVs in dCAM x GFAP-Cre mice striatal tissue. a, Number of genes (y-axis) versus count depth (x-axis) per cell. Color highlights fraction of mitochondrial reads. Quality control thresholds of 800 and 250 for number of genes and minimum cell depth are defined, respectively, obtaining 3,899 cells. b, Distributions of count depth for all cells. Inset shows count depth distribution from for all cells with fewer than 4000 counts. The count depth threshold of 800 is shown as a red, vertical line. c, Distribution of number of genes detected per cell. Red line indicates thresholds as in a.

FIG. 17: Rank_genes_groups plots from Scanpy showing the top 25 marker genes using a t-test between log-normalized expression values. a, Top 25 marker genes ranked by their using t-test statistic when comparing normalized cell counts between annotated group against all other groups. b, Same as in a but using four astrocytic-neuronal sub-clusters (n=1,110 cells). Colors as in FIG. 4 a, b.

FIG. 18: scRNA-seq analysis 13 wpi in dCAM x GFAP-Cre mice. a, Pearson's correlation coefficient of top-10 marker gene expression levels of cells in astrocytic subgroups (n=643) and in neuronal cell subgroups (n=467). b, Counts for GFP +cells (red), markers Cre, Nr4a2, and Lmx1a (blue) and co-detection of cell with both markers (yellow) in GFP control and ALN reprogramming. c, Same as in b, but showing Gad1, Gad2, Th and Slc17a 7 as markers genes.

FIG. 19: Electrophysiological measurements 5 wpi to induce the factors Ascl1, Lmx1a, Nr4a2 in the AAV-dCAS setting. Firing pattern of induced neurons 5 weeks after sgRNA injection. Neurons exhibit electrophysiological properties of immature neurons (cell 1 exhibited one action potential) respectively of glial cells (cell 2 and 3).

FIG. 20: Motor behavior analysis. a, Gait analysis using the CatWalk XT system. Average speed of tread. dCAM animals 5 wpi, n=4. Data in cm/s: Naïve 40.30±12.78, GFP 24.19±6.25, ALNe-218 25.14±4.74, ALN 26.03±6.69. b, Vertical pole test for dCAM animals. Latency time to turn, n=4. Data in s: Naïve 15.50±8.54, GFP 33.25±22.04, ALNe-218 27.25±14.15, ALN 11.00±7.96. GFP vs ALN P=0.17. c, Phase dispersion left hind paw to right hind paw (LH>RH). Controls: Grey square-naïve, orange triangle-6-OHDA treated animals. Data in %: Naïve 52.42±3.58, 6-OHDA 62.53±4.08. Naïve vs. 6-OHDA P=0.0174. Treatments: Green-GFP, blue-ALNe-218, red-ALN. dCAM: GFP 54.47±3.66, ALNe-218 56.84±7.28, ALN 49.82±5.14. ALN vs. ALNe-218 P=0.0549, multiple comparison ANOVA F(2,20)=3.250. dCAS: GFP 53.77±3.56, ALNe-218 58.04±5.42, ALN 52.38±3.84. ALN vs. ALNe-218 P=0.0653, multiple comparison ANOVA F(2,17)=3.193. Error bars represent mean ±SD.

FIG. 21: The Rosa26 knock-in dCas9 Activator Mouse (dCAM). a, Knock-in of a conditional dCas9-VPR-P2A-SAM expression cassette into the Gt(ROSA)26Sor locus enables flexible multiplexed endogenous gene activation in vitro and in vivo. The cassette is composed of a ubiquitous CAG promoter, a stop cassette (Stop LoxP-puro-stop-LoxP) followed by the FRT flanked SAM activator, a P2A peptide and dCas9-VPR. dCAM x Gfap-Cre mice enable astrocyte-specific dCas9 and activator expression. For in vivo activation, six sgRNAs, driven by different Pol III promoters (H1, hU6, mU6 and 7SK) and the marker gene FLEx-GFP, or split-FLEx-GFP respectively, driven by a CBh promoter are delivered by AAV. b, Multiplexed activation of Ascl1, Lmx1a, NeuroD1 and of Ascl1, Lmx1a, Nr4a2 in primary astrocytic cultures. n=3, representative experiment from 2-3 independent experiments, additional data in supplement. Activation levels are depicted as fold change between cells transfected with and without sgRNAs. All levels were normalized to β-Actin. Error bars represent mean ±SD between technical replicates. c, Experimental Outline: 6-hydroxydopamine (6-OHDA-HCI) is stereotactic injected into the medium forebrain bundle to induce nigrostriatal dopaminergic neurodegeneration. Two weeks later, an AAV expressing gRNAs and a fluorescent reporter is injected into the dorsal striatum. Animals were analyzed after 5 and 13 weeks post injection (wpi) including behavior tests, electrophysiological measurements and immunohistochemistry. Abbreviations: Puro-puromycin resistance, SAM-synergistic activation mediator (MS2: MS2 bacteriophage coat protein, p65: p65 subunit of human NF-KB, HSF1: Heat shock factor 1), P2A-2A self-cleaving peptide, dCas9 - deadCas9 (nuclease-deficient), VPR-VP64: 4× VP16 herpes simplex virus protein vmw65, p65, Rta: Regulator of transcriptional activation, CAG-CMV early enhancer/chicken β actin promoter, CBh-chicken β-actin hybrid promoter. SgRNA expression is driven by the different Pol III promoters (H1, hU6, mU6 and 7SK).

FIG. 22: dCas9 Activator Mouse (dCAM) based reprogramming of astrocytes. a, Representative photomicrographs taken from the dorsal striatum 13 weeks after AAV-injection. In mice injected with GFP control virus, virtually all GFP positive cells depict an astrocytic morphology, many GFP positive cells in ALNe-218 and ALN-treated animals show a neuron-like morphology. b, Immunohistochemical analysis showing GFP⁺/GFAP⁺ double positive cells 13 wpi. Arrows indicate double positive GFP⁺/GFAP⁺ cells, arrowheads indicate GFP⁺/GFAP⁻ cells. Quantification of GFAP+/GFP+cells shows a significant decrease upon ALNe-218 and ALN-treatment (GFP vs. ALNe-218 P=0.0064, GFP vs. ALN P=0.0002 and ALN vs. ALNe-218 P=0.0127). Multiple comparison ANOVA F(2,7)=32.06. c, Double immunostaining for GFP and the neuronal marker NeuN. Arrowheads indicate double positive GFP⁺/NeuN^{+ cell} 13 wpi. Arrow heads indicating GFP⁺/NeuN⁺ cells. Quantification demonstrate a significant increase in NeuN+/GFP+cells upon ALNe-218 and ALN-induction (GFP vs. ALNe-218 P<0.0001, GFP vs. ALN P<0.0001 and ALN vs. ALNe-218 P=0.0012). Multiple comparison ANOVA F(2,6)=170.3. Scale bar indicates 20 μm. Tukey's multiple comparisons test * P<0.05, **P<0.01, ***P<0.001, ****P<0.0001. Error bars represent mean ±SD. d, Double immunostaining for GFP and TH as a marker for dopaminergic neurons in the dorsal striatum of GFP-control, ALNe-218 and ALN-treated animals. Arrowheads indicate GFP⁺/TH⁻ cells with neuronal morphology. No GFP⁺/TH⁺ cell could be detected in any of the experimental groups. Scale bars indicate 20 μm.

FIG. 23: The AAV-split-dCas9 Activator System (AAV-dCAS). a, dCas9 is separated into a N- and a C-terminal part (AAV-N-dCas9^{aa1-673}-N-intein and AAV-C-dCas9^{aa674-1368}-VP64-C-intein), both portions are fused to the corresponding intein-moieties. Upon co-expression intein-mediated trans-splicing leads to reconstitution of Cas9 protein. b, RT-qPCR analysis of Ascl1 induction comparing the activation capacity of full-length versus split-dCas9 in Neuro2A cells (data in fold change normalized to non-activated control; dCas9 6116±847.3, split-dCas9 4415±748.8, n=3). c, Immunocytochemistry analysis of reprogrammed primary astrocytes cells 16 days after lentiviral transduction revealed successful in vitro reprogramming into neurons using CRISPRa. Astrocytes are infected with two lentiviruses expressing dCas9-VPR in a intein-split version similar to the AAV-dCAS system but driven by a Tet-O promoter. Tet-O driven dsRed and Ascl1 cDNA expressing construct were used as negative and positive controls respectively. Arrows indicate single MAP2 positive background neurons, arrowheads indicate double positive induced neuons. d, Schematic representation of the AAV-dCAS system: For induction of up to five endogenous genes plus a GFP reporter, a total of four different AAVs are utilized. dCas9 is delivered by two AAVs, a third AAV is needed for the delivery of the SAM activator. A forth virus contains the reporter gene, while sgRNAs are distributed between the vectors. To ensure cell type specificity upon Cre expression, the N-dCas9 and the GFP are inverted and flanked by two different LoxP sites (LoxX and Lox511). e, Multiplexed activation of Ascl1/Lmx1a/NeuroD1, Ascl1/Lmx1a/Nr4a2 and Ascl1/Lmx1a/ Nr4a2/Pitx3/FoxA2 in primary astrocytic cells. n=2-3 biological replicates, one representative run is shown, additional data in supplement. Activation levels are depicted as fold change between cells transfected with and without sgRNAs. All levels were normalized to β-Actin. Error bars represent mean ±SD between technical replicates. Scale bar indicates 20 μm. Error bars represent mean ±SD. Abbreviations: dN-Cas9-N-terminal dCas9-residues 1-573, N-intein-N-terminal part of DNA polymerase III subunit alpha, dC-Cas9-C-terminal dCas9 residues 574-1368, C-intein-C-terminal part of DNA polymerase III subunit alpha, VP64-4× VP16 herpes simplex virus protein vmw65, p65-p65 subunit of human NF-KB, HSF1-heat shock factor 1, MS2-MS2 bacteriophage coat protein, PAM-protospacer adjacent motif, TSS-transcriptional start site, OE-overexpression, CBh-chicken β-actin hybrid promoter. SgRNA expression is driven by the different Pol III promoters (H1, hU6, mU6 and 7SK).

FIG. 24: AAV-split-dCas9 Activator System (AAV-dCAS) based reprogramming of astrocytes. a, Representative photomicrographs taken from the dorsal striatum 13 weeks after AAV-injection. In the GFP control condition, virtually all GFP positive cells depict an astrocytic morphology, many GFP positive cells in ALNe-218 and ALN-treated animals show a neuron-like morphology. b, Immunohistochemical analysis showing GFP⁺/GFAP⁺ double positive cells 13 wpi. Arrows indicate double positive GFP⁺/GFAP⁺ cells, arrowheads indicate GFP⁺/GFAR cells. Quantification of GFAP⁺/GFP₊ cells shows a significant decrease upon ALNe-218 and ALN-treatment (GFP vs. ALNe-218 P=0.0083, GFP vs. ALN P<0.0001 and ALN vs. ALNe-218 P=0.0006, multiple comparison ANOVA F(2,6)=79.76). c, Photomicrographs showing GFP⁺/NeuN^{+ neurons} 13 wpi. Arrowheads indicating GFP⁺/NeuN⁺ induced neurons. Quantification demonstrate a significant increase in NeuN⁺/GFP⁺ cells upon ALNe-218 and ALN-induction (GFP vs. ALN P=0.0008, ALN vs. ALNe-218 P=0.0092, multiple comparison ANOVA F(2,7)=21.74). d, In ALN-induced neurons, confocal analysis is demonstrating a co-localization of GFP and glutamic acid decarboxylase (Gad65/67), a marker specific for GABAergic neurons. e, Quantification of GAD6567⁺/GFP⁺ cells with neuronal morphology shows that the majority of ALN-induced neurons are colocalizing with this GABAergic marker both in the AAV-dCAS as well as in the dCAM setting. Error bars represent mean ±SD. Tukey's multiple comparisons test * P<0.05, **P<0.01, ***P<0.001, ****P<0.0001.

FIG. 25: Analysis of striatal tissue from ALN reprogrammed dCAM mice by single cell RNA-seq. a, Scheme depicting experimental preparation of cells of 13 wpi mice striatal regions (n=2, one technical replicate). Papain dissociated cells are prepared for scRNA-seq using droplet-based separation and barcoding. Uniform Manifold Approximation and Projection (UMAP) visualization of QC-selected cells for GFP and ALN (n=3,899). Color labeling highlights nine main cell groups based on Leiden clustering and identification based on marker genes. Rectangle highlights astrocytic and neuronal cell clusters. 4,273 highly variable genes (HVG) were detected. b, Subclustering of 1,110 cells identified four groups of astrocytic and neuronal identity. Layout is based on UMAP visualization presented in a. Clustering of markers genes selected based on expression levels between clusters. Expression Z-scores are hierarchically clustered by rows. c, GFP control and ALN reprogrammed cells selected from the neuronal and astrocytic clusters are visualized based on detection of GFP (red cells), marker gene Ascl1, Myt11 and Gad1/Gad2 (Gad1/2) (blue cells), and the co-detection of both (yellow cells). Numbers of GFP positive cells (red), marker gene positive cells (blue) and double positive cells (yellow) are indicated for the astrocytic and neuronal clusters respectively.

FIG. 26: Electrophysiological characterization of AAV-dCAS induced neurons. a, Firing pattern of a neuron reprogrammed by the endogenous activation of Ascl1, Lmx1a, NeuroD1 and expression of miRNA218 (ALNe-218). All analyzed cells (n=10) showed electrophysiological properties of immature neuron/glia-like cells (i.e. lack of APs and a relatively low Rᵢₙ). b, Firing pattern of a neuron reprogrammed by the endogenous activation of Ascl1, Lmx1a, Nr4a2 (ALN). The majority, 14 out of 15 cells, exhibit action potentials, while one showed electrophysiological properties of immature neuron/glia-like cells (i.e. lack of APs and a relatively low Rᵢₙ). Bottom right: example of spontaneous synaptic events recorded from a ALN reprogrammed neuron.

FIG. 27: Rescue of motor behavior in dCAM and AAV-dCAS animals 13 weeks after injection. Gait analysis using the CatWalk XT system reveals motor defects in 6-OHDA lesion model. Both dCAM and AAV-dCAS animals transduce with AAV containing specific gRNAs show a significant improvement in different aspects of voluntary movement like the average speed of tread (a, naive vs. 6-OHDA P=0.0063. AAV-dCAS: GFP vs. ALN P=0.015 multiple comparison ANOVA F(2,17)=12.81) the stride length of front paws (b, naive vs. 6-OHDA P=0.0164. AAV-dCAS: GFP vs. ALN P=0.005, ALN vs. ALNe-218 P=0.0042, multiple comparison ANOVA F(4,22)=9.9) and the duty cycle of left front paws (c, naive vs. 6-OHDA P=0.0096. dCAM: GFP vs. ALN P=0.036 and ALN vs. ALNe-218 P=0.0252, multiple comparison ANOVA F(2,20)=5.199). In contrast to this, dopamine dependent drug-induced behavior does not show rescue effects: d, amphetamine-induced rotation analysis: change in rotational behavior in lesioned animals upon treatment with dopamine releaser substance. Net rotation =ipsilateral rotation-contralateral rotation (naïve vs. 6-OHDA P=0.09). Statistics: naive vs. 6-OHDA unpaired t-test (two-tailed) * P<0.05, ** P<0.01. GFP vs ALN, GFP vs ALNe-218 and ALN vs ALNe-218 Tukey's multiple comparisons test * P<0.05, ** P<0.01. CatWalk error bars represent mean ±SD. Rotation analysis error bars represent mean ±SEM.

FIG. 28: Design and evaluation of the dCAM line. a, The LoxP-flanked puro-stop cassette ensures highly specific knock-in expression. Western blot analysis of targeting construct in Neuro2A cells. Left blot —Test of the P2A sequence for appropriate cleavage. Antibody binds 5″part of the P2A, SAM-5″-P2A and detects a 55 kDa peptide. No uncleaved fusion products are observed. Right blot-Test of the puro-stop-cassette. Without Cre no Cas9 protein is visible b, Variable activation levels can be achieved by removing the FRT-flanked SAM activator via flippase induced recombination. c, Rosa26 knock-in design, homology arms are used 5″arm 1 kb and 3″arm 4 kb long. Southern blot analysis of the founder animals. gDNA digest using EcoRV results in one wild type fragment of 11.5 kb and one 8.7 kb knock-in fragment indicating the heterozygous knock-in in mouse number 3, which was used for further breeding. Genotyping PCR of F1 generation using Cas9 F and Cas9 R primers, 4 (No. 3, 4, 6, 8) out of 10 animals show knock-in. Western blot from primary astrocytes of the dCAM x GFAP-cre line. dCas9 is only detected when Cre was expressed. d, For in vivo activation an AAV containing 6 sgRNAs and a reporter gene can be applied. e, If more than 6 sgRNAs shall be used for in vivo activation two AAVs containing 12 sgRNAs or 6 sgRNAs and a miRNA expression cassette can be applied with a split-reporter gene. AAVs contain sgRNAs, whose expression is driven by the different Pol III promoters (H1, hU6, mU6 and 7SK), and the marker gene FLEx-GFP, respectively split-FLEx-GFP, is expressed by the CBh promoter and also delivered by AAVs. Abbreviations: Puro-puromycin resistance, SAM-synergistic activation mediator (MS2: MS2 bacteriophage coat protein, p65: p65 subunit of human NF-KB, HSF1: Heat shock factor 1), P2A-2A self-cleaving peptide, dCas9-deadCas9 (nuclease-deficient), VPR-VP64: 4× VP16 herpes simplex virus protein vmw65, p65, Rta: Regulator of transcriptional activation, CAG-CMV early enhancer/chicken β actin promoter.

FIG. 29: AAV combinations. A representation of the AAV combinations, which were used for the different approaches and experimental groups with detailed information to promoter and gRNA position and regulatory elements. a, Combinations used for the dCas9 activator mouse experiments. b, Combinations used for the adeno-associated virus (AAV)-based intein-split-dCas9 activator system (AAV-dCAS).

FIG. 30: Evaluation of dCAM x Gfap-Cre primary astrocytes for the activation capacity. a, Multiplexed activation of Ascl1, Lmx1a and Nr4a2; independent replicates: left: Ascl1 6±3, Lmx1a 28±12, Nr4a2 10±3, right: Ascl1 10±7, Lmx1a 22±3, Nr4a2 6±1) b, Multiplexed activation of Ascl1, Lmx1a and NeuroD1. (Ascl1 31±19, Lmx1a 30±23, NeuroD1 206±134). Activation levels are depicted as fold change between cells transfected with and without sgRNAs. All levels were normalized to β-Actin. Error bars represent mean ±SD between technical replicates.

FIG. 31: Evaluation of 6-ODHA induced lesion. a, b, Immunohistochemistry in an animal 14 days after the 6-OHDA injection into the medium forebrain bundle. a, Immunohistochemical staining of dopaminergic lesion using the marker tyrosine hydroxylase (TH). b, Staining with the astrocytic marker GFAP to assess the reactive gliosis. c, Reactive gliosis was assessed via the signal intensity of GFAP stained striata. Naïve, 6 days post lesion (dpl) and 14 dpl animals were analyzed. Per condition data was collected from two animals, from each animal ten images were analyzed, randomly taken in striatal regions. Ipsilateral: Naïve 19.7±1.8, 6 dpl 28.7±6.7, 14 dpl 24.9±6.0, contralateral: Naïve 14.3±4.4, 6 dpl 14.4±2.0, 14 dpl 13.3±1.9.

FIG. 32: Total amount and regional distribution of GFP +cells in vivo in dCAM x Gfap-Cre mice injected with FLEx-GFP reporter. a, GFP +cells in the ipsilateral dorsal striatum of one slide after five weeks of injection. No significant difference could be observed between the different reprogramming conditions and the GFP control. GFP 892.0±85.4, ALNe-218 652.7±193.6, ALN 993.3±106.6. Error bars represent mean ±SD. b, Immunohistochemical staining of GFP positive cells 13 wpi with ALN illustrates the regional distribution of the infected and reprogrammed cells. Quantifications are performed in the dorsal striatum (red dashed line) excluding the subventricular zone. Abbreviations: CX-cortes, CC-corpus callosum.

FIG. 33: In vivo reprogramming 5 weeks after AAV injection in dCAM x Gfap-Cre mice. a, Photomicrographs showing GFP⁺/Gfap⁺ double positive cells 5 wpi. Arrows indicating GFP⁺/Gfap⁻ cells. b, Quantification GFAP⁺/GFP⁺ cells. GFP 97.13±0.45%, ALNe-218 79.33±6.05%, and ALN 86.70±1.90%. GFP vs. ALNe-218 P=0.0025, GFP vs. ALN P=0.03. Multiple comparison ANOVA F(2,6)=17.78. c, Photomicrographs showing

GFP⁺/NeuN^{+ neurons} 5 wpi. Arrow heads indicating GFP⁺/NeuN⁺ cells. d, Quantification NeuN⁺/GFP⁺ cells. GFP 3.9±0.53%, ALNe-218 15.67±0.96% and ALN 14.77±3.09%. GFP vs. ALNe-218 P=0.0007, GFP vs. ALN P=0.001. Multiple comparison ANOVA F(2,6)=35-85. Scale bar indicates 50 μm. Error bars represent mean ±SD. Tukey's multiple comparisons test * P<0.05, **P<0.01, ***P<0.001, ****P<0.0001.

FIG. 34: Activation of endogenous genes using the dCas9-VPR and the SAM activator system in Neuro2A cells. a, Activation of Ascl1. (VPR 82±21, SAM 3158±10, SAM&VPR 10493±432) b, Activation of Ngn2. (VPR 355±17, SAM 2290±476, SAM&VPR 2422±195) c, Activation of MyoD1. (VPR 264±91, SAM 6047±517, SAM&VPR 6285±669) d, Activation of Pou5F1. (VPR 3682±1003, SAM 25041±2507, SAM&VPR 20534±4521). Each gene was activated by two sgRNAs targeting a region -200bp to 1bp upstream of the transcriptional start side. Activation levels are depicted as fold change between cells transfected with and without sgRNAs. All levels were normalized to β-Actin. n=1, with three technical replicates. Error bars represent mean ±SD between technical replicates.

FIG. 35: Evaluation of AAV-dCAS system in vitro. a, Western blot analysis evaluating the FLEx-N-dCas9 system in Neuro2A cells, using a C-Cas9 antibody. b, Western blot analysis evaluating the split-dCas9 system in Neuro2A cells, left blot-N-Cas9 antibody, right blot-C-Cas9 antibody. Correct fusion of the split-dCas9 parts at 175 kDa. c, Immunocytochemistry analysis on primary astrocytic cultures. Activation of Ascl1, Lmx1a and Nr4a2. Upper lane-Transfection of dCas9-activators without sgRNAs. Lower lane -Transfection of dCas9-activators with sgRNAs. Red channel staining for the respective protein. Scale bars indicate 10 μm. d, RT-qPCR levels. Multiplexed activation of Ascl1, Lmx1a, Nr4a2 (Ascl1 103±19, Lmx1a 91±62, Nr4a2 129±16) and Ascl1, Lmx1a, and NeuroD1 (left (1): Ascl1 100±61, Lmx1a 14±7.5, NeuroD1 1542±352, right (2): Ascl1 73±6, Lmx1a 12±3, NeuroD1 1160±142) and Ascl1, Lmx1a, Nr4a2, Pitx3, FoxA2 (Ascl1 167±30, Lmx1a 215±18, Nr4a2 107±10, Pitx3 195±19, FoxA2 32±8) in primary astrocytic cultures. Activation levels are depicted as fold change between cells transfected with and without sgRNAs. All levels were normalized to β-Actin. Error bars represent mean ±SD between technical replicates.

FIG. 36: In vivo reprogramming 5 wpi-AAV-dCAS reprogramming in Gfap-Cre mice. a, Photomicrographs showing GFP⁺/GFAP⁺ cells 5 wpi. Arrows indicating GFP⁺/GFAP⁻ cells. b, Quantification GFAP⁺/GFP₊ cells. GFP 95.37±0.40%, ALNe-218 80.33±1.75% and ALN 84.10±4.16%. GFP vs. ALNe-218 P=0.001, GFP vs. ALN P=0.0045. Multiple comparison ANOVA F(2,6)=26.85. c, Photomicrographs showing GFP⁺/NeuN^{+ neurons} 5 wpi. Arrow heads indicating GFP⁺/NeuN⁺ cells. d, Quantification NeuN⁺/GFP⁺ cells. GFP 4.23±1.55%, ALNe-218 14.10±0.89%, ALN 14.67±1.21%. GFP vs. ALNe-218 P=0.0002, GFP vs. ALN P=0.001. Multiple comparison ANOVA F(2,6)=66.67. Scale bar indicates 50 μm. Error bars represent mean ±SD. Tukey's multiple comparisons test * P<0.05, **P<0.01, ***P<0.001, ****P<0.0001.

FIG. 37: Neurotransmitter identities of reprogrammed neurons using AAV-dCAS. a, b, Confocal images showing co-localization of GFP with specific markers for neurotransmitter subtypes. a, Tyrosine hydroxylase-dopaminergic neurons b, Vesicular glutamate transporter 1-glutamatergic neurons. Scale bars indicate 50 μm.

FIG. 38: Darpp32 staining and quantification 13 wpi. a, b, Evaluation Darpp32 staining in dCAM model. a, Confocal images showing co-localization of GFP and Darpp32. b, Quantification Darpp32⁺/GFP +cells. GFP 4.3±0.5%, ALNe-218 4.76±0.38% and ALN 5.67 ±0.49%. GFP vs. ALN P=0.023. Multiple comparison ANOVA F(2,6)=7.078. c, d, Evaluation Darpp32 staining in AAV-dCAS model. c, Confocal images showing co-localization of GFP and Darpp32. d, Quantification Darpp32⁺/GFP +cells. GFP 3.4±0.1%, ALNe-218 3.97±0.99% and ALN 6.43±0.42%. GFP vs. ALN P=0.0024, ALN vs. ALNe-218 P=0.0067. Multiple comparison ANOVA F(2,6)=20.24. Scale bars indicate 50 μm. Error bars represent mean ±SD. Tukey's multiple comparisons test * P<0.05, **P<0.01, ***P<0.001.

FIG. 39: Phenotypical characterization of AAV-dCAS reprogrammed neurons. Confocal images demonstrating the absence of several interneuron subtype markers in GFP+cells: Parvalbumin (PV), Calretinin (Calb2), Neuropeptide Y (NPY) and Choline acetyl transferase (ChAT). Scale bars indicate 50 μm.

FIG. 40: Quality control of single cell RNA-seq at 13 wpi of AAVs in dCAM x GFAP-Cre mice striatal tissue. a, Number of genes (y-axis) versus count depth (x-axis) per cell. Color highlights fraction of mitochondrial reads. Quality control thresholds of 800 and 250 for number of genes and minimum cell depth are defined, respectively, obtaining 3,899 cells. b, Distributions of count depth for all cells. Inset shows count depth distribution from for all cells with fewer than 4000 counts. The count depth threshold of 800 is shown as a red, vertical line. c, Distribution of number of genes detected per cell. Red line indicates thresholds as in a.

FIG. 41: Rank_genes_groups plots from Scanpy showing the top 25 marker genes using a t-test between log-normalized expression values. a, Top 25 marker genes ranked by their using t-test statistic when comparing normalized cell counts between annotated group against all other groups. b, Same as in a but using four astrocytic-neuronal subclusters (n=1,110 cells). Colors as in FIG. 24 a, b.

FIG. 42: scRNA-seq analysis 13 wpi in dCAM x GFAP-Cre mice. a, Pearson's correlation coefficient of top-10 marker gene expression levels of cells in astrocytic subgroups (n=643) and in neuronal cell subgroups (n=467). b, Counts for GFP +cells (red), markers Cre, Nr4a2, and Lmx1a (blue) and co-detection of cell with both markers (yellow) in GFP control and ALN reprogramming. c, Same as in b, but showing Gad1, Gad2, Th and Slc17a 7 as markers genes.

FIG. 43: Additional electrophysiological measurements 13 and 5 weeks after injection in the AAV-dCAS setting. a, The resting membrane potential (Vₘ in mV) is similar between different reprogramming conditions. b, Input resistance (Rᵢₙ in m0) is significantly different between the different conditions (p=0.002, Kruskall-Wallis test). The input resistance of cells measured in the ALNe-218 condition are similar to immature neurons/glia-like cells, whereas ALN reprogrammed cells exhibit an input resistance within the range of endogenous neurons. Kruskall-Wallis test **P<0.01. Error bars represent mean ±SEM. c, Firing pattern of induced neurons 5 weeks after ALN injection. Neurons exhibit electrophysiological properties of immature neurons (cell 1 exhibited one action potential) respectively of glial cells (cell 2 and 3).

FIG. 44: Motor behavior analysis. a, Gait analysis using the CatWalk XT system. Average speed of tread. dCAM animals 5 wpi, n=4. Data in cm/s: Naïve 40.30±12.78, GFP 24.19±6.25, ALNe-218 25.14±4.74, ALN 26.03±6.69. b, Vertical pole test for dCAM animals. Latency time to turn, n=4. Data in s: Naïve 15.50±8.54, GFP 33.25±22.04, ALNe-218 27.25±14.15, ALN 11.00±7.96. GFP vs ALN P=0.17. c, Phase dispersion left hind paw to right hind paw (LH>RH). Controls: Grey square-naïve, orange triangle-6-OHDA treated animals. Data in %: Naïve 52.42±3.58, 6-OHDA 62.53±4.08. Naïve vs. 6-OHDA P=0.0174. Treatments: Green-GFP, blue-ALNe-218, red-ALN. dCAM: GFP 54.47±3.66, ALNe-218 56.84±7.28, ALN 49.82±5.14. ALN vs. ALNe-218 P=0.0549, multiple comparison ANOVA F(2,20)=3.250. dCAS: GFP 53.77±3.56, ALNe-218 58.04±5.42, ALN 52.38±3.84. ALN vs. ALNe-218 P=0.0653, multiple comparison ANOVA F(2,17)=3.193. Error bars represent mean ±SD.

### DETAILED DESCRIPTION OF THE INVENTION

### Definitions

As referred herein “EC numbers” (Enzyme Commission numbers) may be used to refer to enzymatic activity according to the Enzyme nomenclature database, Release of Feb. 26, 2020 (e.g., available at https://enzyme.expasy.org/). The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively.

The term “EC: 3.1.-.-.” as used herein can be interchangeably used with the term “”EC: 3.1.X.Y., wherein X is independently selected from 1 to 31 and Y is independently selected from 1 to 114″. The term “EC: 3.1.-.-.” may refer to endonuclease activity of Cas9.

The term “AAV” may refer to Adeno-associated virus.

As used herein, the terms “nucleic acids” or “nucleotide sequences” may refer to DNA molecules (e.g. cDNA or genomic DNA), RNA (mRNA), combinations thereof or hybrid molecules comprised of DNA and RNA. The nucleic acids can be double- or single-stranded and may contain double- and single-stranded fragments at the same time. Most preferred are double stranded DNA molecules.

The term “endonuclease activity” may refer to enzymatic activity that cleave the phosphodiester bond within a polynucleotide chain.

The terms “intein” or “intein activity” may refer to polypeptides (e.g., co-called protein introns) capable of excising themselves out of a polypeptide sequence and joining the remaining flanking regions (e.g., exteins) with a peptide bond.

The term “intein activity” may refer to protein trans-splicing activity.

The term “split-intein” may refer to a sub-group of inteins that are present in two separate complementary entities and catalyze protein splicing in trans upon association of said two complementary entities.

The terms “guide RNA” or “gRNA” may refer to non-coding short RNA sequences which bind to the complementary target DNA sequences and confer target sequence specificity to the CRISPR-Cas9 system.

The term “Cas9” may refer to CRISPR associated protein 9. Cas9 is a dual RNA-guided DNA endonuclease enzyme associated with the Clustered Regularly Interspaced Short Palindromic Repeats (CRISPR).

The term “trans-activating activity” may refer to transcription activation (e.g., increasing a rate of gene expression), e.g., in trans.

The term “aptamer” may refer to a short segment of DNA (e.g., oligonucleotide), RNA or peptide that binds to a specific molecular target (such as a protein).

The terms “transcription co-activator” or “coactivator” may refer a type of transcriptional co-regulator that binds to an activator (a transcription factor) to increase the rate of transcription of a gene or set of genes.

The terms “synergistic activation mediator” or “SAM” may refer to potent transcriptional activation protein complex.

The term “polypeptide” is equally used herein with the term “protein”. Proteins (including fragments thereof, preferably biologically active fragments, and peptides, usually having less than 30 amino acids) comprise one or more amino acids coupled to each other via a covalent peptide bond (resulting in a chain of amino acids). The term “polypeptide(s)” as used herein describes a group of molecules, which, for example, consist of more than 30 amino acids. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo-or hetero-trimers etc. An example for a hetero-multimer is an antibody molecule, which, in its naturally occurring form, consists of two identical light polypeptide chains and two identical heavy polypeptide chains. The terms “polypeptide” and “protein” also refer to naturally modified polypeptides/proteins wherein the modification is affected e.g. by post-translational modifications like glycosylation, acetylation, phosphorylation and the like. Such modifications are well known in the art.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used may be gap open penalty of 10, gap extension penalty of 0.5, and the EBLOSUM62 (EMBOSS version of BLOSUM62) substitution matrix. The output of Needle labeled “longest identity” (obtained using the no-brief option) is used as the percent identity and is calculated as follows:

(Identical Residues×100)/(Length of Alignment−Total Number of Gaps in Alignment).

Alternatively, the parameters used may be gap open penalty of 10, gap extension penalty of 0.5, and the EDNAFULL (EMBOSS version of NCBI NUC4.4) substitution matrix. The output of Needle labeled “longest identity” (obtained using the no-brief option) is used as the percent identity and is calculated as follows:

(Identical Deoxyribonucleotides×100)/(Length of Alignment−Total Number of Gaps in Alignment).

Expression: The term “expression” includes any step involved in the production of a variant (polypeptide) including, but not limited to, transcription, post-transcriptional modification, translation, post-translational modification, and secretion.

Expression vector: The term “expression vector” may refer to a linear or circular DNA molecule that comprises a polynucleotide encoding a variant (polypeptide) and is operably linked to control sequences that provide for its expression, in particular for its transcription.

Fragment: The term “fragment” may refer to a polypeptide having one or more (e.g. several) amino acids absent from the amino and/or carboxyl terminus of a mature polypeptide; wherein the fragment has an activity as described elsewhere herein.

Host cell: The term “host cell” may refer to any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication, e.g., recombinant or transgenic host cell.

Nucleic acid construct: The term “nucleic acid construct” may refer to a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” may refer to a configuration in which a control sequence is placed at an appropriate position relative to the coding sequence of a polynucleotide such that the control sequence directs expression of the coding sequence.

Control sequences: The term “control sequences” as used herein may refer to nucleic acid sequences necessary for expression of a polynucleotide encoding a variant (polynucleotide) of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, pro-peptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide of the present invention.

Parkinson's disease and the associated disturbance in movement coordination and behavior are provoked by the loss of dopaminergic neurons in the SNpc. To date, the prevailing paradigm of disease treatment is the symptomatic management by direct interference of the dopaminergic system to restore dopamine levels in the affected striata through drug treatment or transplantation of dopaminergic neurons. In the course of the present invention genetic tools to reprogram striatal astrocytes into mature neurons by the aCRISPR-mediated activation of multiple endogenous transcription factors, such as Ascl1, Lmx1a and Nr4a2 (ALN), or Ascl1, Lmx1a, NeuroD1 together with miRNA218 (ALNe-218) were developed. The conventional reprogramming approaches use the ectopic expression of the gene coding sequences (cDNA), making multiplexing of several genes difficult if not impossible, especially when large genes have to be expressed. In contrast, the aCRISPR platform allows multiplexed activation of many endogenous genes solely by introducing sgRNAs, with a fixed cargo size for each gene, such that the endogenous transcriptional machinery can be co-opted to execute complex genetic splicing patterns. Herein, it has been shown that two distinct approaches based on CRISPR-mediated gene activation are suitable to achieve successful treatment of a murine toxin-induced PD model. For the dCAM mouse line a Rosa26 knock-in strategy of a Cre- and Flpe-dependent dual activator system was followed, harbouring the VPR and SAM activator complexes where the defined integration and the optional two-fold mode of activation are the prominent features differentiating the transgenic line developed in the course of the present invention from the recently reported SPH transgenic mouse line. After confirming the technical and biological functionality of the dCAM approach, the toolbox was expanded by developing an AAV-based split-dCas9/SAM system, making it versatile and applicable across species with minimal modifications. Strikingly, with the split-dCas9 AAV-based system (AAV-dCAS) it was possible to recapitulate the results obtained with dCAM, confirming the functionality of the aCRISPR approach to reprogram in vivo striatal astrocytes into induced neurons. At 13 wpi the combination ALN was capable to generate functional neurons with mature electrophysiological properties, whereas cells reprogrammed by ALNe-218 exhibited characteristics reminiscent of astrocytes or immature neurons. Furthermore, only ALN induced neurons led to an improvement in voluntary motor behavior, and a balancing of the axial symmetry-. This behavioral rescue could be observed to a similar extent, both in dCAM as well as AAV-dCAS animals confirming the functionality of the aCRISPR-mediated activation approach. The de novo induced neurons were not immunoreactive for the dopaminergic marker TH. Nevertheless, independent of reprogramming, we observe TH+ neurons in the striatum, which may either emerge due to the 6-OHDA toxin treatment or represent naturally occurring TH+ interneurons within the striatum. Although this is contradictory the previously reported formation of dopaminergic neurons based on ALNe-218 overexpression mediated reprogramming, this discrepancy may be explained by the different reprogramming system used and presumably diverse reprogramming kinetics. Furthermore, the FLEx-GFP marker employed in this study, allows the definite identification of induced neurons and its demarcation from reprogramming independent TH+ neurons. Interestingly, scRNA-seq analysis, as well as immunological staining, revealed a GABAergic identity of the reprogrammed neurons. This is indicating that the regional identity of the targeted astrocytes is a predominant factor for the determination of the final neuronal subtype. The induced neurons were not positive for DARPP32, a marker for striatal medium spiny neurons representing the main neuronal class within the striatum, nor did they exhibit standard electrophysiological properties of this particular neuronal subtype. This indicates that the reprogrammed neurons presumably differentiate into a distinct GABAergic interneuron population, capable of modulating striatal circuits. Furthermore, these electrophysiological properties are distinct from PV+ interneurons, which have been shown by a recent publication to arise during ALN overexpression in NG2+ oligodendrocyte precursors, which may be explained by the different starter cell populations. The major originality of this study lies in the fact, that the aCRISPR induced ALN combination in the striatum, using either dCAM or AAV-dCAS, induces specific GABAergic neurons, capable of alleviating motor behaviour symptoms in a 6-OHDA model. This is surprising, since the research focus so far has been on the restoration of the dopaminergic drive to alleviate motor symptoms. However, it has been reported that dopamine depletion in 6-OHDA toxin treated PD rodent models has a strong effect on striatal circuits. Specifically, increased excitatory cholinergic and reduced inhibitory GABAergic signals have been observed. In addition, most of the basal striatal excitatory drive arising from cholinergic interneurons is balanced by a concomitant GABAergic inhibition; this signalling is impaired by dopamine deprivation38/Furthermore, integrity of the fast spiking striatal GABAergic interneurons has been shown to depend on dopaminergic input from SNpc39. Altogether, these reports as well as our own findings suggest, that the imbalance in striatal micro circuitry—including impaired GABAergic signalling—contribute to the altered motor behaviour in parkinsonian state. Therefore, restoration or reinforcing of GABAergic inhibition in the striatum is attractive as a novel therapeutic concept for PD.

In summary, herein for the first time it was shown that a rescue of PD motor behaviour deficits can be achieved by the direct conversion of endogenous astrocytes into functional GABAergic neurons via an aCRISPR mediated induction of the reprogramming factors Ascl1, Lmx1a and Nr4a2.

### Embodiments of the Invention

In some aspects/embodiments, the present invention relates to a plurality of separate adeno-associated viruses (AAVs) comprising: (i) a first AAV comprising a first nucleic acid encoding a first portion of a Cas9 protein devoid of endonuclease activity; (ii) a second AAV comprising a second nucleic acid encoding a second portion of a Cas9 protein devoid of endonuclease activity; (iii) a third AAV comprising a third nucleic acid encoding a synergistic activation mediator (SAM) complex and a single guide RNA (sgRNA) comprising at least two aptamers, each capable of binding two MS2 coactivator proteins, wherein the first portion of said Cas9 protein devoid of endonuclease activity and the second portion of said Cas9 protein devoid of endonuclease activity, when joined together, form a Cas9 protein devoid of endonuclease activity.

In some aspects/embodiments, the first portion of the Cas9 protein of the present invention is the N-terminal lobe of the Cas9 protein and the second portion of the Cas9 protein of the present invention is the C-terminal lobe of the Cas9 protein.

In some aspects/embodiments, the first portion of the Cas9 protein is the N-terminal lobe of the Cas9 protein up to amino acid 573 and the second portion of the Cas9 protein is the C-terminal lobe of the Cas9 protein beginning at amino acid 574.

In some aspects/embodiments, the first nucleic acid encodes a first portion of the Cas9 protein having a first split-intein and wherein the second nucleic acid encodes a second portion of the Cas9 protein having a second split-intein complementary to the first split-intein, wherein the first portion of the Cas9 protein and the second portion of the Cas9 protein, when joined together, form the Cas9 protein.

In some aspects/embodiments, the split-intein polypeptides of the present invention are selected from the group consisting of: Nostoc punctiforme (Npu) strain PCC73102 split-inteins, gp41-1 inteins, NrdJ-1 inteins, IMPDH-1 inteins, HwarPolA29,62 inteins.

In some aspects/embodiments, the the first nucleic acid encodes a first portion of the Cas9 protein having a Rhodothermus marinus N-split-intein Rma IntN and wherein the second nucleic acid encodes a second portion of the Cas9 protein having a Rhodothermus marinus C-split-intein Rma IntC, wherein the first portion of the Cas9 protein and the second portion of the Cas9 protein, when joined together, form the Cas9 protein.

In some aspects/embodiments, the SAM complex of the present invention comprises a MS2 coat protein fused to the p65 subunit of NF-kappaB and the activation domain of human heat-shock factor 1 (HSF1).

In some aspects/embodiments, the second portion of a Cas9 protein devoid of endonuclease activity is fused to a transcription activation domain.

In some aspects/embodiments, the third nucleic acid further encodes a transcription activation domain.

In some aspects/embodiments, the transcription activation domain is a quadruple VP16 (VP64) domain.

In some aspects/embodiments, the Cas9 is a Type II CRISPR system Cas9.

In some aspects/embodiments, the invention relates to a plurality of separate adeno-associated viruses (AAVs, e.g., AAV2 and/or AAVS serotypes) comprising: (i) a first AAV (e.g., AAV2 or AAVS) comprising a first nucleic acid encoding a first portion of a Cas9 protein devoid of endonuclease activity; optionally, said first nucleic acid further encoding a first split-intein polypeptide (e.g., an N-intein polypeptide), preferably said first split-intein polypeptide having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with the polypeptide having SEQ ID NO: 10 (N-Split-Intein) and having the intein activity (e.g., protein trans-splicing activity); optionally, said first nucleic acid further encoding one or more guide RNAs (gRNAs), preferably said first nucleic acid is up to about 4.5 Kb in size; further preferably said first portion of said Cas9 protein devoid of said endonuclease activity is devoid of an enzymatic activity having EC: 3.1.-.-.; most preferably said first nucleic acid encoding the polypeptide having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with the polypeptide having SEQ ID NO: 2 (N-dCas9-N-intein); (ii) a second AAV (e.g., AAV2 or AAVS) comprising a second nucleic acid encoding a second portion of a Cas9 protein devoid of endonuclease activity; optionally, said second nucleic acid further encoding a second split-intein polypeptide having complementarity to said first split-intein (e.g., a C-intein), preferably said second split-intein polypeptide having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with the polypeptide having SEQ ID NO: 12 (C-Split-lntein) and having the intein activity (e.g., protein trans-splicing activity); optionally, said second nucleic acid further encoding at least one polypeptide having a trans-activating activity (e.g., activating transcription) and/or one or more guide RNAs (gRNAs), preferably said second nucleic acid is up to about 4.5 Kb in size; further preferably said at least one polypeptide having said trans-activating activity having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with the polypeptide having SEQ ID NO: 20 (VP16); further preferably said second portion of said Cas9 protein devoid of said endonuclease activity is devoid of an enzymatic activity having EC: 3.1.-.-.; most preferably said second nucleic acid encoding the polypeptide having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with the polypeptide having SEQ ID NO: 4 (C-dCas9-C-intein-VP64); (iii) a third AAV comprising a third nucleic acid encoding at least one polypeptide having a trans-activating activity (e.g., activating transcription) and capable of binding to and/or associating with an at least one guide RNA (gRNA) and/or said first and/or second portion of said Cas9 protein, wherein said third nucleic acid further encoding at least one guide RNA (gRNA) comprising at least one aptamer capable of binding at least one transcription co-activator protein (e.g., said co-activator protein comprising one or more of the following: (i) a polypeptide having at least 80% (e.g., at least 85%, 90%, 95% or 100%) sequence identity to the MS2 adaptor polypeptide having SEQ ID NO: 14; (ii) a polypeptide having at least 80% (e.g., at least 85%, 90%, 95% or 100%) sequence identity to MS2-p65 polypeptide having SEQ ID NO: 16; and/or (iii) a polypeptide having at least 80% (e.g., at least 85%, 90%, 95% or 100%) sequence identity to MS2-p65-HSF1 polypeptide having SEQ ID NO: 18; (iv) a fusion of a suitable adaptor polypeptide (e.g., least 80% sequence identity to SEQ ID NO: 14) with a suitable activator polypeptide, e.g., p65 and/or HSF1), preferably said third nucleic acid encoding a synergistic activation mediator (SAM) complex and at least one guide RNA (gRNA) comprising at least one aptamer capable of binding at least one co-activator protein (e.g., said co-activator protein comprising one or more of the following: (i) a polypeptide having at least 80% (e.g., at least 85%, 90%, 95% or 100%) sequence identity to the MS2 adaptor polypeptide having SEQ ID NO: 14; (ii) a polypeptide having at least 80% (e.g., at least 85%, 90%, 95% or 100%) sequence identity to MS2-p65 polypeptide having SEQ ID NO: 16; and/or (iii) a polypeptide having at least 80% (e.g., at least 85%, 90%, 95% or 100%) sequence identity to MS2-p65-HSF1 polypeptide having SEQ ID NO: 18; (iv) a fusion of a suitable adaptor polypeptide (e.g., least 80% sequence identity to SEQ ID NO: 14) with a suitable activator polypeptide, e.g., p65 and/or HSF1), most preferably comprising at least two aptamers, each capable of binding at least two co-activator proteins (e.g., said co-activator protein comprising one or more of the following: (i) a polypeptide having at least 80% (e.g., at least 85%, 90%, 95% or 100%) sequence identity to the MS2 adaptor polypeptide having SEQ ID NO: 14; (ii) a polypeptide having at least 80% (e.g., at least 85%, 90%, 95% or 100%) sequence identity to MS2-p65 polypeptide having SEQ ID NO: 16; and/or (iii) a polypeptide having at least 80% (e.g., at least 85%, 90%, 95% or 100%) sequence identity to MS2-p65-HSF1 polypeptide having SEQ ID NO: 18; (iv) a fusion of a suitable adaptor polypeptide (e.g., least 80% sequence identity to SEQ ID NO: 14) with a suitable activator polypeptide, e.g., p65 and/or HSF1), (iv) optionally, a fourth AAV comprising a fourth nucleic acid encoding a reporter polypeptide, preferably said reporter polypeptide is a fluorescent protein, further preferably said fluorescent protein is a green fluorescence protein; wherein said first portion of said Cas9 protein devoid of endonuclease activity and said second portion of said Cas9 protein devoid of endonuclease activity, when joined together, form a Cas9 protein devoid of endonuclease activity, preferably said formed Cas9 protein is capable of binding DNA, further preferably said formed Cas9 protein having at least 80% identity to SEQ ID NO: 27 (e.g., Cas9 having UniProtKB Accession Number Q99ZW2).

In some aspects/embodiments, the first portion of the Cas9 protein is the N-terminal lobe of the Cas9 protein up to amino acid 573 and the second portion of the Cas9 protein is the C-terminal lobe of the Cas9 protein beginning at amino acid 574; or the first portion of the Cas9 protein is the N-terminal lobe of the Cas9 protein up to amino acid 637 and the second portion of the Cas9 protein is the C-terminal lobe of the Cas9 protein beginning at amino acid 638.

In some aspects/embodiments, the first nucleic acid encodes a first portion of the Cas9 protein having a first split-intein and wherein the second nucleic acid encodes a second portion of the Cas9 protein having a second split-intein complementary to the first split-intein, wherein the first portion of the Cas9 protein and the second portion of the Cas9 protein, when joined together, form the Cas9 protein (e.g., by intein-mediated trans-splicing).

In some aspects/embodiments, the first nucleic acid encodes a first portion of the Cas9 protein having a Nostoc punctiforme (Npu) strain PCC73102 N-split-intein IntN and wherein the second nucleic acid encodes a second portion of the Cas9 protein having a Nostoc punctiforme (Npu) strain PCC73102 C-split-intein IntC, wherein the first portion of the Cas9 protein and the second portion of the Cas9 protein, when joined together, form the Cas9 protein.

In some aspects/embodiments, wherein the SAM complex comprises a MS2 coat protein (e.g., having SEQ ID NO: 14) fused to the p65 subunit of NF-kappaB (e.g., forming SEQ ID NO: 16) and the activation domain of human heat-shock factor 1 (HSF1) (e.g., forming SEQ ID NO: 18).

In some aspects/embodiments, second portion of a Cas9 protein devoid of endonuclease activity is fused to a transcription activation domain, preferably said transcription activation domain having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with the polypeptide having SEQ ID NO: 20 (VP16).

In some aspects/embodiments, the present invention relates to composition, kit, expression system or recombinant host cell (e.g., isolated recombinant host cell) comprising the plurality of AAV of the present invention, preferably said composition, kit, expression system or recombinant host cell is pharmaceutical and/or diagnostic composition, kit, expression system or recombinant host cell, further preferably said composition, kit, expression system or recombinant host cell further comprising a reporter, further preferably said reporter is a fluorescent protein, further preferably said fluorescent protein is a green fluorescence protein.

In some aspects/embodiments, the plurality of AAVs, composition, kit, expression system or recombinant host cell of the present invention for use as a medicament (e.g., in vivo) and/or in therapy (e.g., in vivo).

In some aspects/embodiments, the present invention relates to a method for reprogramming and/or modifying a cell, said method comprising: (a) providing: (i) a cell; and (ii) the plurality of AAVs, composition, kit, expression system or recombinant host cell of the present invention; (b) applying and/or expressing (ii) to/in (i); preferably said cell is an astrocyte, further preferably said cell is reprogrammed into a neuron, most preferably said astrocyte is reprogrammed into a neuron.

In some aspects/embodiments, the plurality of AAVs of the present invention, composition, kit, expression system and/or recombinant host cell of the present invention is, for use in one or more of the following methods: (i) method of treatment, amelioration, prophylaxis and/or diagnostics of a neurodegenerative disease, cancer, cardiovascular disease, metabolic disease, monogenic disorder (e.g., single-gene associated disorder, e.g., Osteogenesis Imperfecta (OGI), Retinoblastoma (RB), Cystic Fibrosis, Thalassemia, Fragile X Syndrome (FXS), Hypophosphatemia, Hemophilia and Ichthyosis) and/or diabetes, preferably said neurodegenerative disease is selected from the group consisting of: Parkinson's disease, Parkinsonism, Parkinson-plus syndrome, Alzheimer's disease, Amyotrophic lateral sclerosis (ALS) and Huntington's disease; (ii) method for re-programming and/or modifying a cell, preferably an astrocyte, further preferably into a neuron; (iii) method for inducing and/or modifying expression of one or more genes of interest (e.g., endogenous, e.g., transcription factors, e.g., one or more of the following: Achaete-scute homolog 1 (Ascl1, e.g., UniProtKB-P50553 or Q02067), LIM homeobox transcription factor 1-alpha (Lmx1a, e.g., UniProtKB-Q8TE12 or Q9JKU8), Nurr1 (e.g., UniProtKB-P43354), preferably or alternatively Achaete-scute homolog 1 (Ascl1, e.g., UniProtKB-P50553 or Q02067), LIM homeobox transcription factor 1-alpha (Lmx1a, e.g., UniProtKB-Q8TE12 or Q9JKU8), Neurogenic differentiation factor 1 (Neurod1, e.g., UniProtKB-Q13562 or Q60867), miRNA218 (e.g., NR_029632, NR_029799.1); (iv) method for cell-replacement and/or transplantation; (v) method for somatic reprogramming of a cell; preferably an astrocyte, further preferably into a neuron; (vi) method for genome and /or transcriptome modification, and/or gene therapy; (vii) method of screening (e.g., guide RNAs) and/or monitoring gene expression; preferably said composition or kit further comprising a reporter, further preferably said reporter is a fluorescent protein, further preferably said fluorescent protein is a green fluorescence protein; (viii) method for producing a neuron; (viii) in a method according to any one of preceding items; (ix) said method is an in vitro, in vivo or ex vivo method; (x) in any combination of (i)-(x).

In some aspects/embodiments, the present invention relates to use of the plurality, composition, kit, expression system or recombinant host cell of the present invention, for one or more of the following: (i) for reprogramming and/or modifying a cell, preferably an astrocyte, further preferably into a neuron; (ii) for inducing and/or modifying expression of one or more genes of interest (e.g., endogenous, e.g., transcription factors, e.g., one or more of the following: Achaete-scute homolog 1 (Ascl1 , e.g., UniProtKB-P50553 or Q02067), LIM homeobox transcription factor 1-alpha (Lmx1a, e.g., UniProtKB-Q8TE12 or Q9JKU8), Nurr1 (e.g., UniProtKB-P43354), preferably or alternatively Achaete-scute homolog 1 (Ascl1 , e.g., UniProtKB-P50553 or Q02067), LIM homeobox transcription factor 1-alpha (Lmx1a, e.g., UniProtKB-Q8TE12 or Q9JKU8), Neurogenic differentiation factor 1 (Neurod1, e.g., UniProtKB-Q13562 or Q60867), a ncRNA (non-coding RNA, e.g., miRNA), miRNA218 (e.g., NR 029632, NR_029799.1); (iii) for cell-replacement and/or transplantation; (iv) for somatic reprogramming of a cell; preferably said cell is selected from the group consisting of: an astrocyte, cardiomyocyte, adipocyte, muscle cell, osteoclast, osteoblast, osteocytes, a blood cell (e.g., white blood cell), a skin cell, a stem cell, further preferably said astrocyte is reprogrammed into a neuron; (v) for genome modification and/or gene therapy; (vi) for producing a neuron; (vii) as a medicament and/or in therapy; (viii) for treatment and amelioration, prophylaxis and/or diagnostics of a degenerative disease (e.g., neurodegenerative disease), cancer, cardiovascular disease, metabolic disease, monogenic disorder (e.g., single-gene associated disorder, e.g., Osteogenesis Imperfecta (OGI), Retinoblastoma (RB), Cystic Fibrosis, Thalassemia, Fragile X Syndrome (FXS), Hypophosphatemia, Hemophilia and Ichthyosis) and/or diabetes, preferably said neurodegenerative disease is selected from the group consisting of: Parkinson's disease, Parkinsonism, Parkinson-plus syndrome, Alzheimer's disease, Amyotrophic lateral sclerosis (ALS) and Huntington's disease; (ix) in a method of the present invention; (x) for in vitro, in vivo or ex vivo use; (xi) in any combination of (i)-(x).

In some aspects/embodiments, the present invention relates to Split-dCas9 and SAM packaged in a plurality of AAV viruses that can be used, for reprogramming cells and/or for in vivo-cell therapy.

In some aspects/embodiments, the nucleic acids of the present invention (e.g., first, second, third and/or fourth) are operably linked to a control sequence, preferably operably linked to any suitable promoter (e.g., CBh-chicken β-actin hybrid promoter or human glial fibrillary acidic protein (GFAP) promoter).

In some aspects/embodiments, the plurality, composition, kit, expression system or recombinant host cell of the present invention are particularly suitable for use as an in vivo medicament and/or in vivo therapy.

In some aspects/embodiments, the AAVs of the present invention are selected from the group consisting of: AAV1, AAV2, AAV4, AAV5, AAV7, AAV8, AAV9, preferably AAV2 and AAV5.

In some aspects/embodiments, the invention relates to SEQ ID NOs: 1-27 which are embodiments of the present invention.

In some aspects/embodiments, the nucleic acid of the invention (e.g., first, second, third and/or fourth comprises, consists of or encodes: one or more of the sequences having SEQ ID NO: 1-27.

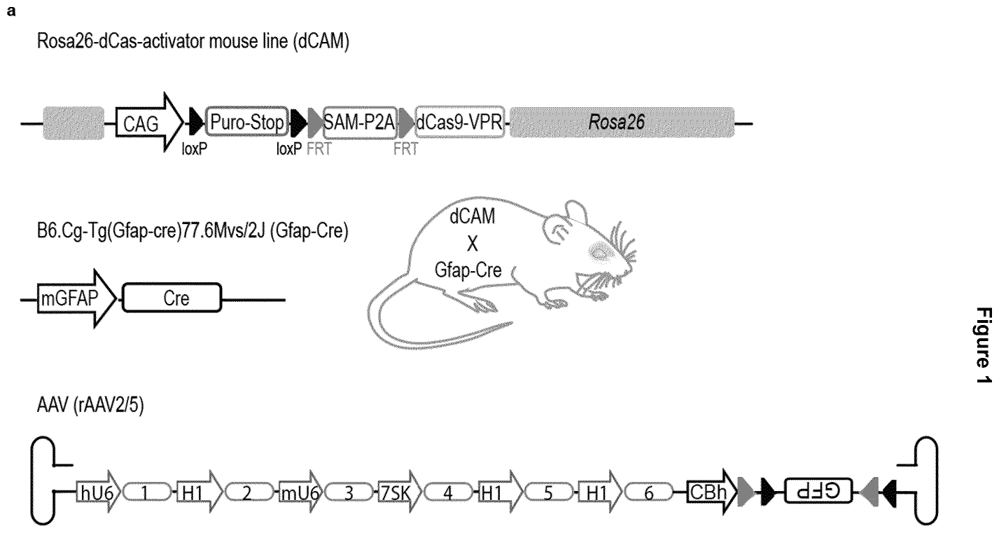

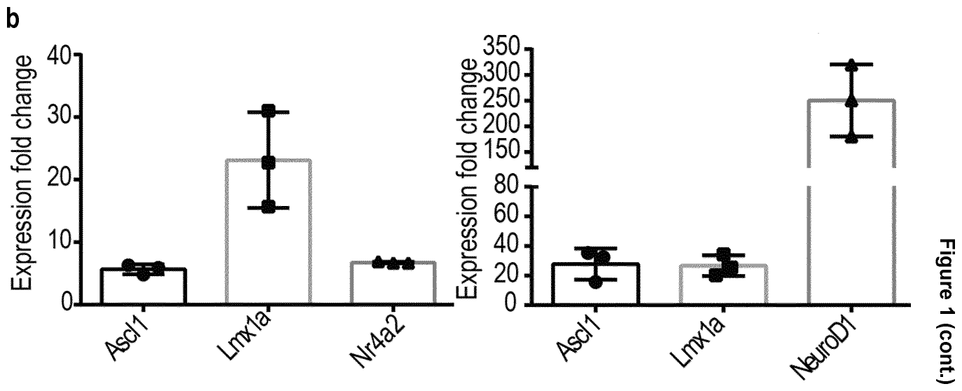

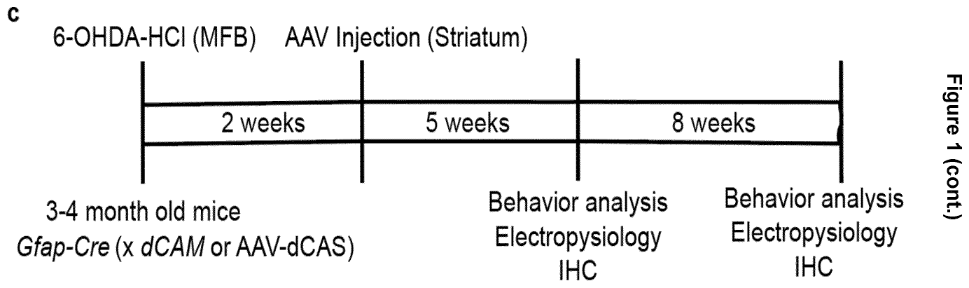

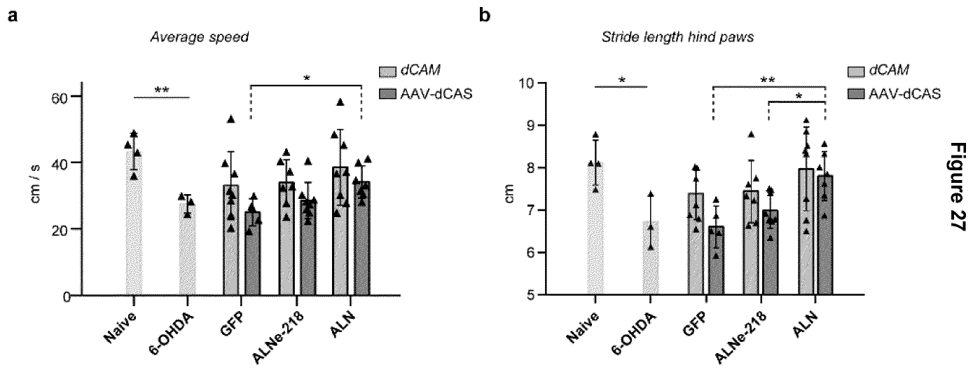

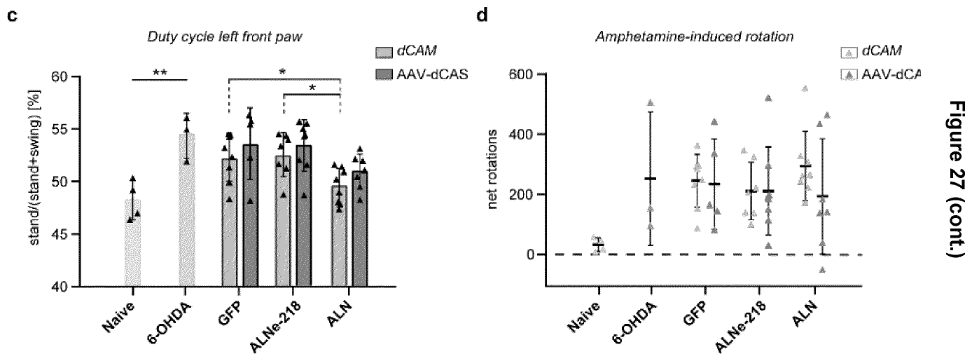

In some aspects/embodiments, the invention relates to FIGS. 1-44, which depict embodiments of the present invention.

In some aspects/embodiments, the nucleic acids of the present invention (e.g., first, second, third and/or fourth) encode one or more gRNAs.

In some aspects/embodiments, first nucleic acid further encoding a first split-intein polypeptide (e.g., an N-intein polypeptide), preferably said first split-intein polypeptide having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with the polypeptide having SEQ ID NO: 10 (N-Split-Intein) and having the intein activity (e.g., protein trans-splicing activity).

In some aspects/embodiments, the first nucleic acid further encoding one or more guide RNAs (gRNAs), preferably said first nucleic acid is up to about 4.5 Kb in size.

In some aspects/embodiments, the first portion of said Cas9 protein devoid of said endonuclease activity is devoid of an enzymatic activity having EC: 3.1.-.-..

In some aspects/embodiments, the first nucleic acid encoding the polypeptide having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with the polypeptide having SEQ ID NO: 2 (N-dCas9-N-intein).

In some aspects/embodiments, the second nucleic acid further encoding a second split-intein polypeptide having complementarity to said first split-intein (e.g., a C-intein), preferably said second split-intein polypeptide having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with the polypeptide having SEQ ID NO: 12 (C-Split-Intein) and having the intein activity (e.g., protein trans-splicing activity).

In some aspects/embodiments, the second nucleic acid further encoding at least one polypeptide having a trans-activating activity (e.g., activating transcription) and/or one or more guide RNAs (gRNAs), preferably said second nucleic acid is up to about 4.5 Kb in size; further preferably said at least one polypeptide having said trans-activating activity having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with the polypeptide having SEQ ID NO: 20 (VP16);

In some aspects/embodiments, the second portion of said Cas9 protein devoid of said endonuclease activity is devoid of an enzymatic activity having EC: 3.1.-.-..

In some aspects/embodiments, the second nucleic acid encoding the polypeptide having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with the polypeptide having SEQ ID NO: 4 (C-dCas9-C-intein-VP64).

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The invention is also characterized by the following items:

1. A plurality of separate adeno-associated viruses (AAVs, e.g., AAV2 and/or AAVS serotypes) comprising:
(i) a first AAV (e.g., AAV2 or AAVS) comprising a first nucleic acid encoding a first portion of a Cas9 protein devoid of endonuclease activity;
   optionally, said first nucleic acid further encoding a first split-intein polypeptide (e.g., an N-intein polypeptide), preferably said first split-intein polypeptide having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) sequence identity with the polypeptide having SEQ ID NO: 10 (N-Split-lntein) and having the intein activity (e.g., protein trans-splicing activity);
   optionally, said first nucleic acid further encoding one or more guide RNAs (gRNAs), preferably said first nucleic acid is up to about 4.5 Kb in size;
   further preferably said first portion of said Cas9 protein devoid of said endonuclease activity is devoid of an enzymatic activity having EC: 3.1.-.-.;
   most preferably said first nucleic acid encoding the polypeptide having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) sequence identity with the polypeptide having SEQ ID NO: 2 (N-dCas9-N-intein);
(ii) a second AAV (e.g., AAV2 or AAVS) comprising a second nucleic acid encoding a second portion of a Cas9 protein devoid of endonuclease activity;
   optionally, said second nucleic acid further encoding a second split-intein polypeptide having complementarity to said first split-intein (e.g., a C-intein), preferably said second split-intein polypeptide having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) sequence identity with the polypeptide having SEQ ID NO: 12 (C-Split-lntein) and having the intein activity (e.g., protein trans-splicing activity);
   optionally, said second nucleic acid further encoding at least one polypeptide having a trans-activating activity (e.g., activating transcription) and/or one or more guide RNAs (gRNAs), preferably said second nucleic acid is up to about 4.5 Kb in size; further preferably said at least one polypeptide having said trans-activating activity having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) sequence identity with the polypeptide having SEQ ID NO: 20 (VP16);
   further preferably said second portion of said Cas9 protein devoid of said endonuclease activity is devoid of an enzymatic activity having EC: 3.1.-.-.;
   most preferably said second nucleic acid encoding the polypeptide having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) sequence identity with the polypeptide having SEQ ID NO: 4 (C-dCas9-C-intein-VP64);
(iii) a third AAV comprising a third nucleic acid encoding at least one polypeptide having a trans-activating activity (e.g., activating transcription) and capable of binding to and/or associating with an at least one guide RNA (gRNA) and/or said first and/or second portion of said Cas9 protein, wherein said third nucleic acid further encoding at least one guide RNA (gRNA) comprising at least one aptamer capable of binding at least one transcription co-activator protein (e.g., said co-activator protein comprising one or more of the following: (i) a polypeptide having at least 80% (e.g., at least 85%, 90%, 95% or 100%) sequence identity to the MS2 adaptor polypeptide having SEQ ID NO: 14; (ii) a polypeptide having at least 80% (e.g., at least 85%, 90%, 95% or 100%) sequence identity to MS2-p65 polypeptide having SEQ ID NO: 16; and/or (iii) a polypeptide having at least 80% (e.g., at least 85%, 90%, 95% or 100%) sequence identity to MS2-p65-HSF1 polypeptide having SEQ ID NO: 18; (iv) a fusion of a suitable adaptor polypeptide (e.g., least 80% sequence identity to SEQ ID NO: 14) with a suitable activator polypeptide, e.g., p65 and/or HSF1), preferably said third nucleic acid encoding a synergistic activation mediator (SAM) complex and at least one guide RNA (gRNA) comprising at least one aptamer capable of binding at least one co-activator protein (e.g., said co-activator protein comprising one or more of the following: (i) a polypeptide having at least 80% (e.g., at least 85%, 90%, 95% or 100%) sequence identity to the MS2 adaptor polypeptide having SEQ ID NO: 14; (ii) a polypeptide having at least 80% (e.g., at least 85%, 90%, 95% or 100%) sequence identity to MS2-p65 polypeptide having SEQ ID NO: 16; and/or (iii) a polypeptide having at least 80% (e.g., at least 85%, 90%, 95% or 100%) sequence identity to MS2-p65-HSF1 polypeptide having SEQ ID NO: 18; (iv) a fusion of a suitable adaptor polypeptide (e.g., least 80% sequence identity to SEQ ID NO: 14) with a suitable activator polypeptide, e.g., p65 and/or HSF1, most preferably comprising at least two aptamers, each capable of binding at least two co-activator proteins (e.g., said co-activator protein comprising one or more of the following: (i) a polypeptide having at least 80% (e.g., at least 85%, 90%, 95% or 100%) sequence identity to the MS2 adaptor polypeptide having SEQ ID NO: 14; (ii) a polypeptide having at least 80% (e.g., at least 85%, 90%, 95% or 100%) sequence identity to MS2-p65 polypeptide having SEQ ID NO: 16; and/or (iii) a polypeptide having at least 80% (e.g., at least 85%, 90%, 95% or 100%) sequence identity to MS2-p65-HSF1 polypeptide having SEQ ID NO: 18; (iv) a fusion of a suitable adaptor polypeptide (e.g., least 80% sequence identity to SEQ ID NO: 14) with a suitable activator polypeptide, e.g., p65 and/or HSF1,
(iv) optionally, a fourth AAV comprising a fourth nucleic acid encoding a reporter polypeptide, preferably said reporter polypeptide is a fluorescent protein, further preferably said fluorescent protein is a green fluorescence protein;
wherein said first portion of said Cas9 protein devoid of endonuclease activity and said second portion of said Cas9 protein devoid of endonuclease activity, when joined together, form a Cas9 protein devoid of endonuclease activity, preferably said formed Cas9 protein is capable of binding DNA, further preferably said formed Cas9 protein having at least 80% sequence identity to SEQ ID NO: 27 (e.g., Cas9 having UniProtKB Accession Number Q99ZW2).
2. The plurality of item 1, wherein the first portion of the Cas9 protein is the N-terminal lobe of the Cas9 protein and the second portion of the Cas9 protein is the C-terminal lobe of the Cas9 protein.
3. The plurality of any one of the preceding items, wherein:
   (i) the first portion of the Cas9 protein is the N-terminal lobe of the Cas9 protein up to amino acid 573 and the second portion of the Cas9 protein is the C-terminal lobe of the Cas9 protein beginning at amino acid 574; or
   (ii) the first portion of the Cas9 protein is the N-terminal lobe of the Cas9 protein up to amino acid 637 and the second portion of the Cas9 protein is the C-terminal lobe of the Cas9 protein beginning at amino acid 638.
4. The plurality of any one of the preceding items, wherein the first nucleic acid encodes a first portion of the Cas9 protein having a first split-intein and wherein the second nucleic acid encodes a second portion of the Cas9 protein having a second split-intein complementary to the first split-intein, wherein the first portion of the Cas9 protein and the second portion of the Cas9 protein, when joined together, form the Cas9 protein (e.g., by intein-mediated trans-splicing).
5. The plurality of any one of the preceding items, wherein the first nucleic acid encodes a first portion of the Cas9 protein having a Nostoc punctiforme (Npu) strain PCC73102 N-split-intein IntN and wherein the second nucleic acid encodes a second portion of the Cas9 protein having a Nostoc punctiforme (Npu) strain PCC73102 C-split-intein IntC, wherein the first portion of the Cas9 protein and the second portion of the Cas9 protein, when joined together, form the Cas9 protein.
6. The plurality of any one of the preceding items, wherein the SAM complex comprises a MS2 coat protein (e.g., having SEQ ID NO: 14) fused to the p65 subunit of NF-kappaB (e.g., forming SEQ ID NO: 16) and the activation domain of human heat-shock factor 1 (HSF1) (e.g., forming SEQ ID NO: 18).
7. The plurality of any one of the preceding items, wherein a second portion of a Cas9 protein devoid of endonuclease activity is fused to a transcription activation domain, preferably said transcription activation domain having at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) sequence identity with the polypeptide having SEQ ID NO: 20 (VP16).
8. The plurality of any one of the preceding items, wherein the third nucleic acid further encodes a transcription activation domain.
9. The plurality of item 7 or 8, wherein said transcription activation domain comprising at least one VP16 (SEQ ID NO: 20) domain, preferably comprising a quadruple VP16 (VP64) domain.
10. The plurality of any one of the preceding items, wherein the Cas9 is a Type II CRISPR system Cas9.
11. The plurality of any one of the preceding items, wherein the nucleic acid (e.g., first, second, third and/or fourth) is operably linked to a control sequence, preferably operably linked to a suitable promoter (e.g., CbH or GAFP promoter).
12. A composition, kit, expression system or recombinant host cell (e.g., isolated recombinant host cell) comprising the plurality of any one of the preceding items, preferably said composition, kit, expression system or recombinant host cell is pharmaceutical and/or diagnostic composition, kit, expression system or recombinant host cell, further preferably said composition, kit, expression system or recombinant host cell further comprising a reporter, further preferably said reporter is a fluorescent protein, further preferably said fluorescent protein is a green fluorescence protein.
13. The plurality, composition, kit, expression system or recombinant host cell of any one of the preceding items for use as a medicament and/or in therapy.
14. A method for reprogramming and/or modifying a cell, said method comprising:
   a) providing: (i) a cell; and (ii) the plurality, composition, kit, expression system or recombinant host cell of any one of the preceding items;
   b) applying and/or expressing (ii) to/in (i);
   preferably said cell is an astrocyte, further preferably said cell is reprogrammed into a neuron, most preferably said astrocyte is reprogrammed into a neuron.
15. The plurality, composition, kit, expression system or recombinant host cell of any one of the preceding items, for use in one or more of the following methods:
   i) method of treatment, amelioration, prophylaxis and/or diagnostics of a neurodegenerative disease, cancer, cardiovascular disease, metabolic disease, monogenic disorder (e.g., single-gene associated disorder, e.g., Osteogenesis Imperfecta (OGI), Retinoblastoma (RB), Cystic Fibrosis, Thalassemia, Fragile X Syndrome (FXS), Hypophosphatemia, Hemophilia and Ichthyosis) and/or diabetes, preferably said neurodegenerative disease is selected from the group consisting of: Parkinson's disease, Parkinsonism, Parkinson-plus syndrome, Alzheimer's disease, Amyotrophic lateral sclerosis (ALS) and Huntington's disease;
   ii) method for reprogramming and/or modifying a cell, preferably an astrocyte, further preferably into a neuron;
   iii) method for inducing and/or modifying expression of one or more genes of interest (e.g., endogenous, e.g., transcription factors, e.g., one or more of the following: Achaete-scute homolog 1 (Ascl1, e.g., UniProtKB-P50553 or Q02067), LIM homeobox transcription factor 1-alpha (Lmx1a, e.g., UniProtKB-Q8TE12 or Q9JKU8), Nurr1 (e.g., UniProtKB-P43354), preferably or alternatively Achaete-scute homolog 1 (Ascl1, e.g., UniProtKB-P50553 or Q02067), LIM homeobox transcription factor 1-alpha (Lmx1a, e.g., UniProtKB-Q8TE12 or Q9JKU8), Neurogenic differentiation factor 1 (Neurod1, e.g., UniProtKB-Q13562 or Q60867), miRNA218 (e.g., NR_029632, NR 029799.1);
   iv) method for cell-replacement and/or transplantation;
   v) method for somatic reprogramming of a cell; preferably an astrocyte, further preferably into a neuron;
   vi) method for genome and /or transcriptome modification, and/or gene therapy;
   vii) method of screening (e.g., guide RNAs) and/or monitoring gene expression; preferably said composition or kit further comprising a reporter, further preferably said reporter is a fluorescent protein, further preferably said fluorescent protein is a green fluorescence protein;
   viii) method for producing a neuron;
   viii) in a method according to any one of preceding items;
   ix) said method is an in vitro, in vivo or ex vivo method;
   x) in any combination of (i)-(x).
16. Use of the plurality, composition, kit, expression system or recombinant host cell of any one of the preceding items, for one or more of the following:
   i) for reprogramming and/or modifying a cell, preferably an astrocyte, further preferably into a neuron;
   ii) for inducing and/or modifying expression of one or more genes of interest (e.g., endogenous, e.g., transcription factors, e.g., one or more of the following: Achaete-scute homolog 1 (Ascl1 , e.g., UniProtKB-P50553 or Q02067), LIM homeobox transcription factor 1-alpha (Lmx1a, e.g., UniProtKB-Q8TE12 or Q9JKU8), Nurr1 (e.g., UniProtKB-P43354), preferably or alternatively Achaete-scute homolog 1 (Ascl1 , e.g., UniProtKB-P50553 or Q02067), LIM homeobox transcription factor 1-alpha (Lmx1a, e.g., UniProtKB-Q8TE12 or Q9JKU8), Neurogenic differentiation factor 1 (Neurod1, e.g., UniProtKB-Q13562 or Q60867), a ncRNA (non-coding RNA, e.g., miRNA), miRNA218 (e.g., NR_029632, NR_029799.1);
   iii) for cell-replacement and/or transplantation;
   iv) for somatic reprogramming of a cell; preferably said cell is selected from the group consisting of: an astrocyte, cardiomyocyte, adipocyte, muscle cell, osteoclast, osteoblast, osteocytes, a blood cell (e.g., white blood cell), a skin cell, a stem cell, further preferably said astrocyte is reprogrammed into a neuron;
   v) for genome modification and/or gene therapy;
   vi) for producing a neuron;
   vii) as a medicament and/or in therapy;
   viii) for treatment, amelioration, prophylaxis and/or diagnostics of a degenerative disease (e.g., neurodegenerative disease), cancer, cardiovascular disease, metabolic disease, monogenic disorder (e.g., single-gene associated disorder, e.g., Osteogenesis Imperfecta (OGI), Retinoblastoma (RB), Cystic Fibrosis, Thalassemia, Fragile X Syndrome (FXS), Hypophosphatemia, Hemophilia and Ichthyosis) and/or diabetes, preferably said neurodegenerative disease is selected from the group consisting of: Parkinson's disease, Parkinsonism, Parkinson-plus syndrome, Alzheimer's disease, Amyotrophic lateral sclerosis (ALS) and Huntington's disease;
   ix) in a method according to any one of preceding items;
   x) for in vitro, in vivo or ex vivo use;
   xi) in any combination of (i)-(x).

The invention is further illustrated by the following examples (e.g., as illustrated in FIGS. 1-44), however, without being limited to the example or by any specific embodiment of the examples.

### Examples of the invention

### Methods

### Generation of the Split-dCas9

The nuclease inactivating point mutations D10A and N863A were introduced into the plasmids pAAV_crTLR#1_Nv1 and pAAV_crTLR#1_Cv1 from Truong et al. using QuikChange II Site Directed Mutagenesis Kit (Agilent Technologies, 200523, USA) (Truong et al., 2015). miRNA218 cloning was performed according to Rivetti di Val Cervo et al (Rivetti di Val Cervo et al., 2017).

### Animals and Surgery

For the analysis the B6.Cg-Tg(Gfap-cre)77.6Mvs/2J (Gfap-Cre) was purchased from Jackson Laboratories (#024098). The Rosa26-dCas-activator mouse line was generated using CRISPR/Cas9-based gene editing by microinjection into one cell embryos. Adult mice (3-4 month) received a unilateral injection of 6-hydroxydopamine-HCl (6-0HDA-HCl) (Sigma-Aldrich, H4381, USA) into the left medial forebrain bundle (AP −1.2, ML +1, DV −4.9). Two weeks after toxin injection, high titer recombinant adeno-associated virus was applied into the ipsilateral striatum (AP +1, ML +2.1, DV −3.5). Stereotaxic coordinates (millimeters) are relative to bregma.

### Behaviour Analysis

To assess voluntary movement mice were tested on an automated, video-based gait analysis system, the CatWalk XT (Noldus, Wageningen, The Netherlands). For the vertical pole test the mice were placed facing upwards onto a wooden, rough-surfaced pole and tested for the time they needed to turn downwards. For the drug-induced circling behavior mice received an intraperitoneal injection of 5 mg/kg amphetamine before being placed into a transparent cylinder (diameter 12.5 cm, ehight 30 cm). After 15 min of habituation, they were monitored for 45 min and automated 90° body rotation counts were counted using Ethovision software (Ethovision XT 14, Noldus, Wageningen, The Netherlands).

### Molecular Cloning

### Polymerase Chain Reaction

PCRs are performed using the Q5 High-Fidelity 2× Master Mix (NEB, M0492S, USA). For the amplification of GC-rich regions the KAPA HiFi HotStart PCR Kit (Kapa Biosystems, KK2501, Swiss) was used. For colony PCR and genotyping reactions VWR Red Taq DNA Polymerase Master Mix (VWR, 733-2131, USA) was deployed. For STAgR cloning the Phusion High-Fidelity DNA Polymerase (Thermo Fisher, F530S, USA) was applied. Site-directed mutagenesis was performed using QuikChange II Site Directed Mutagenesis Kit (Agilent Technologies, 200523, USA). All reactions were performed according to manufacturer's instructions.

If the PCR product was further employed in cloning steps it was either PCR purified using QlAquick PCR purification kit (Quiagen, 28104, Netherlands), or gel purified followed by a gel purification step using QlAquick gel extraction kit (Quiagen, 28115, Netherlands), both reactions were performed according to manufacturer's instructions.

### Enzymatic Digest

Restriction enzymes from New England Biolabs were used according to manufacturer's instructions. For plasmid digest 500 ng to 1 μg of DNA was digested and subsequently gel purified using QlAquick gel extraction kit (Quiagen, 28115, Netherlands). For the digest of PCR products 500 ng of DNA was used followed by a PCR purification using QlAquick PCR purification kit (Quiagen, 28104, Netherlands), both reactions were performed according to manufacturer's instructions.

### DNA Ligation

DNA fragments were ligated using T4 DNA Ligase (NEB, M0202S, USA) using 20 ng of vector DNA and a molar ratio of vector/insert of 1/3, reaction was performed for 20 minutes at room temperature. For the ligation of multiple PCR fragments Gibson assembly was performed using NEBuilder0 HiFi DNA Assembly Master Mix (NEB, E2621S, USA), fragments were used in an equimolar ratio and reaction was performed for 1 h at 50° C.

sgRNA Design and Cloning

All sgRNAs were designed using the online tool benchling.com. sgRNAs were targeted to the region -250 bp to the transcriptional start site of the target gene. Two sgRNAs were used per gene (see corresponding DNA binding sites below). Multiplexed sgRNA cloning was performed using the string assembly sgRNA cloning strategy (STAgR) (Breunig et al., 2018).

| | | | SEQ |
|---|---|---|---|
| Name | Gene | Sequence 5′-3′ | ID NOS: |
| Ascl1-1 | Ascl1 | GGGAGCCGCTCGCTGCAGCAGCG | 28 |
| Ascl1-2 | Ascl1 | GGGGCTGAATGGAGAGTTTGCA | 29 |
| Lmx1a-1 | Lmx1a | GGGAGCAAAGGAGTCGCCTTG | 30 |
| Lmx1a-2 | Lmx1a | GAATGCATCCAAGAGTGAACC | 31 |
| Nr4a2-1 | Nr4a2 | GGCGGTGGGTCATTGTTTCCG | 32 |
| Nr4a2-2 | Nr4a2 | GTGCCAGTGACGCCGGCCTGG | 33 |
| NeuroD1-1 | NeuroD1 | GGTTCTGGGAGGGGTGAATGA | 34 |
| NeuroD1-2 | NeuroD1 | GGCCATATGGCGCATGCCGGGG | 35 |
| Neurog2-1 | Neurog2 | ATAAGCTGGGGAGGGAGAGC | 36 |
| Neurog2-2 | Neurog2 | AAACAATCAGATCTGCCCCG | 37 |
| PITX3-1 | PITX3 | ATTCACHTATGGCAACCCA | 38 |
| PITX3-2 | PITX3 | GCTAGCCTGGGAGAGCCCAG | 39 |
| FoxA2-I | FoxA2 | GAAAGTAACCTTGAAACACCG | 40 |
| FoxA2-2 | FoxA2 | GGGTAGCCAGAAAGAGGACTG | 41 |
| MyoD1-1 | MyoD1 | CCAATAGGAGTGTAGTAGGG | 42 |
| MyoD1-2 | MyoD1 | GAGAGACTGGCAGCCATACG | 43 |
| Pou5f1-1 | Pou5f1 | ATCTGCCTGTGTCTTCCAGA | 44 |
| Pou5f1-2 | Pou5f1 | TGTCCGGTGACCCAAGGCAG | 45 |

### Transformation and Plasmid Purification

Chemically competent DH5a or NEB stable (plasmids for AAV production) bacteria were used for transformation. After a heat-shock was performed bacteria were spread on agar plates containing the suitable selection marker. Plates were incubated over night at 37° C. and single colonies were picked for further analysis.

For plasmid purification Plasmid Mini Kit (Qiagen, 12123, Netherlands) or EndoFree Plasmid Maxi Kit (Qiagen, 12163, Netherlands) was used according to manufacturer's instructions.

### Cell Culture

All cells are incubated at 37° C. with 7.5% CO2. Neuro2A cell line was purchased from ATCC (ATCC, CCL-131, USA). Cells are cultures in DMEM/F12 GlutaMAXTM-I medium with 10% FCS.

### Isolation of Primary Cortical Astrocytes

Primary cortical astrocytes were obtained from postnatal (P5-P6) mice following a protocol adapted from Heinrich et al.(Heinrich et al., 2011). After tissue dissection, the cortices were dissociated and purified using the Adult Brain Dissociation Kit from Miltenyi (Miltenyi, 130107677, Germany). Instead of using the gentleMACS Octo Dissociator the tissue was kept in the enzyme mixture for 30 minutes, every 10 minutes the mixture was pipetted up and down (5 times) using a 10 mL serological pipette for tissue dissociation. Subsequently the protocol was performed according to manufacturer's instructions without conducting the red blood cell removal. For the purification of astrocytes the cortical cell mixture was separated using the Anti-ACSA-2 MicroMead Kit (Miltenyi, 130097678, Germany). As soon as the cells reach a confluency of ˜80% (day 7-10), 300.000 cells were seeded per 6 well.

### Lipofection

Astrocytes were transfected using Lipofectamine 2000 (Invitrogen, 11668, USA) according to manufacturer's instructions. 30 minutes prior to the lipofection cells are equilibrated in 1.5 ml OptiMEM with 10% glutamine. 3.6 μg of DNA is transfected per 6 well using a DNA/lipofectamine ratio of 1/3. 4 h later the transfection media is removed and exchanged by conditioned astrocyte media. 48 hours after the transfection the RNA is isolated, respectively cells are fixed using 4% paraformaldehyde for immunocytochemistry.

### FACS Sorting

Astrocytes were trypsinated for 5 minutes using 0.05% trypsin-EDTA (Thermo Fisher, 25300054, USA), reaction was stopped with PBS pH 7.4 with 5% fetal bovine serum (Thermo Fisher, A2153, USA). After centrifugation cells were resuspended in PBS pH 7.4 with 0.5% fetal bovine serum and kept on ice until further processing. The green fluorescent protein was co-transfected in order to enrich cells that were successfully transfected. Green cells were enriched with the BD FACSaria II controlled with the BD FACSDiva Software Version 6.1.3 (BD Biosciences, USA), cells were collected and further processed for RNA isolation.

RNA Isolation, cDNA Preparation

Given the low transfection efficiency, cells are sorted using the FACSARIA III (Biosciences) with a 100 μm nozzle according to GFP signal, expressed from a co-transfected plasmid. RNA is isolated using PicoPure RNA Isolation Kit (Invitrogen, KIT0204, USA). cDNA is produced using SuperScript VILO cDNA Synthesis Kit (Thermo Fisher, 11754050, USA).

Real Time qPCR qPCR is performed using TaqMan Universal Master Mix (Thermo Fisher, 4304437, USA) and TaqMan probes, all probes are listed herein. Reaction was performed according to manufacturer's instructions. RT-qPCR was carried out using an ABI Prism 7900 HT Real-Time PCR System and SDS 2.4.1 software.

| Gene | TaqMan probe (Assay ID) |
|---|---|
| Ascl1/Mash1 | Mm03058063_m1 |
| Lmx1a | Mm00473947_m1 |
| Nr4a2/NR4A2 | Mm00443060_m1 |
| NeuroD1 | Mm01280117_m1 |
| Ngn2 | Mm00437603_g1 |
| PITX3 | Mm01194166_g1 |
| FoxA2 | Mm00839704_mH |
| MyoD1 | Mm00440387_m1 |
| Pou5fl | Mm00658129_gH |
| B-Actin | Mm00607939_s1 |

### Immunocytochemistry

Cells were fixed using 4% paraformaldehyde. Primary and secondary antibodies were diluted in PBS containing 1% bovine serum albumin and 0.5% Triton X-100. Primary antibody was incubated overnight at 4° C., secondary antibody was for one hour at room temperature. Primary antibodies: mouse-anti-ASCL1 1:1000 (BD Bioscience, 556604, USA), rabbit-anti-LMX1A 1:2000 (Merck-Millipore, ab10533, Germany), mouse-anti-Nr4a2 1:2000 (Santa Cruz, se-376984, USA), rabbit-anti-Flagtag 1:1000 (Sigma, F1804, USA), rabbit-anti-MAP2 1:1000 (Merck-Millipore, AB5622, Germany). Secondary antibodies: Donkey anti-mouse IgG Alexa Fluor 594 1:500 (Thermo Fisher Scientific, A-21203, Germany), donkey anti-rabbit IgG Alexa Fluor 594 1:500 (Thermo Fisher Scientific, A-21207, Germany). Coverslips were mounted onto glass slides using Aqua-Poly/Mount.

### Lentivirus Production

Production and titer determination of replication incompetent, self-inactivating lentiviruses was performed as described before (Theodorou et al., 2015).

### Western Blot

Primary antibodies were diluted in TBS-T containing 0.5% milk powder and incubated over night at 4° C. Primary antibodies: rabbit-anti-HA tag (C29F4) 1:500 (Cell Signaling, 3724, USA), mouse-anti-β-Actin 1:10000 (GeneTex, GTX26276, USA), anti-mouse-N-Cas9 1:500 (Epigentek, A-9000, USA), anti-mouse-C-Cas9 1:1000 (Novus biologicals, NBP2-52398SS, USA), anti-rabbit-P2A 1:1000 (Sigma-Aldrich, ABS31, USA). Secondary antibodies were diluted in TBS-T containing 5% milk powder and incubated for 1 hour at room temperature. Secondary antibodies: Goat anti-rabbit IgG HRPO 1:5000 (Dianova, 111-035-003, USA), goat anti-mouse IgG HRPO 1:5000 (Dianova, 115-035-003, USA).

### Animals

For the analysis the B6.Cg-Tg(Gfap-cre)77.6Mvs/2J (GFAP-Cre) was purchased from Jackson Laboratories (024098), the line was further bred on a B6N background. The Rosa26-dCas-activator mouse line (dCAM) was produced on a B6N background. For the analysis littermates of the B6.Cg-Tg(Gfap-cre)77.6Mvs/2J x dCAM/N line was used. When crossing the B6.Cg-Tg(Gfap-cre)77.6Mvs/2J line with transgenic animal carrying LoxP cassettes, it was payed attention to only breed female Cre mice, as it is known for this line to have Cre expression in the male germline.

### Generation of CRISPR-Activator Mouse Line via Microinjection of One-Cell Embryo

The Rosa26-dCas-activator mouse line was generated using CRISPR/Cas9-based gene editing by microinjection into one cell embryos. For this, a gene specific guide RNA (Rosa26_gRNA 5′-ACTCCAGTCTTTCTAGAAGA-3′) was used as in vitro transcribed single gRNA (EnGen® sgRNA Synthesis Kit, NEB, E3322, USA). Prior to pronuclear injection, gRNA (25 ng/μl) and targeting vector (50 ng/μl) were diluted in microinjection buffer (10 mM Tris, 0.1 mM EDTA, pH 7.2) together with recombinant Cas9 protein (50ng/μl, IDT, Coralville, USA) and incubated for 10 min at room temperature and 10 minutes at 37° C. to form the active ribonucleoprotein (RNP) complex. One-cell embryos were obtained by mating of C57BL/6N males (obtained from Charles River, Sulzbach, Germany) with C57BL/6N females superovulated with 5 units PMSG (Pregnant Mare's Serum Gonadotropin) and 5 units HCG (Human Chorionic Gonadotropin). For microinjections, one-cell embryos were injected into the larger pronucleus. Following injection, zygotes were transferred into pseudo-pregnant CD1 female mice to obtain live pups. All mice showed normal development and appeared healthy. Handling of the animals was performed in accordance to institutional guidelines and approved by the animal welfare committee of the government of upper Bavaria. The mice were housed in standard cages in a specific pathogen-free facility on a 12 h light/dark cycle with ad libitum access to food and water. Analysis of gene editing events was performed on genomic DNA isolated from ear biopsies of founder mice and F1 progeny, using the Wizard Genomic DNA Purification Kit (Promega, A1120, Germany) following the manufacturer's instructions.

### Animal Housing

Animal housing and handling protocols were approved by the committee for the Care and Use of Laboratory animals of the Government of Upper Bavaria (Germany) and were carried out in accordance with the European Communities' Council Directive 2010/63/EU. During the work, all efforts were made to minimize animal suffering. All mouse lines were kept in a controlled pathogen free (SPF) hygiene standard environment on a 12 h light/dark cycle. The mice had access to ad libitum standard feed and water always. All tests were approved for the ethical treatment of animals by the Government of Upper Bavaria.

### 6-OHDA Lesion

Adult (3-4 months) mice were chosen for dopamine depletion of the left striatum, mice received a unilateral injection of 6-hydroxydopamine-HCl (6-OHDA-HCl) (Sigma-Aldrich, H4381, USA) into the left medial forebrain bundle (MFB). All animals receive intraperitoneal injection of Medetomidin (0.5 mg/kg), Midazolam (5mg/kg), Fentanyl (0.05 mg/kg) (MMF) as anesthesia. The mouse received pre-emptive Metamizol (200 mg/kg s.c.) and a local subcutaneous injection of 2% Lidocain. The animal was positioned into the stereotactic frame containing an integrated warming base (Stoelting, 51730D, USA) to maintain normothermia. 6-OHDA-HCl was dissolved in 0.2% ascorbic acid (Sigma-Aldrich, A4403, USA) in saline at a concentration of 2 μg/pl of free-base 6-0HDA-HCl. Each mouse was injected 1.5 μl (0.2 μl/min) of solution into the left MFB according to the following coordinates: anteroposterior (AP) -1.2, mediolateral (ML) +1, dorsoventral (DV) -4.9 (all millimeters relative to bregma) with flat skull position. The needle was left in place for 3 minutes after the injection to allow the toxin to diffuse before slow withdrawal of the capillary. Mice were woken up from anesthesia by the subcutaneous injection of Atipamezol (2.5 mg/kg) and Flumazenil (0.5 mg/kg). Mice were left for recovery for 2 weeks before experimentation.

### Stereotactic Injection

The dopamine depleted animals were injected into the ipsilateral striatum with high titer recombinant adeno-associated virus (AAV). Mice were anesthetized with MMF and received pre-emptive pain treatment as for the 6-OHDA-HCl injection, subsequently they were positioned into the stereotactic frame with flat skull position. Each mouse received 1 μl rAAV2/5 (0.2 μl/min) into the left dorsal striatum according to the following coordinates: AP +1, ML +2.1, DV −3.5 (all millimeters relative to bregma). The needle was left in place for 3 minutes after the injection to allow the virus to diffuse before slow withdrawal of the capillary. Mice were woken up from anesthesia by the subcutaneous injection of Atipamezol (2.5 mg/kg) and Flumazenil (0.5 mg/kg).

rAAV Production

High-titer preparations of rAAV2/5 were produced based on the protocol of Zolotukhin and colleagues (Zolotukhin et al., 1999) with minor modifications. In brief, HEK 293T cells were transfected with the CaPO4 precipitation method, the plasmids pRC5, Ad helper and pAAV were applied in an equimolar ratio. After 72 h, cell pellet was harvested with AAV release solution, 50 U/ml benzonase was added, then solution was incubated for 2 h at 37° C. Cells were frozen and thawed in liquid nitrogen to allow rAAV release. Purification of rAAV vector was done with iodixanol densities gradient (consisting of 15, 25, 40 and 56% iodixanol), followed by gradient spinning at 50.000 rpm for 2 h 17 min at 22° C. in a Ti70 rotor (Beckman, Fullerton, CA, USA). rAAV was collected at 40% iodixanol with a 5 ml syringe. Virus was dialyzed (Slide-A-Lyzer 10.000 MWCO 5m1) in buffer A overnight to remove iodixanol. Anion exchange chromatography column HiTrap Q FF sepharose column and Superloop were connected with the AKTAprime plus chromatography system to collect the eluted fraction. To measure rAAV concentration, the eluted fraction was spun and washed once in PBS-MK Pluronic-F68 buffer with a Millipore 30K MWCO 6 ml filter unit. rAAVs were stored in a glass vial tube at 4° C. rAAVs were titered by SYBR Green qPCR with GFP or SV40 primer (D′Costa et al., 2016). Usual titer was 3 x 1014 to 5 x 1015 GC/ml.

### Immunohistochemistry

For histological analysis the mice were asphyxiated with CO2 and perfused transcardially with 4% ice-cold paraformaldehyde (PFA) (Sigma-Aldrich, P6148, USA) in 0.1 M PBS with pH 7.4. After dissection the brain was post-fixed in PFA overnight at 4° C. followed by storage in 30% sucrose for minimum 24 hours at 4° C. Brains were cut coronal into 40 μm thick serial sections on a cryostat (Thermo Fisher Scientific, HM 560 Kryostat, Microm, Germany). Free floating sections were stored at 4° C. in cyro protection solution (50% PBS pH 7.4, 25% ethylene glycol (Carl Roth, 2441, Germany), 25% glycerol (Sigma-Aldrich, G9012, USA)) until further processing.

In general sections were blocked in PBS pH 7.4 with 2% fetal bovine serum (Thermo Fisher, A2153, USA) and 0.1% Triton X-100 (Sigma-Aldrich, T9284, USA) for 2 hours. Subsequently, brain slices were incubated over night at 4° C. in primary antibody diluted in blocking solution. Sections were three times washed for 15 minutes with PBS pH 7.4 before incubated with secondary antibody diluted in PBS pH 7.4 containing 0.1% Triton X-100 (Sigma-Aldrich, T9284, USA) for one hour at room temperature. Slices were washed with 100 ng/mL DAPI-PBS solution pH 7.4 (Sigma-Aldrich, D8417, USA) for 5 minutes, followed by three 15 minutes washes with PBS pH 7.4. Slices were mounted on coverslips using Aqua-Poly/Mount (Polysciences, 18606, USA). For the NeuN staining the sections were undertaken an antigen retrieval protocol. In short, the sections were incubated in 0.01 M Na-citrate buffer pH 6 at 80° C. for 45 minutes and allowed to cool down to room temperature per se. Subsequently, brain slices were blocked in 3% milk solution containing 0.3% Triton X-100 for 2 hours. Sections are incubated overnight at 4° C. in primary antibody diluted in blocking solution. Sections are washed three times for 1 hour in PBS pH 7.4 containing 0.3% Triton X-100 and incubated overnight at 4° C. in secondary antibody diluted in blocking solution. Slices were washed with 100 ng/mL DAPI-PBS solution pH 7.4 (Sigma-Aldrich, D8417, USA) for 5 minutes, followed by three 15 minutes washes with PBS pH 7.4. Slices were mounted on coverslips using Aqua-Poly/Mount (Polysciences, 18606, USA). Primary antibodies: rabbit-anti-tyrosine hydroxylase 1:500 (Pel-Freeze, P40101, USA), mouse-anti-NeuN 1:1000 (Abcam, ab104224, USA), anti-chicken-GFP 1:1000 (Abcam, ab13970, USA), anti-rabbit-GFAP 1:1000 (Abcam,ab7260, USA), anti-mouse-Parvalbumin 1:1000 (Sigma-Aldrich, P3088, USA), anti-rabbit-calretinin 1:1000 (Swant, CR7697, Switzerland), anti-goat-CHAT 1:100 (Merck-Millipore, AB144P, Germany), anti-rabbit-Gad65/67 1:500 (Abcam, ab49832, USA), anti-mouse-vGLUT1 1:1000 (Atlas, AMAb91041, USA), anti-rabbit-DARPP32 1:500 (Abcam, ab40801, USA), anti-rabbit-MAP2 1:500 (Merck-Millipore, ab5622, Germany), anti-rabbit-TUJ1 1:500 (Abcam, ab18207, USA). Secondary antibodies: Donkey anti-mouse IgG Alexa Fluor 594 1:500 (Thermo Fisher Scientific, A-21203, Germany), donkey anti-rabbit IgG Alexa Fluor 594 1:500 (Thermo Fisher Scientific, A-21207, Germany), donkey anti-chicken IgY Alexa Fluor 488 1:250 (Dianova, 703-546-155, Germany).

### Image Acquisition

All images were acquired on a confocal laser scanning (Zeiss LSM880) microscope, if not differently indicated.

### Cell Counting

All stereological quantifications were performed using the Stereoinvestigator Zeiss Imager M2 with the software version 2019.1.3. The dorsal striatum of at least three animals was analyzed for quantification. Regions close to the subventricular zone were excluded form counting.

### Behaviour Analysis

Thirteen weeks after rAAV intracerebral injection mice underwent gait analysis.

### Catwalk

Mice were tested on an automated, video-based gait analysis system, the CatWalk XT (Noldus, Wageningen, The Netherlands). The animals walk over an elevated glass walkway (width 8 cm, length 100 cm) enclosed by plexiglas walls (height 14 cm) in a dark room. A camera (Pulnix Camera RM-765) situated below the middle of the walkway tracked the illuminated footprints, which were later analyzed with the CatWalk software Version 7.1. The software automatically calculates a wide number of parameters in several categories which describe gait in spatial and temporal aspects. For a more detailed description see HOlter et al. and Zimprich et al.(1-161ter and Glasl, 2012; Zimprich et al., 2018).

Drug-induced Rotation Analyses

The mice were placed individually in plexiglas cylinders (diameter 12.5 cm, height 30 cm). Experiments were recorded from a ventral plane view, videos were analyzed with the automated 90° body rotation counts using Ethovision software (Ethovision XT 14, Netherlands). Mice were allowed to habituate for 15 min before monitoring for 45 min. Amphetamine was dissolved in saline at a concentration of 0.5 mg/mL, each mouse received an intraperitoneal injection of 5 mg/kg before being placed into the cylinder.

### Vertical Pole Test

Mice were placed facing upwards onto a wooden, rough-surfaced pole (length 50 cm, diameter 1 cm) with a square base plate. Mice were tested for the time they need to turn downwards (latency time) and the total time they need to reach the base of the pole (total time). Right before the test trials, the mice were trained in small groups with less than ten animals. Each mouse was coached three to five times before moving on to the next one. Then three test trials were performed with each mouse in the same sequential order, so that the time interval between training and testing was the same for each individual.

### Electrophysiology

### Preparation of Brain Slices

Acute 220 μm thick brain coronal slices containing the dorsal striatum were cut on a vibratome (Leica VT1200, Germany) in a bubbled (95% O2/5% CO2) standard ice-cold artificial cerebrospinal fluid (ACSF) containing (in mM): 126 NaCl, 2.5 KCl, 1.2 MgCl2, 2.4 CaCl2, 1.2 NaH2PO4, 21.4 NaHCO3, 11.1 glucose, complemented from slicing only with (in mM): 3 kynurenic acid, 26.2 NaHCO3, 225 sucrose, 1.25 glucose and 4.9 MgCl2. Slices were then transferred to a chamber containing standard ACSF oxygenated with 95% O2/5% CO2 at 35° C. for 15 min and subsequently maintained at room temperature for at least another 15 min prior to use.

### Whole-Cell Recordings

Dorsal striatal “reprogrammed” cells (either neurons or glia) were visualized with a 20x/1.0NA WI objective, 4x post-magnification, under video microscope (Olympus BX51WI, Germany) coupled with infrared gradient contrast and epifluorescence. Whole-cell patch-clamp recordings in current clamp mode were acquired from the somata of fluorescent cells with a Multiclamp 700B amplifier (Molecular Devices, Foster City, CA), digitized at 10 kHz and Bessel filtered at 4kHz. Pipettes (4-6 m0) were filled with an intracellular solution containing (in mM): 100 K-gluconate, 20 KCl, 4 Mg-ATP, 0.3 Na-GTP, 10 Na2-Phosphocreatine, 10 Hepes, (pH 7.3, 290 mOsm). All recordings were carried out at 35° C. and slices continually superfused with oxygenated (95% 02/5% CO2) ACSF. Passive membrane properties were assessed by injecting 500 ms depolarizing current steps. Putative spontaneous postsynaptic potential were recorded with the same internal solution in voltage clamp mode while the cell being held at -70 mV. Data were analyzed with custom-written routines in IgorPro.

### Single Cell Analysis

### Tissue Dissociation

Cells were dissected from mouse striatum (n=2) and dissociated into single cell suspension using the papain kit (Worthington) according to manufacturer's instructions. Incubation with dissociating enzyme was performed for 90 min.

### Library Preparation and Sequencing

Single cell suspensions were loaded onto 10× Genomics Single Cell 3′Chips together with the reverse transcription mastermix according to manufacturer's instructions for the Chromium Single Cell 3library & Gel Bead Kit v2 (PN-120237, 10× Genomics) to generate single cell gel beads in emulsion (GEMs). cDNA synthesis was done according to 10×Genomics guidelines. Libraries were pooled and sequenced on a NovaSeq6000 (Illumina) according to the Chromium Single Cell v.2 specifications and with an average read depth of 50,000 aligned reads per cell. Sequencing was performed in the genome analysis center of the Helmholtz Center Munich.

### Alignment and Data Analysis

Transcriptome alignment of single cell data was done using Cell Ranger 3.1.0 against a modified version of the mouse transcriptome GrCm38 (Ensembl Release 99) that included both GFP and Cre sequences. Quality Control (QC) of mapped cells was done using recommendations by Luecken et al.(Luecken and Theis, 2019), selecting 3,899 cells with at least 800 reads and 250 detected genes. Normalization and log transformation was performed using the counts per million (CPM) strategy with a target count depth of 10,000 using SCANPY's (Wolf et al., 2018) normalize_total and log1p functions. Highly variable gene selection was performed via the function highly_variable_genes using the Seurat49 flavour with default parametrization, obtaining 4,274 HVGs in at least one experimental group. Following cell count normalization experimental groups were integrated with Scanorama (Hie et al., 2019). Unsupervised clustering of cells was done using the Leiden algorithm (Traag et al., 2019b) as implemented in SCANPY and with resolution parameter of 0.05. This allowed classification and counting of nine main cell types based on marker genes selected using t-test between the normalized counts of each marker gene in a cell type against all others (function rank_genes_groups in SCANPY). 1,110 cells assigned to astrocytic and neuronal cell types were subclustered into four groups using Leiden with a resolution of 0.30. Marker genes in these four groups were detected using t-test between each group against the other three. Detection of cells positive for GFP, Cre and other marker genes was done using as criteria any cell with normalized counts greater than zero. Visualization of cell groups is done using Uniform Manifold Approximation and Projection (UMAP) (Melville et al., 2018), as implemented in SCANPY.

### Statistics

Statistical analysis was performed using Graphpad Prism 7 software. If not differently indicated, at least three biological replicates were analyzed. The normality of the distribution of data points was verified using Shapiro-Wilk test. Data was analyzed using either an unpaired t-test or a multiple comparison ANOVA, followed by a posthoc Tukey's multiple comparisons test. When normality tests did not indicate normal distribution, non-parametric Kruskall-Wallis test was performed. Asterisks are assigned as followed * p<0.5, ** p<0.01, *** p<0.001, **** p<0.0001.

### Example 1: Results: Generation of the Conditional Rosa26 knock-in dCas9 Activator Mouse (dCAM)

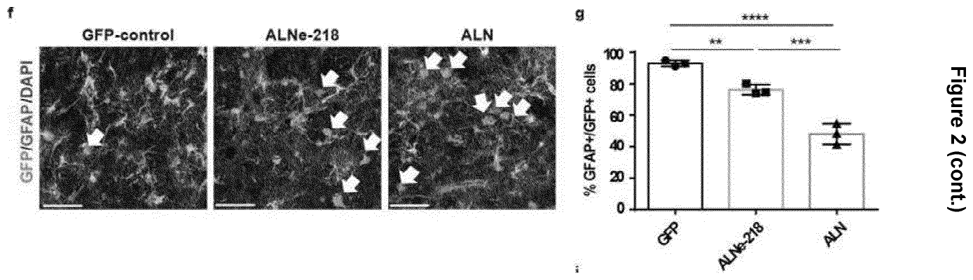

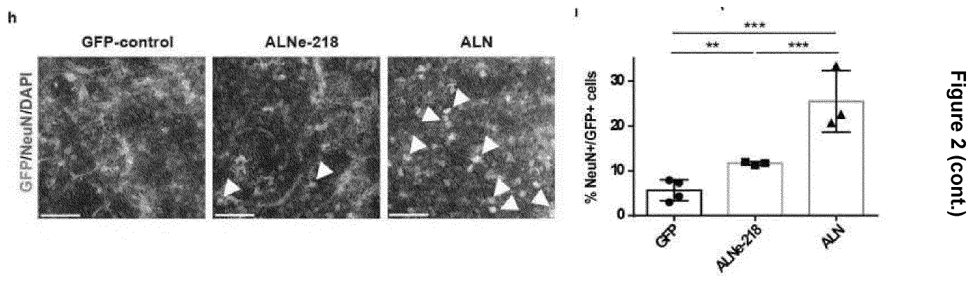

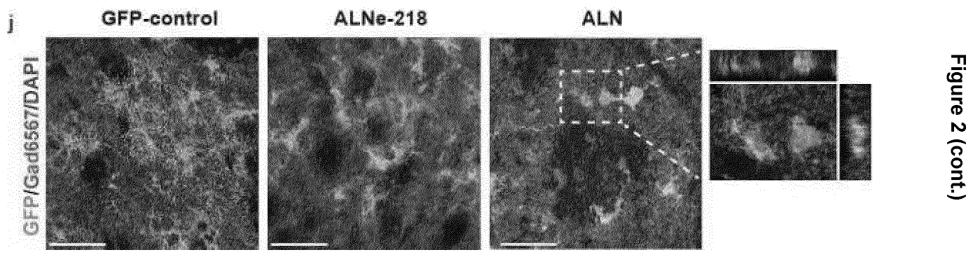

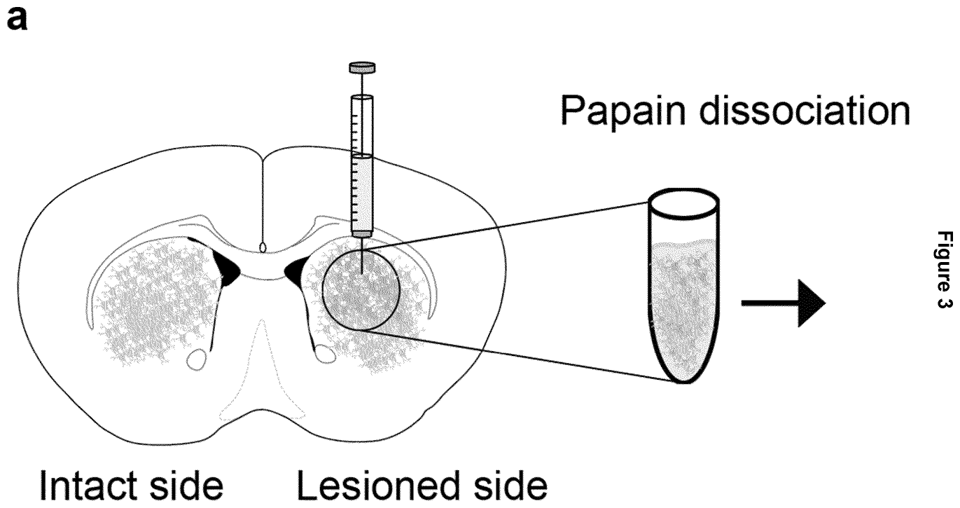

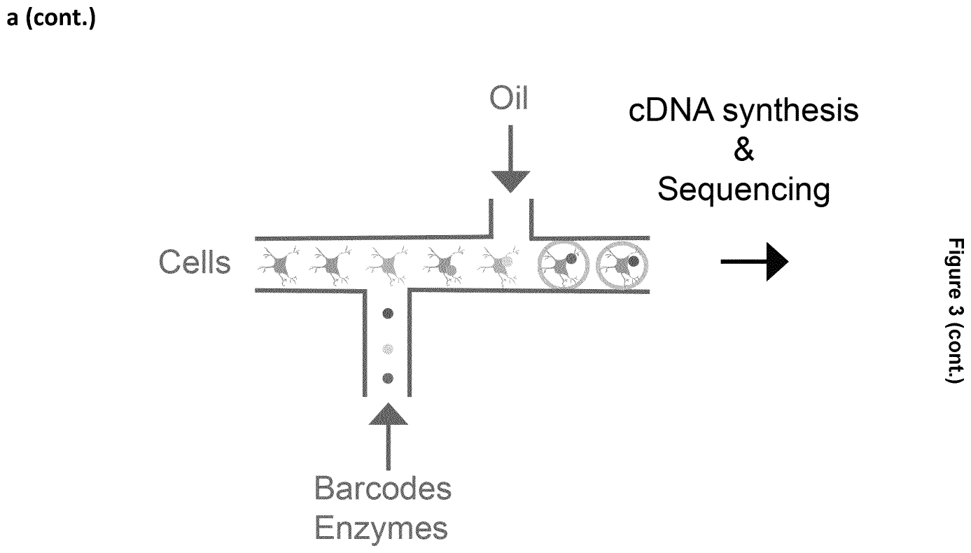

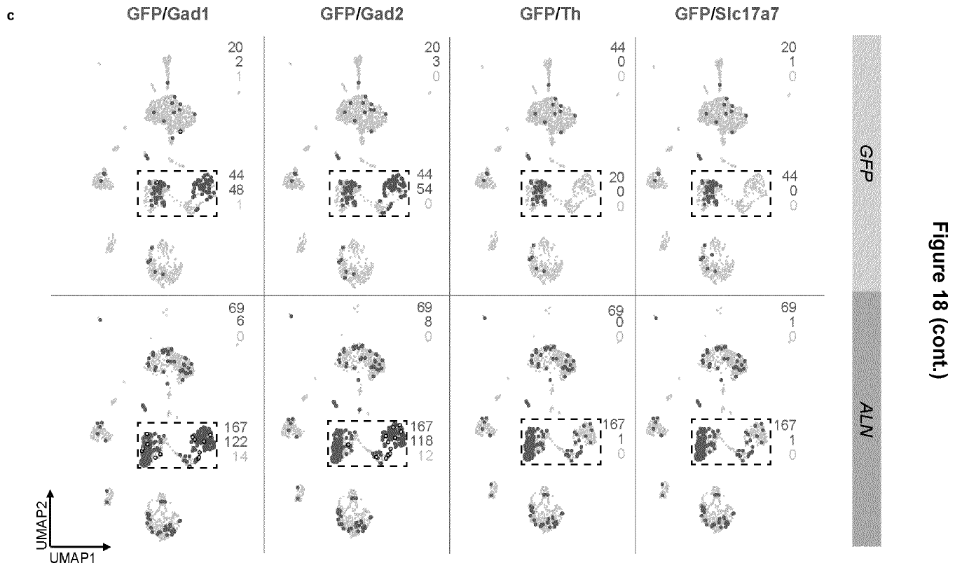

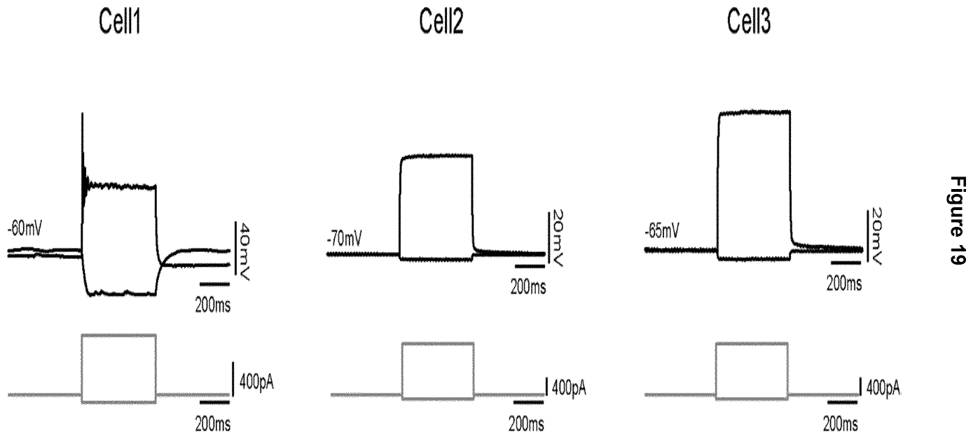

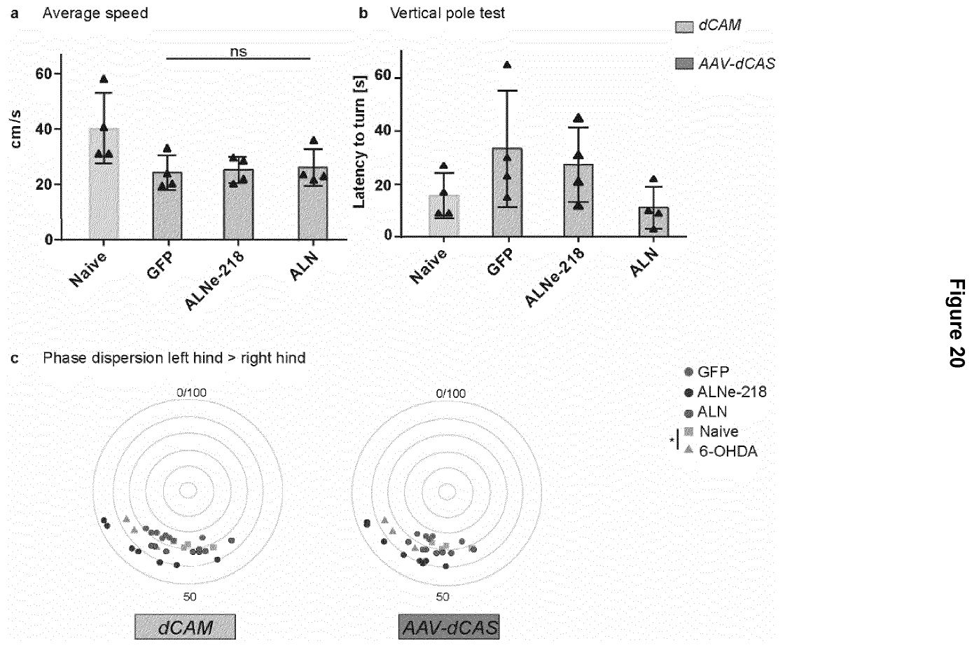

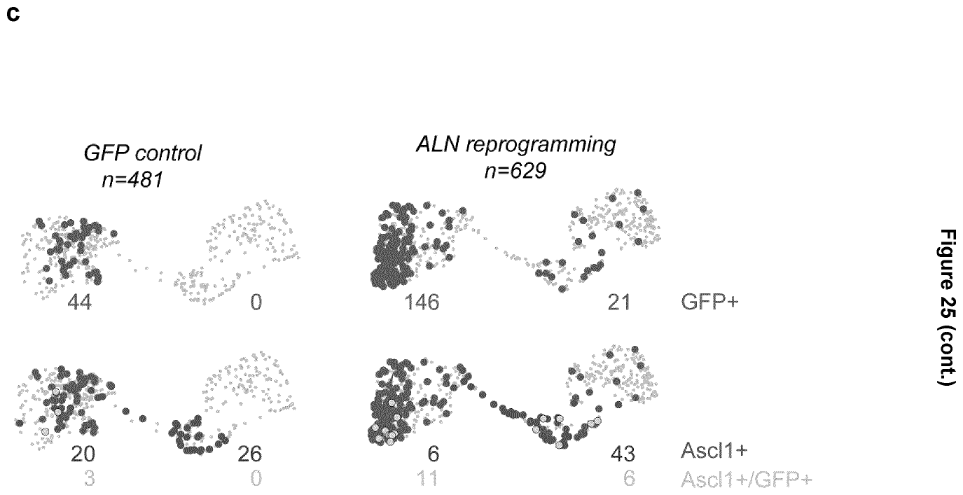

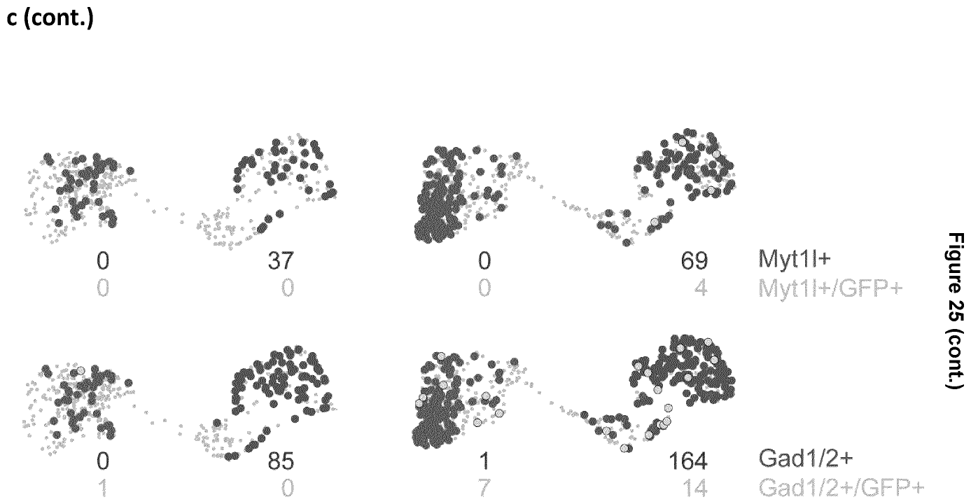

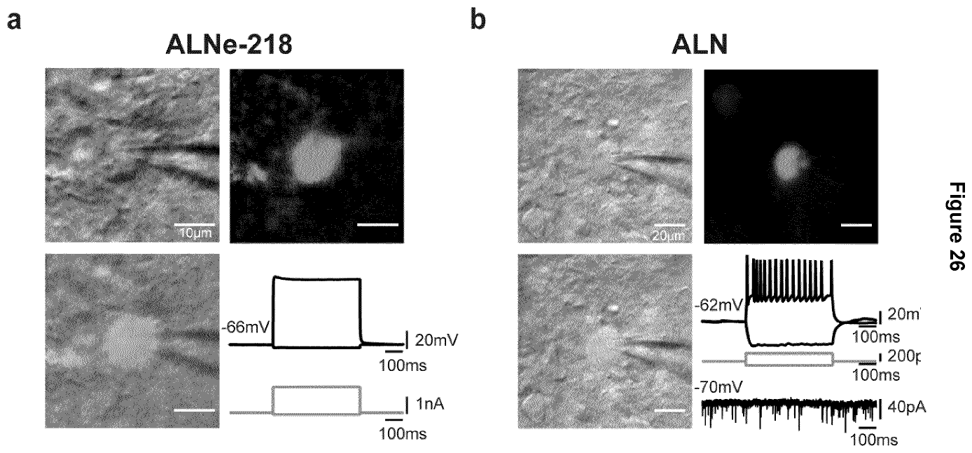

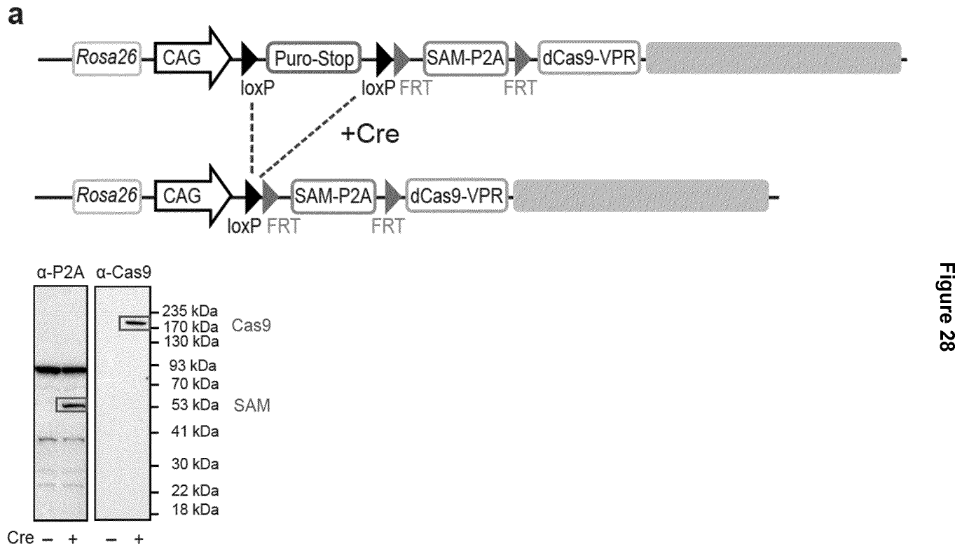

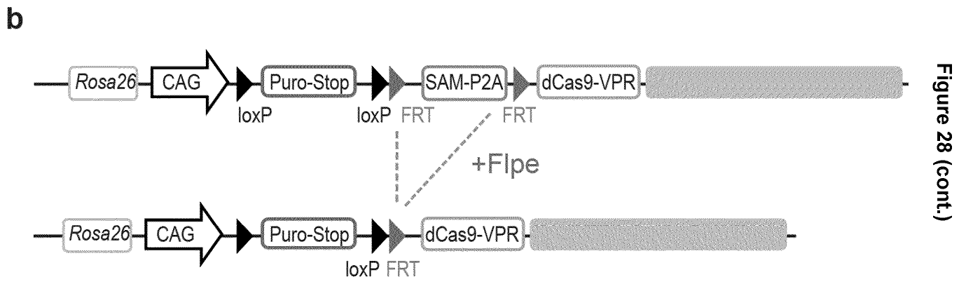

To facilitate the comprehensive and efficient application of CRISPR/Cas9 activation (CRISPRa) in vivo, we generated a dCas9-activator knock-in mouse line in the safe harbor locus Gt(ROSA)26Sor, combining two previously described activation systems dCas9-VPR and SAM (synergistic activation mediator) (Chavez et al., 2015; Konermann et al., 2015) (FIG. 1 a, also FIG. 21 a). Conditionally controlled by a LoxP-puro-stop-LoxP cassette, the ubiquitous CAG promoter drives the expression of the FRT-flanked SAM components (aptamere-fused activator domains of p65 and HSF1) separated via a P2A element from dCas9, C-terminally coupled to the transcriptional activator domains VP64, p65 and Rta (VPR) (FIG. 5 a, b; also FIG. 28 a, b). The correct integration of the construct was confirmed via southern blot analysis; animals of the F1 generation showed a normal Mendelian inheritance (FIG. 5 c; also FIG. 28 c). Appropriate astrocytic expression of the conditional system and cleavage of the P2A sequence between the SAM activator and the dCas9-VPR was confirmed by western blot analysis (FIG. 5 a; also FIG. 28 a). For in vivo gene activation, the delivery of target-specific sgRNAs, including stem loops for SAM-aptamere binding, is required. Adeno-associated viruses (AAVs) were used to deliver sgRNAs driven by individual Pol III promoters and a FLExed-GFP marker gene (FIG. 1 a; also FIG. 21 a). In case more sgRNAs are required, two AAVs can be used with a split-FLExed-GFP (FIG. 5 d, e; also FIG. 28 d, e) (Foglieni et al., 2017). The FLEx-system (Cre-ON) is a reporter system based on an inverted and LoxP flanked GFP gene cassette, which is re-inverted and expressed in a Cre-dependent manner, to specifically highlight AAV-infected target cells (Torper et al., 2015). To verify the functionality in vivo, Rosa26-dCas9-activator (dCAM) mice were crossed with an astrocyte-specific Cre (Gfap-Cre) transgenic mouse line, resulting in Cre-specific expression of the activator in astrocytes. Western blot analysis from primary astrocytic lysates confirmed dCas9 expression exclusively in dCAM x Gfap-Cre double positive animals (FIG. 5 c; also FIG. 28 c). Importantly, we confirmed multiplexed endogenous gene activation in primary astrocytes (FIG. 1 b, also FIG. 21 b; FIG. 6 a, b; also FIG. 30 a, b), while ectopic expression of the miRNA-218 (Rivetti di Val Cervo et al., 2017) was evaluated in Neuro2A cells using RT-qPCR (data not shown).

### Example 2: Results: dCAM Based Reprogramming of Astrocytes into Induced Neurons in vivo

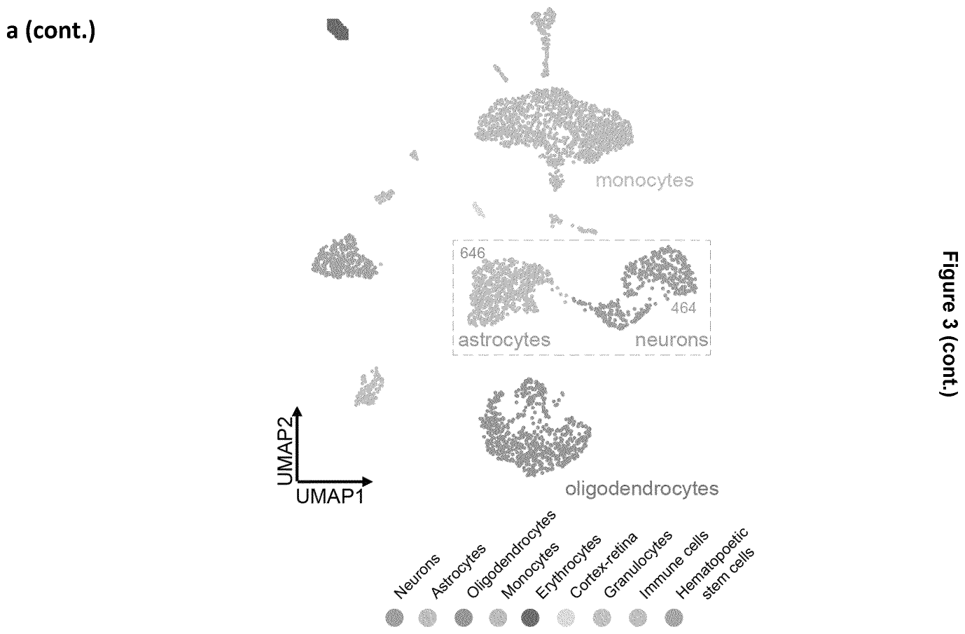

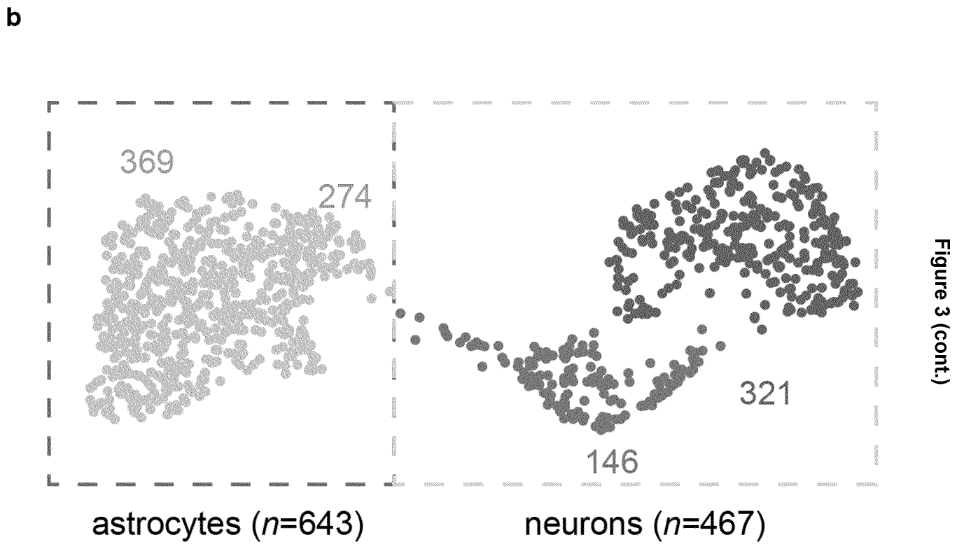

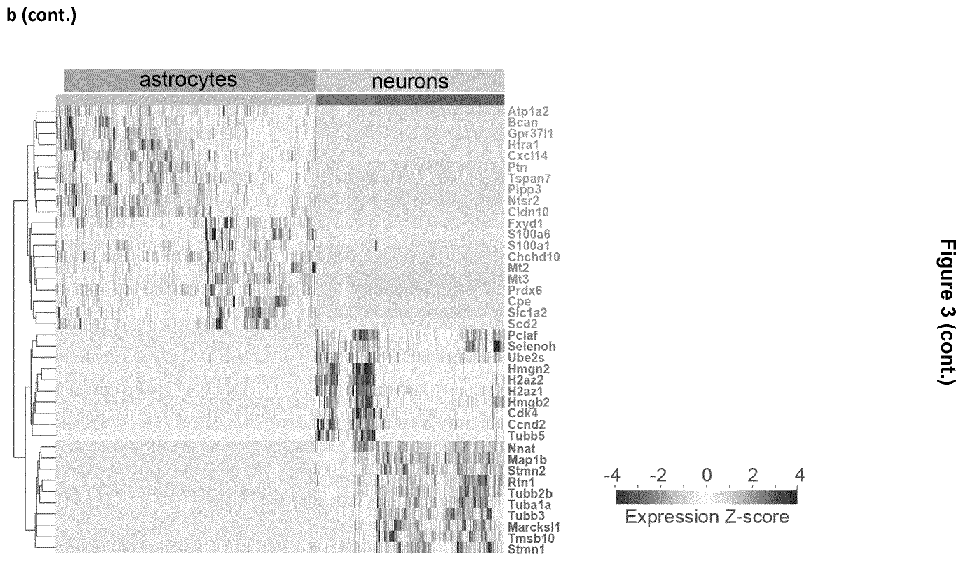

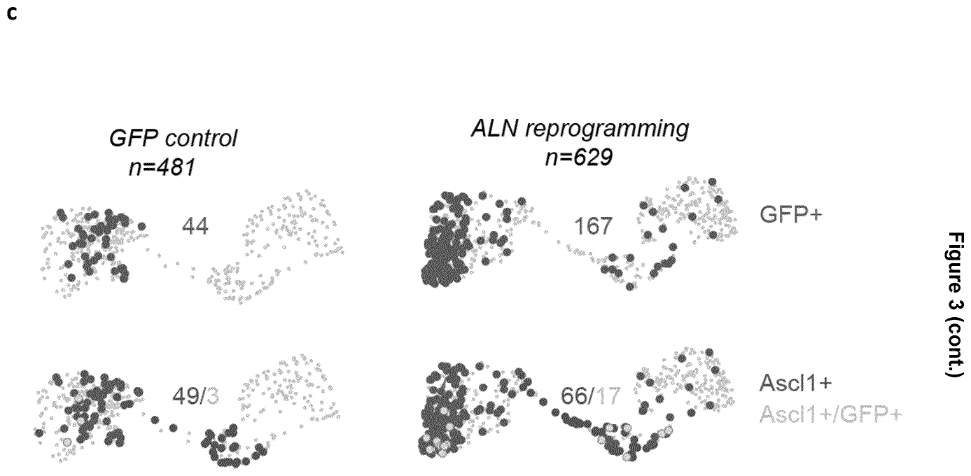

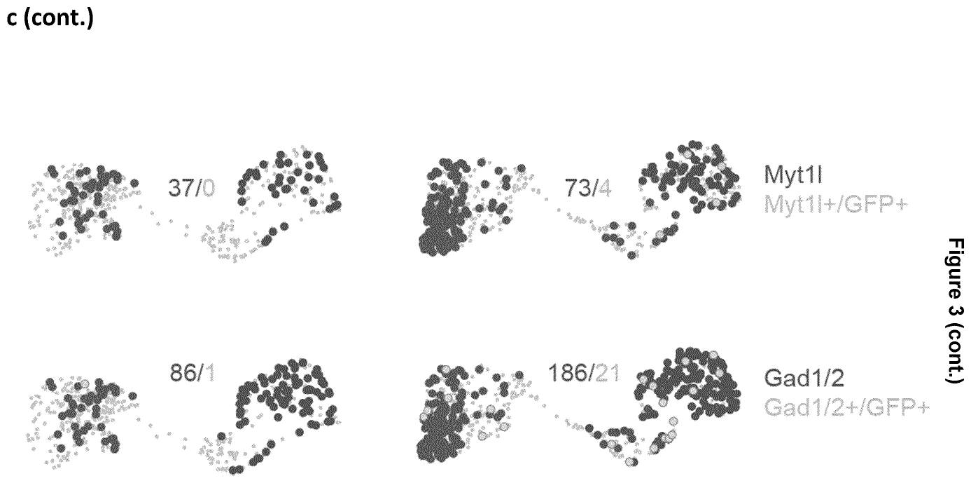

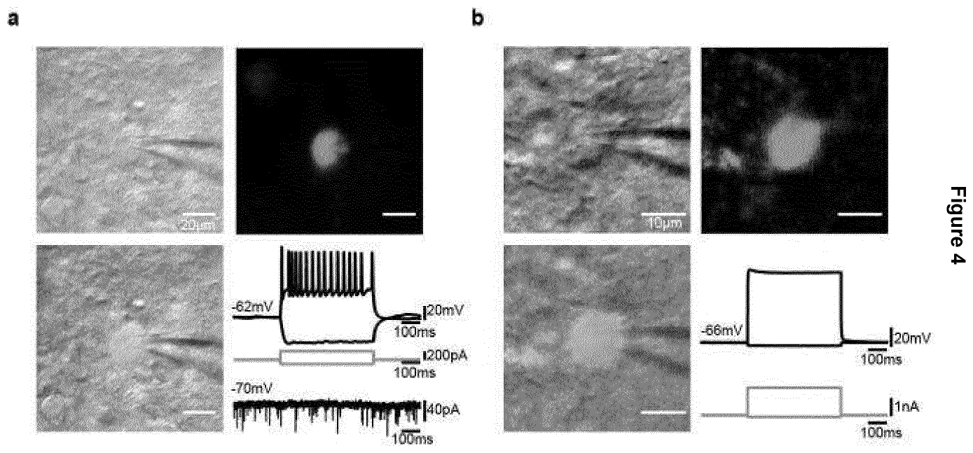

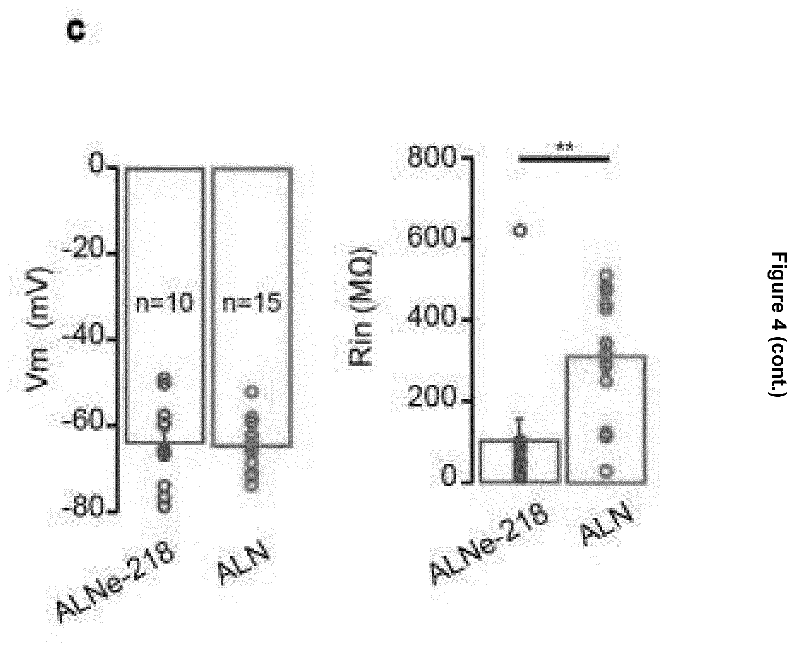

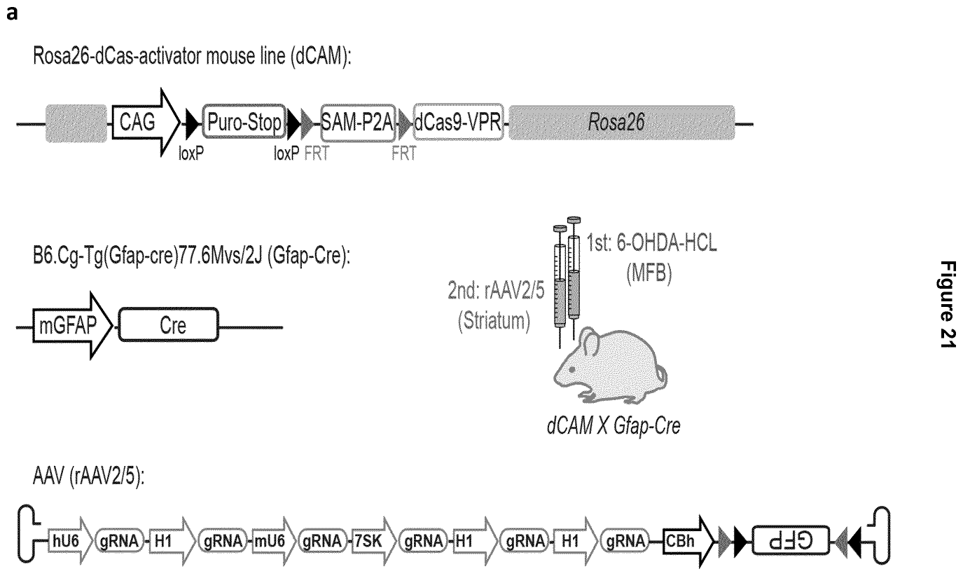

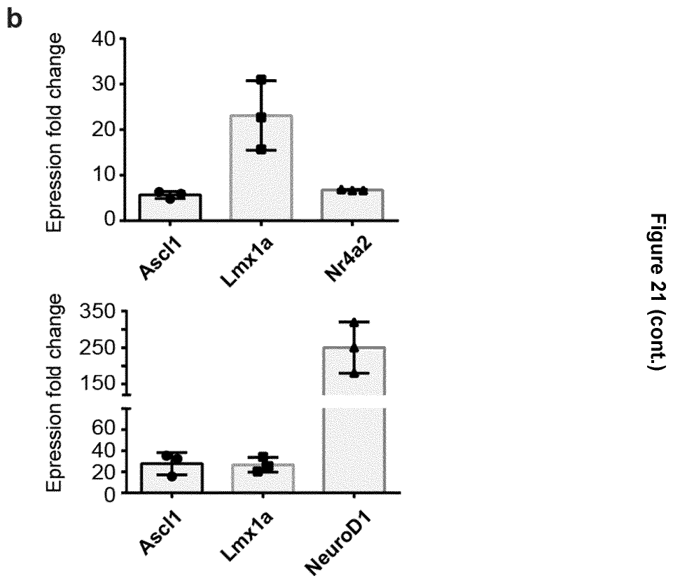

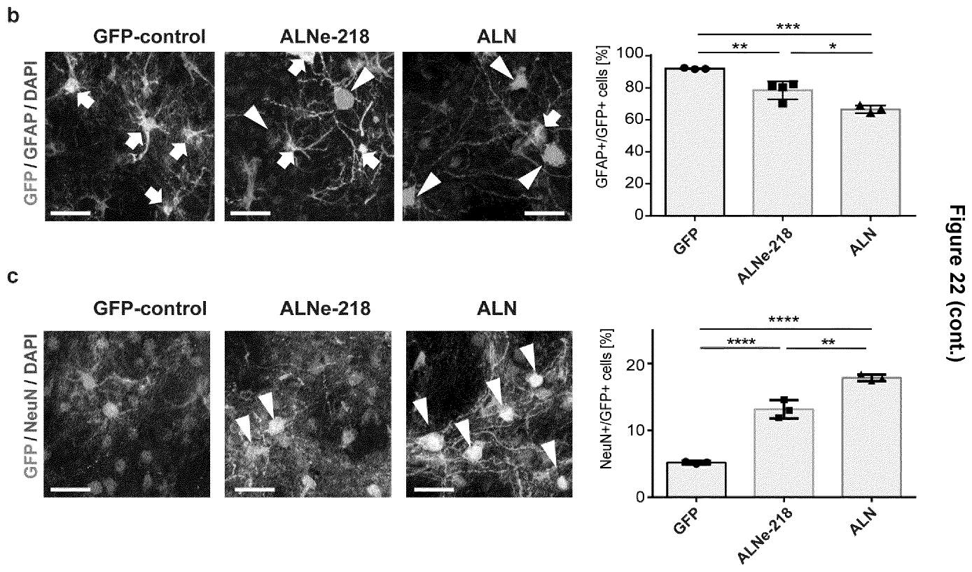

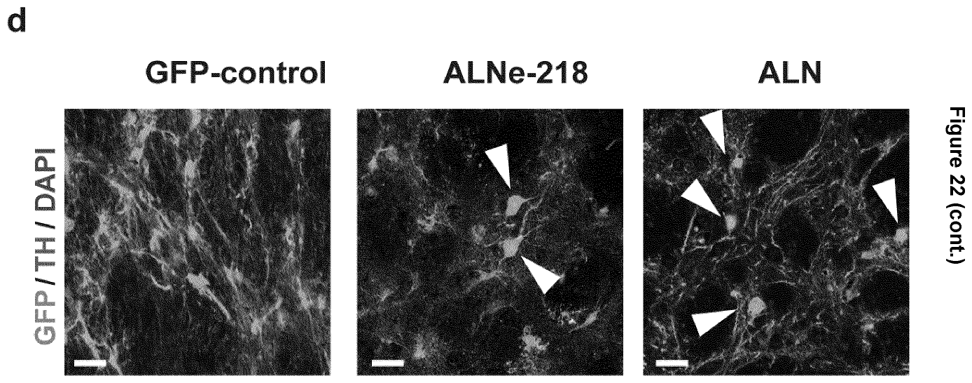

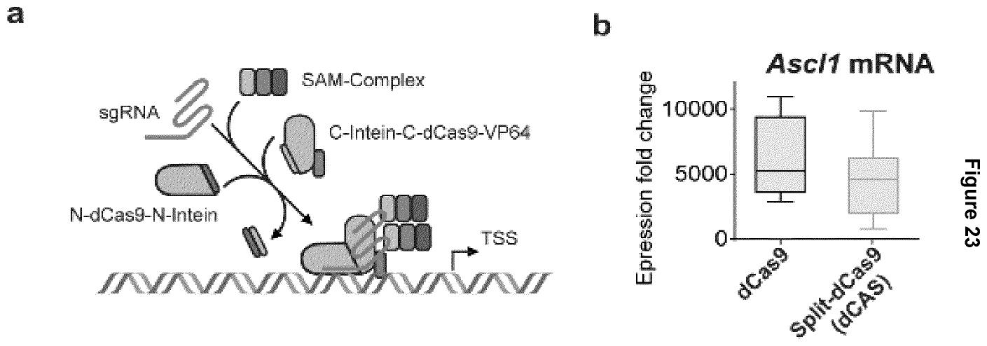

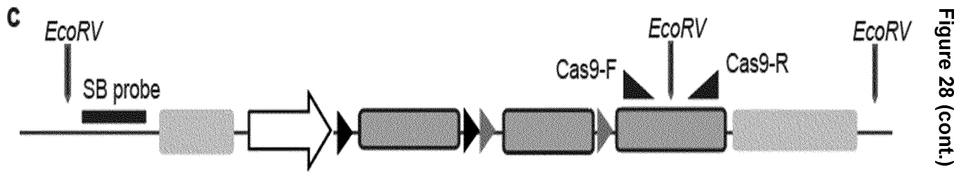

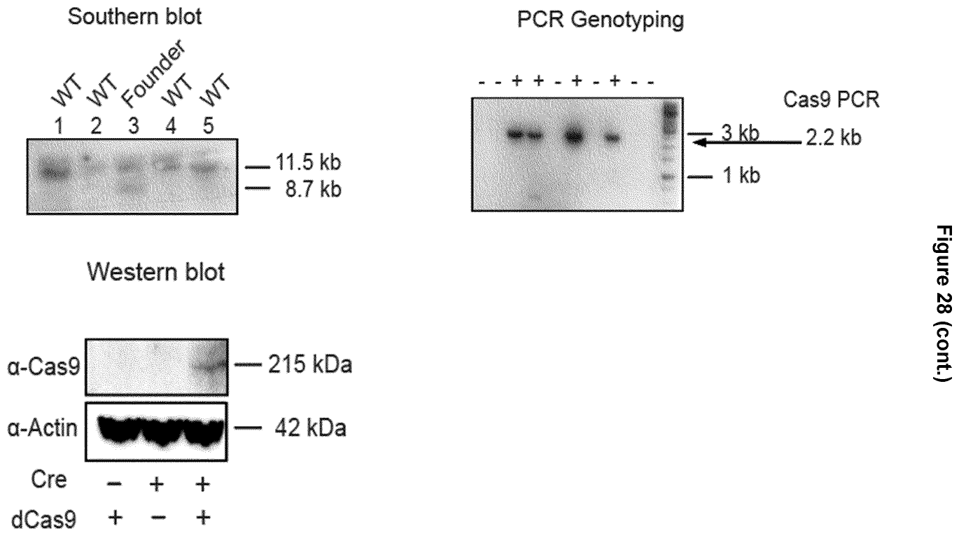

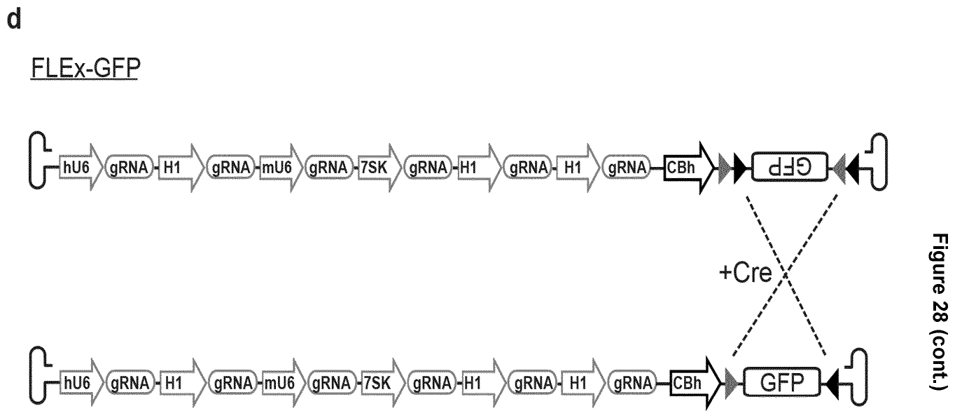

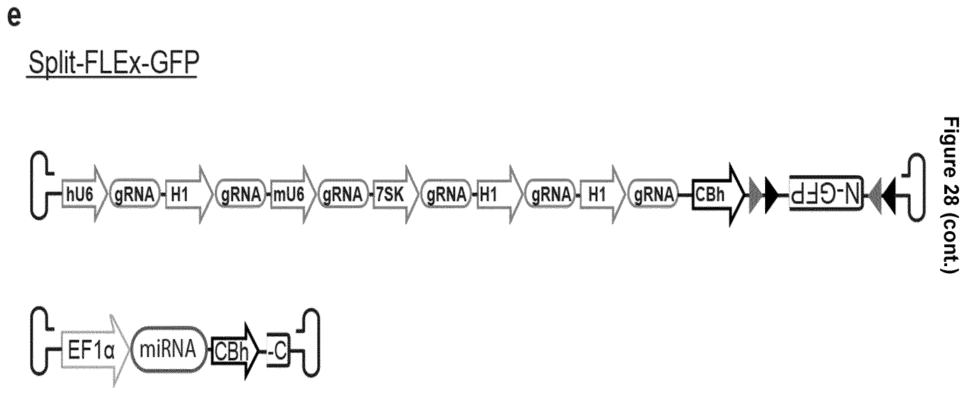

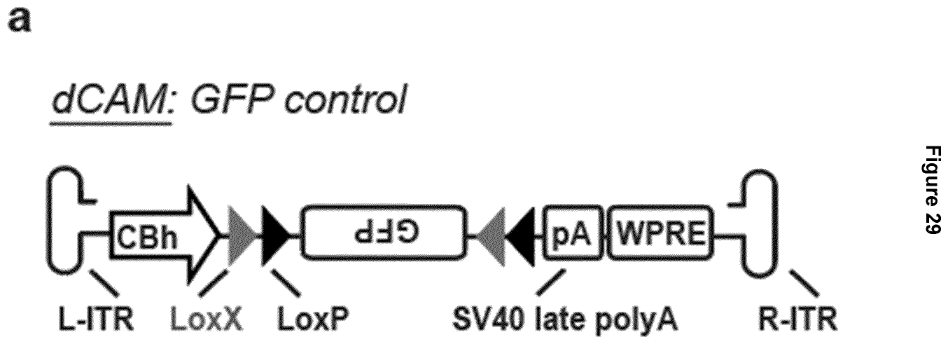

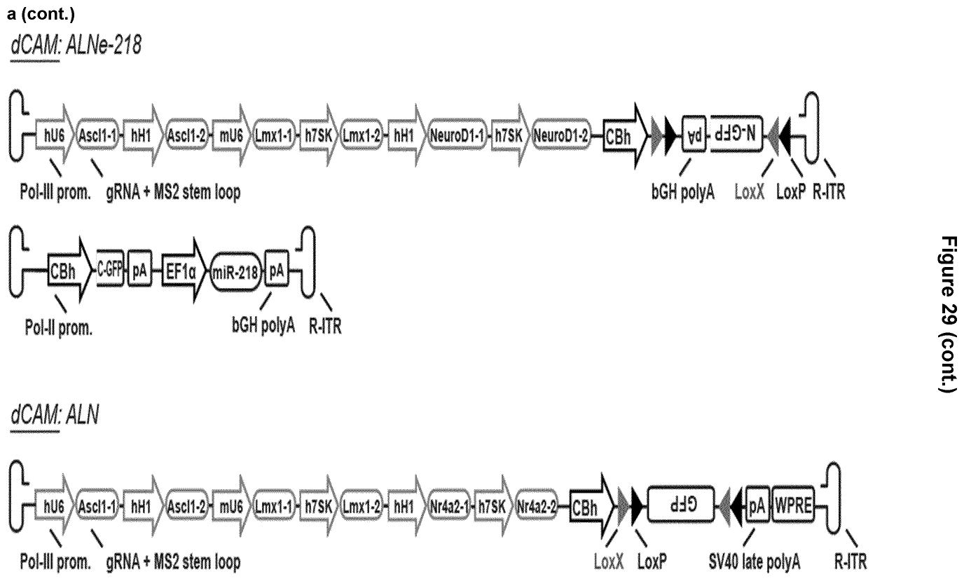

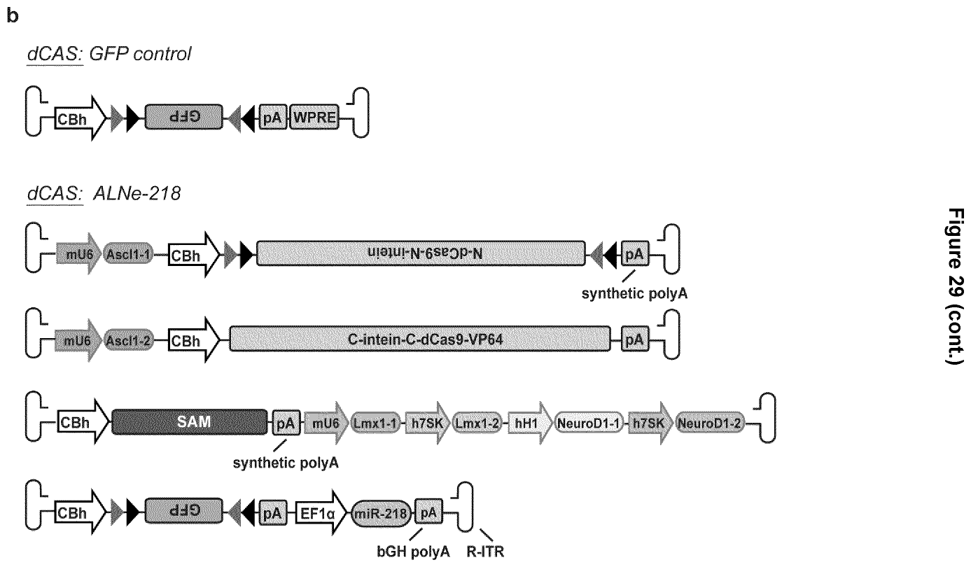

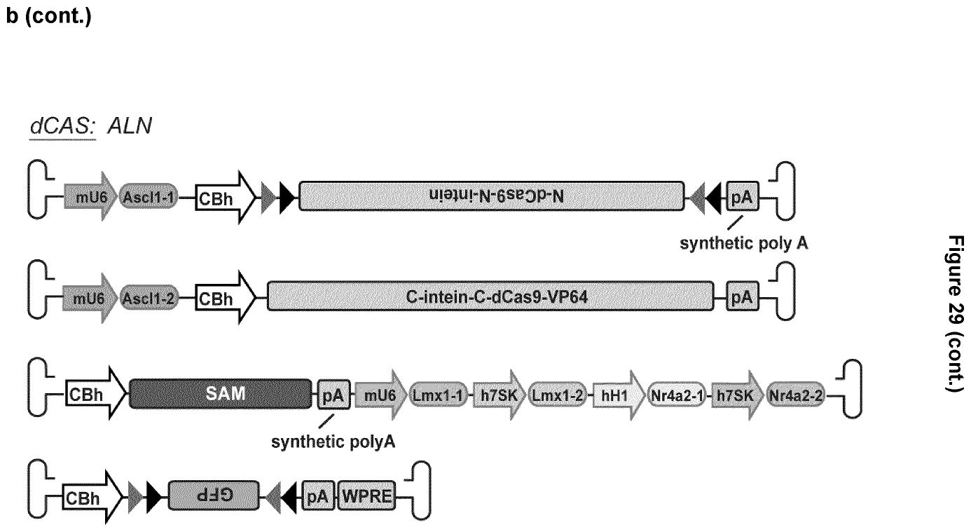

To model advanced stages of PD in mice, we utilized the well-established 6-hydroxydopamine (6-OHDA) toxin model. dCAM x Gfap-Cre double transgenic mice, expressing CRISPRa specifically in astrocytes, were subjected to a unilateral injection of the neurotoxin into the medium forebrain bundle (MFB) at the age of 12-16 weeks, resulting in an efficient and reproducible lesion of the dopaminergic neurons, primarily in the ipsilateral SNpc and their projections into the striatum (Gregorian et al., 2009). This injury promotes reactive gliosis in the striatum, indicated by the upregulation of Gfap (FIG. 7; also FIG. 31) (Grealish et al., 2010; Guo et al., 2014; Schlachetzki et al., 2014). Two weeks after 6-OHDA injection, two sets of sgRNAs, either targeting the promoter regions of the transcription factors Ascl1, Lmx1a, Nr4a2 (ALN) or targeting Ascl1, Lmx1a, NeuroD1 and ectopically expressing miRNA218 (ALNe-218) and mock FLEx-GFP (GFP-control), were delivered via stereotactic injection of 1 μl high titer AAV2/5 into the dorsal striatum. The mice were then comprehensively analyzed 5 and 13 weeks post injection, respectively (FIG. 1c; also FIG. 21 c). We first determined the infection efficiency of astrocytes in our experimental set-up. The different sets of AAVs resulted in a comparable amount of infected GFP+ cells (FIG. 8; also FIG. 24). Checking for the most abundant neurotransmitter systems of the forebrain revealed, that the majority of GFP+ cells were not positive for the glutamatergic marker vGLUT1 but colocalizing for the GABA(gamma-aminobutyric acid)-ergic marker Gad65/67 (FIG. 24d). Quantification revealed that this is the case for almost all induced neurons not only in the AAV-dCAS but also in the dCAM setting (dCAM: GAD65/67+/GFP+91.93±6.53%; AAV-dCAS: GAD65/67+/GFP+ 93.60±5.35%) (FIG. 24e). IHC analysis of mice, injected two weeks after the 6-OHDA lesion with FLEx-GFP control virus, revealed that 5 wpi 97.13±0.45% of GFP positive cells were astrocytes, as they expressed the astrocytic marker glial fibrillary acidic protein (Gfap) (FIG. 9 b; also FIG. 33). Immunohistochemical stainings of GFP have been performed in animals 13 week after viral injection. The initial analysis of transduction efficiency showed comparable amounts of GFP+cells between the experimental groups (FIG. 32 b). Accordingly to previous reports using FLEx-GFP in combination with this Cre-line (Mattugini et al., 2019), about 4% (3.9±0.53%) were positive for the neuronal marker NeuN (RBFOX3, FIG. 9 d; also FIG. 33). Next, we determined the efficiency of reprogramming achieved by different sgRNA combinations. At five weeks post AAV injection both combinations (ALN and ALNe-218) still showed a high proportion of GFAP+/GFP+ double positive cells (FIG. 9 a, b; also FIG. 33), with an increased percentage of NeuN+/GFP+ cells to 14.77±3.09% for ALN and 15.67±0.96% for ALNe-218 (FIG. 9 c, d; also FIG. 33). After additional 8 weeks (13 wpi), the proportion of GFAP+/GFP+ cells significantly decreased to 66.57±2.35% for ALN and 78.45±5.63% for ALNe-218 (FIG. 1 d, e; also FIG. 21). Conversely, the proportion of NeuN+neurons amongst GFP+transduced cells further increased to 17.87±0.50% in striata treated with the ALN-inducing sgRNA combination. Interestingly, such marked increase was not observed for the ALNe-218 sgRNAs (NeuN+/GFP+ 13.17±1.36%) (FIG. 1 f, g; also FIG. 22). Thus, the ALN combination is more efficient to induce neuronal conversion of striatal astrocytes after 6-OHDA lesion.

### Example 3: Results: AAV Based Split-dCas9-Activator System (AAV-dCAS) for Endogenous Gene Activation

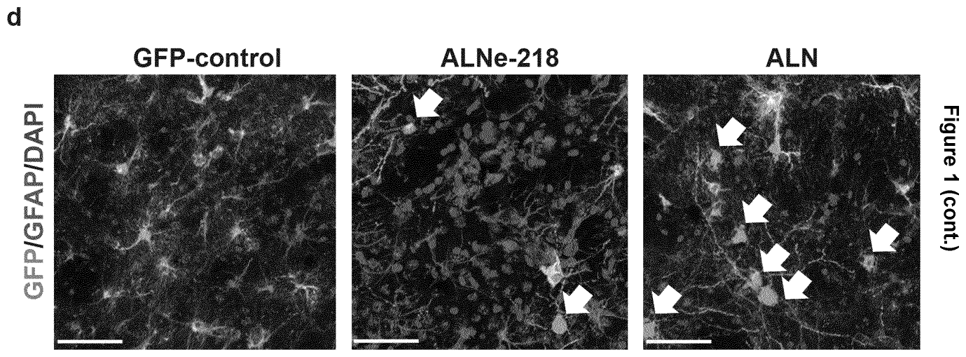

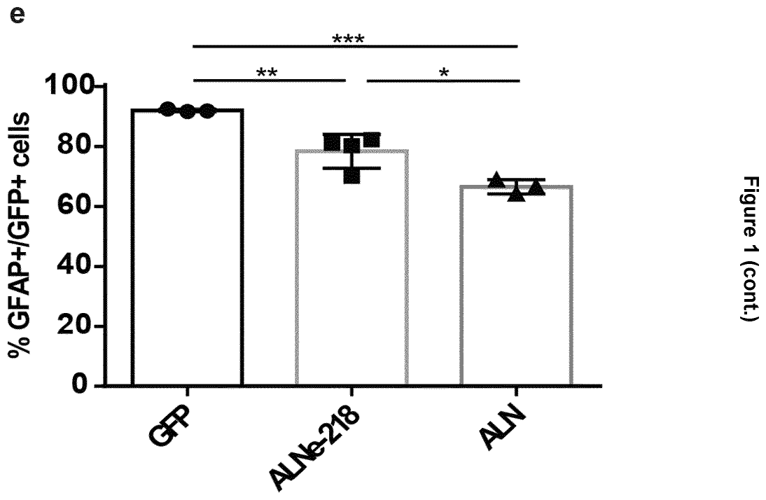

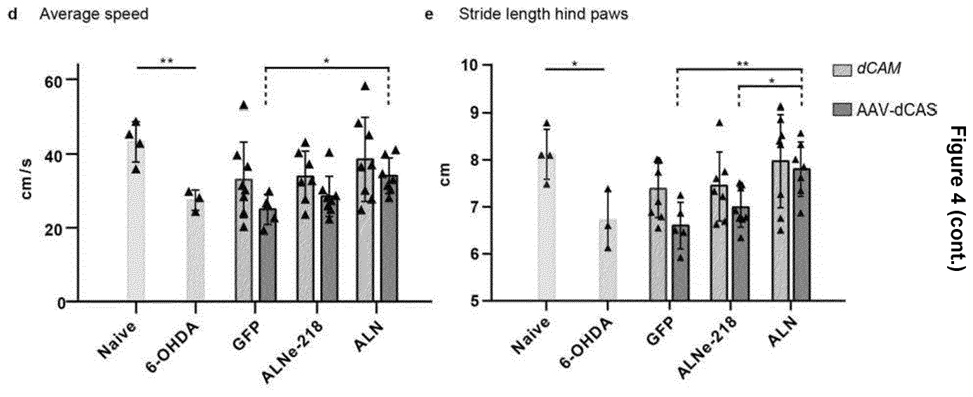

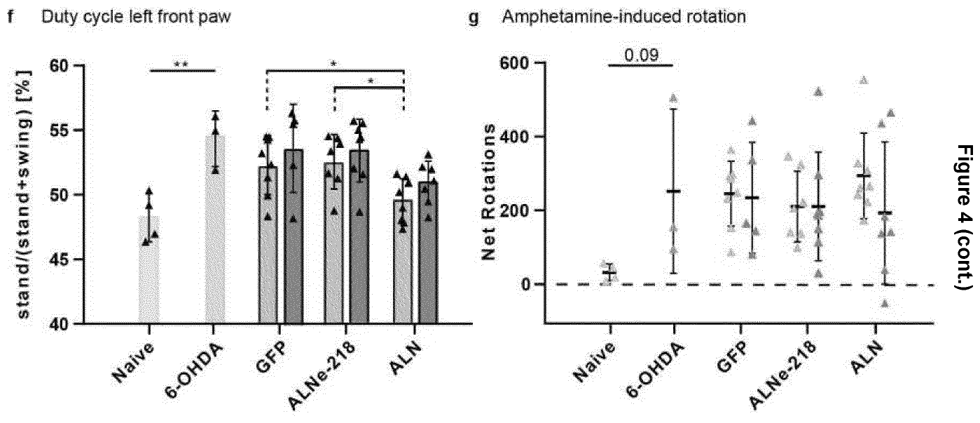

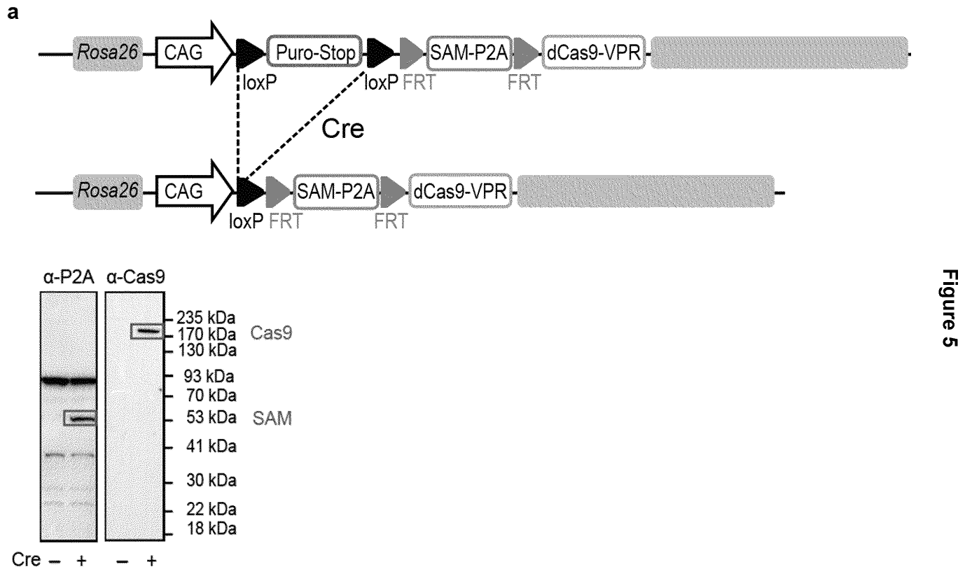

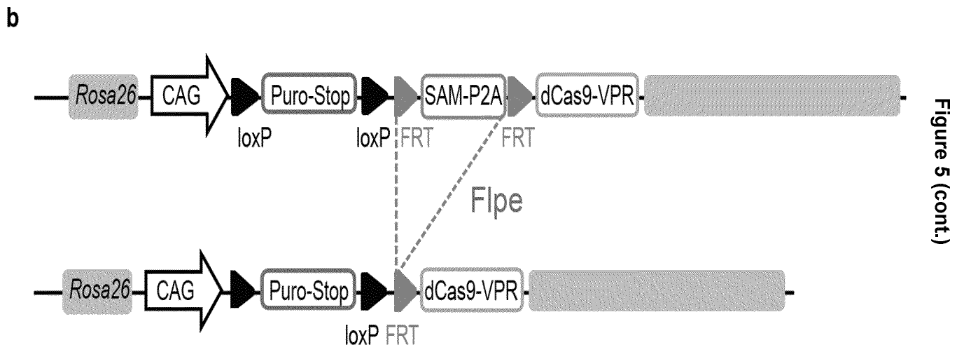

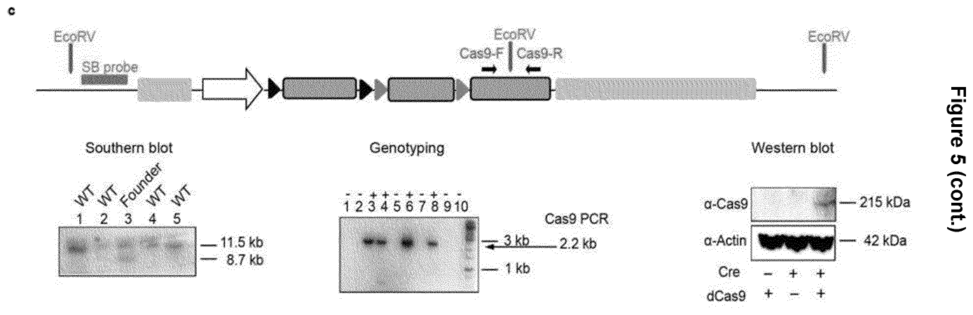

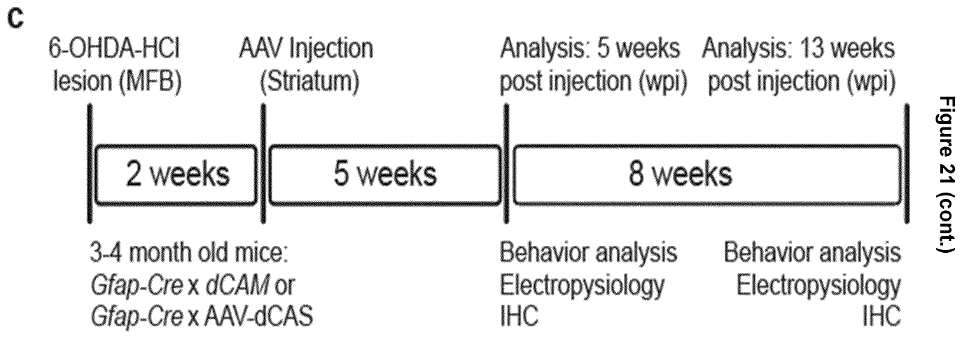

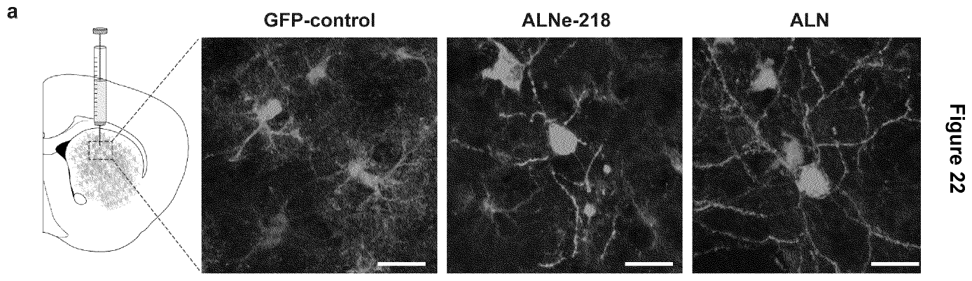

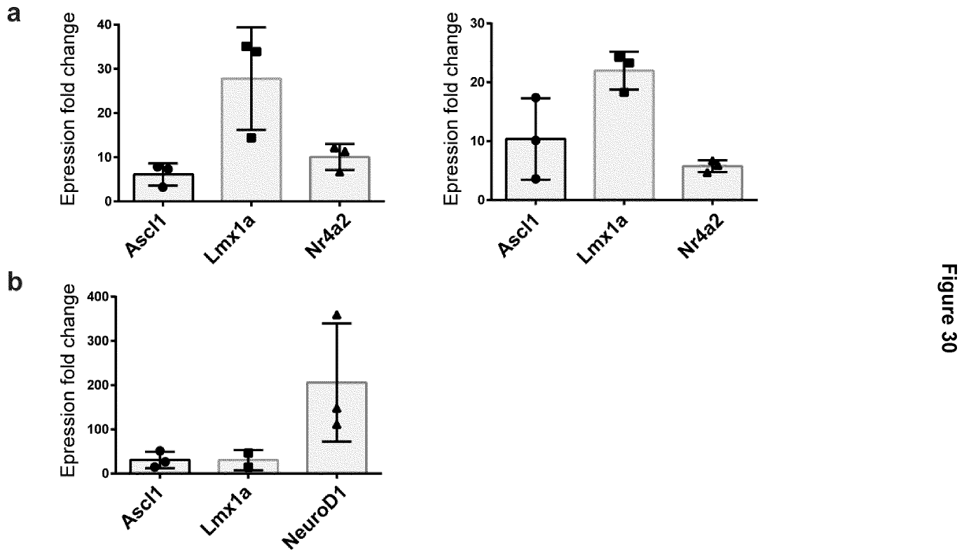

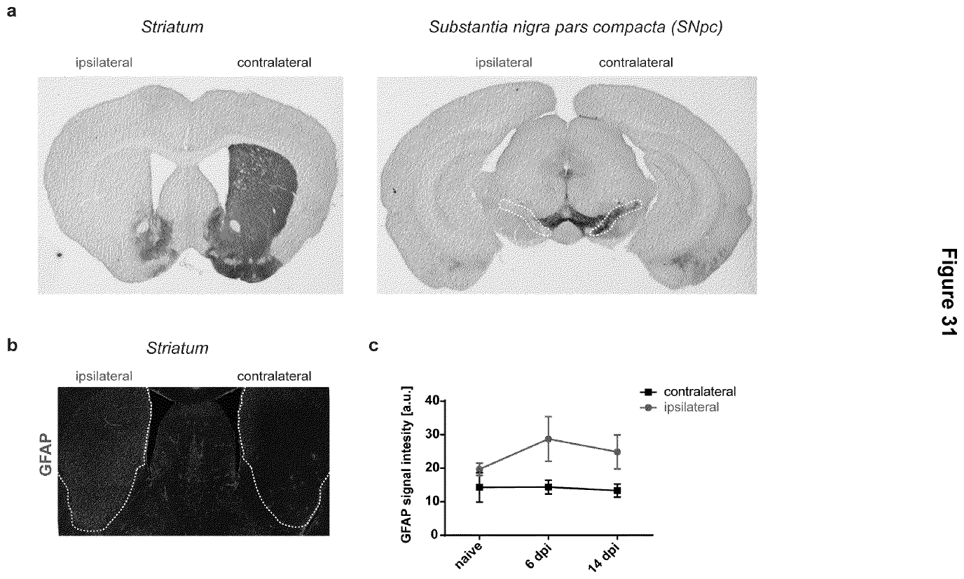

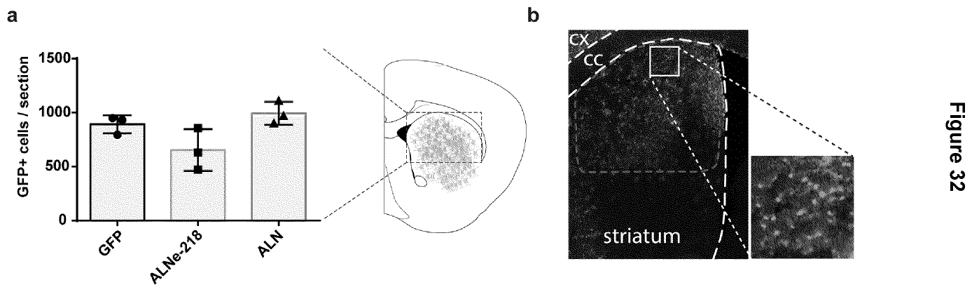

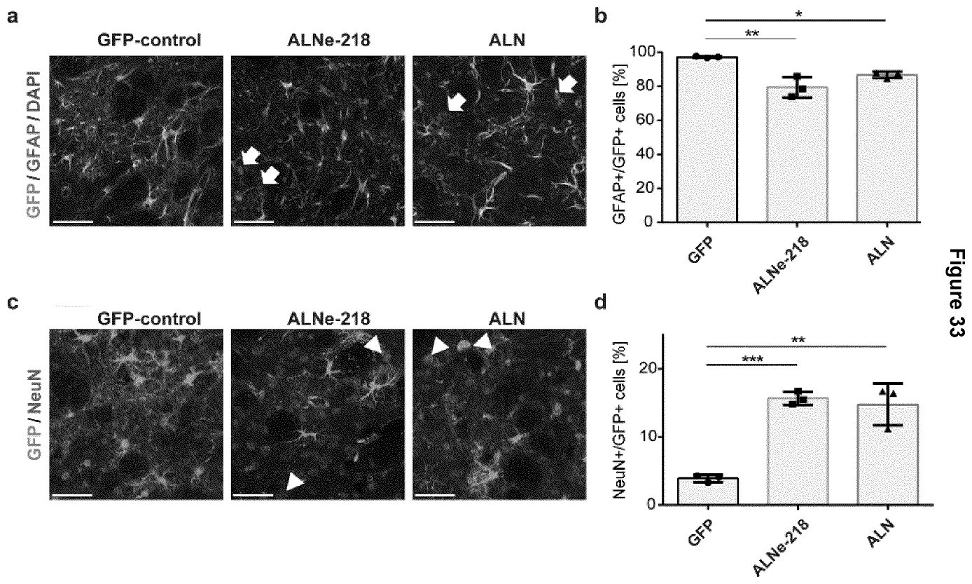

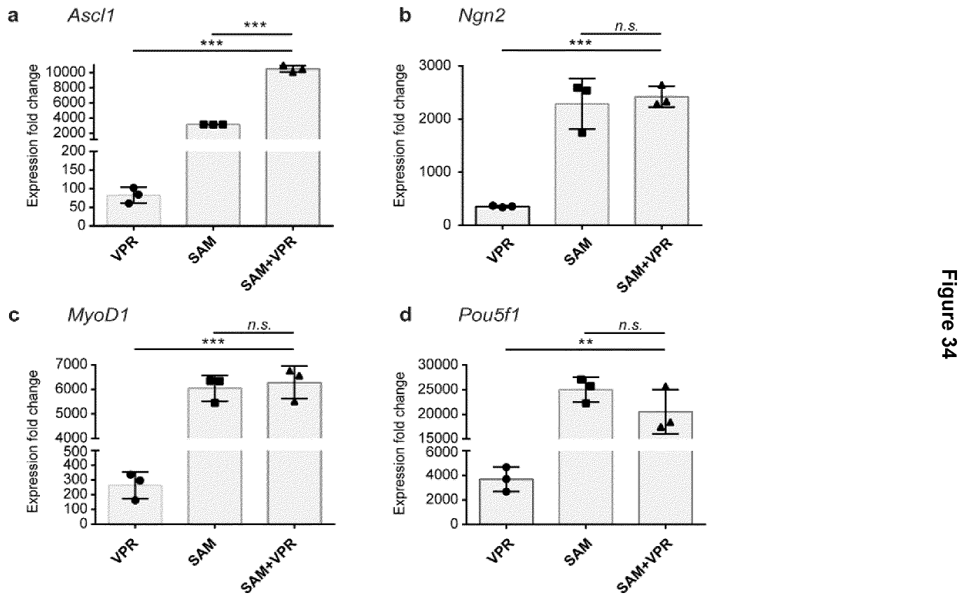

The efficient and thorough reprogramming via CRISPRa gene activation observed in our dCAM line encouraged us to generate a universal tool, independent of transgenic recipients, by delivering the complete CRISPRa system via AAVs. It is important to render these reprogramming approaches versatile for a broad range of applications, such as usage in other model organisms like non-human primates, and ultimately, for cell reprogramming therapies in patients. Gold standard for the delivery of the sgRNAs are AAVs, as they exhibit low immunogenicity and ensure high and sustained expression (Grieger and Samulski, 2005; Mattugini et al., 2019; Zaiss and Muruve, 2005). To circumvent the low packaging capacity of AAVs, we applied a split-intein approach to the dCas9-SAM system suitable for AAV (AAV-dCAS) integration. As our in vitro studies have shown that the SAM activator system alone is sufficient to provide robust gene induction (FIG. 10; also FIG. 34), we generated a split version of the fusion protein dCas9-VP64 (4× VP16 activator domain, herpes simplex virus protein vmw65) into two parts: each part was fused to corresponding split-intein moieties (AAV-N-dCas9aa1-573-N-intein and AAV-C-dCas9aa574-1368-VP64-C-intein). Thus, upon the co-expression of these two AAV constructs, intein-mediated trans-splicing leads to the reconstitution of full-length dCas9-VP64 protein (FIG. 2 a; also FIG. 23 a) (Truong et al., 2015). The remaining elements of the SAM system were packed onto an independent AAV vector together with four sgRNAs driven by heterologous Pol III promoters (FIG. 2 d; also FIG. 23 d). The successful reconstitution into full-length dCas9-VP64 was confirmed by western blot analysis (FIG. 11 a, b; also FIG. 35). We measured comparable transcriptional activation efficiencies between full-length and split-version of dCas9 when targeting endogenous expression of Ascl1 in Neuro2A cells (FIG. 2 b; also FIG. 23). In primary astrocytic cultures, astrocytes could be reprogrammed efficiently into MAP2+ neurons by the activation of endogenous Ascl1 (FIG. 2 c; also FIG. 23). Multiplexed gene activation was assessed as well. Indeed, up to five endogenous genes were targeted in parallel and showed robust activation on RNA and protein level (FIG. 2 e; also FIG. 23; FIG. 11 c, d; also FIG. 35).

### Example 4: Results: AAV-dCAS Based Reprogramming of Astrocytes into Induced Neurons in vivo

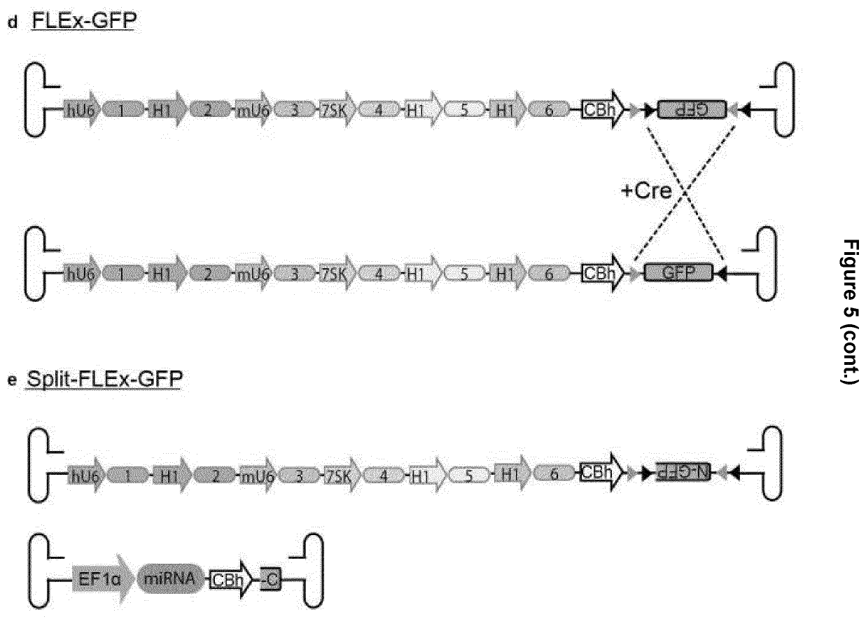

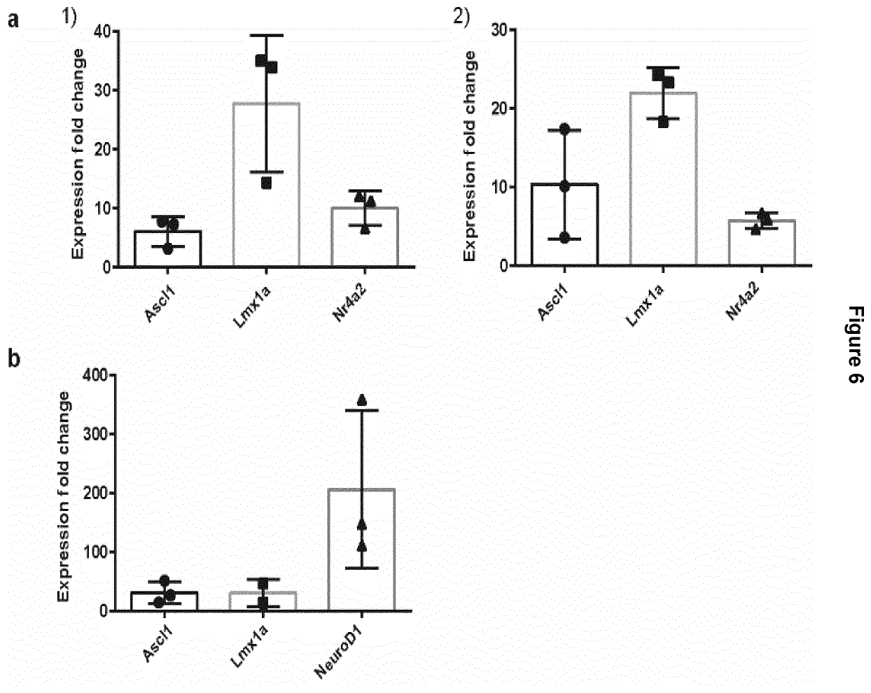

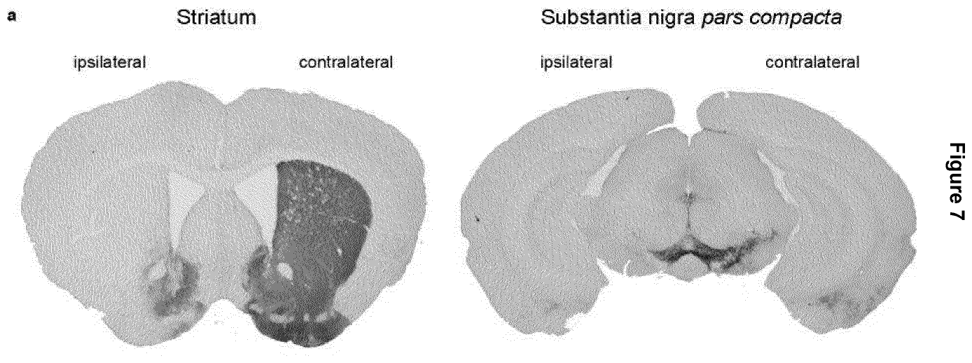

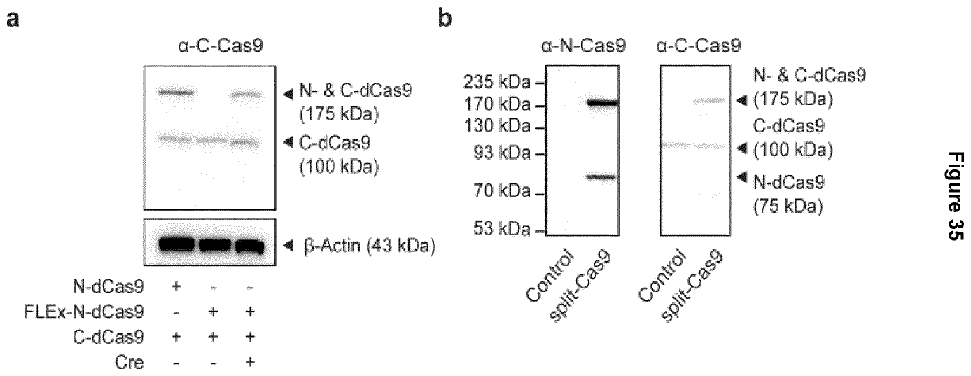

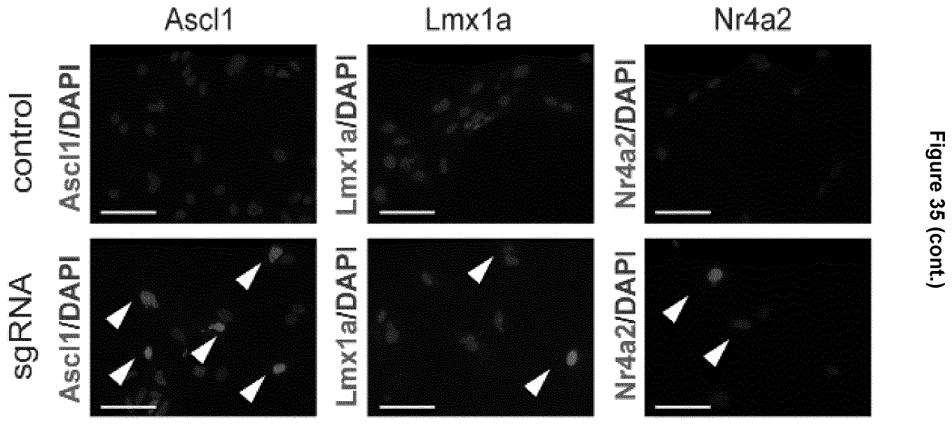

Similar to the dCAM-based reprogramming experiment, we employed the transgenic Gfap-Cre mouse line to ensure astrocyte-specific expression of the reprogramming tools. Experimental setup and timeframe were identical to the dCAM setting; to deliver split-dCas9, a FLEx-N-dCas9 and a C-dCas9-VP64 AAV were used, inducing expression of N-dCas9 in astrocytes (Gregorian et al., 2009; Torper et al., 2015); identical sgRNAs and the FLEx-GFP reporter were delivered by additional AAVs. At 5 wpi, the proportion of different infected cell types was comparable to the results in the dCAM system (FIG. 12 a, b; also FIG. 36). Interestingly, less than 5% (4.23±1.55%) of the of GFP+cells were also NeuN positive in GFP control injected mice, while such percentage increased in both reprogramming conditions, ALN (14.67±1.21%) and ALNe-218 (14.10±0.89%) to about 14% (FIG. 12 c, d; also FIG. 36). At the later time point (13 wpi) the percentage of GFAP+/GFP+ cells decrease to 48.0±6.65% for ALN and 76.23±3.27% for ALNe-218 (FIG. 2 f, g; also FIG. 23), whereas the proportion of NeuN+/GFP+reprogrammed cells increased to 25.47±6.85% upon ALN activation, but not under the ALNe-218 condition (11.67±0.35%) (FIG. 2 h, I; also FIG. 23). Thus, the AAV-dCAS recapitulates the results obtained with the dCAM model, highlighting a higher efficiency of the ALN combination over time. None of the treated animals developed any tumors during the period of the experiment.

### Example 5: Results: Characterization of Cell Identity of Induced Neurons

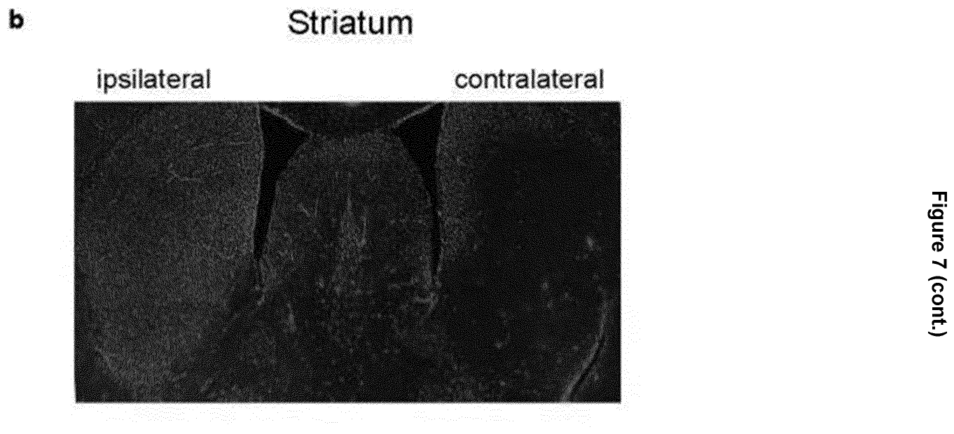

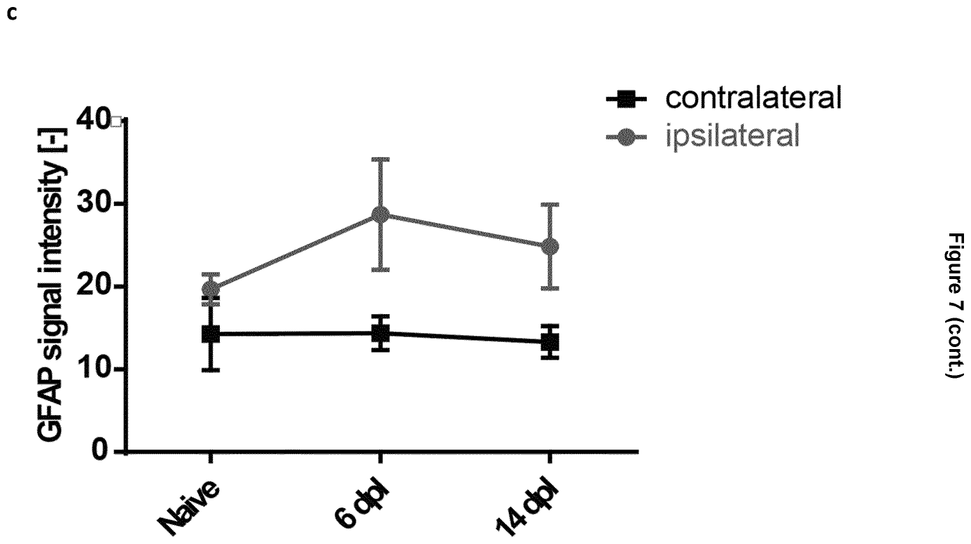

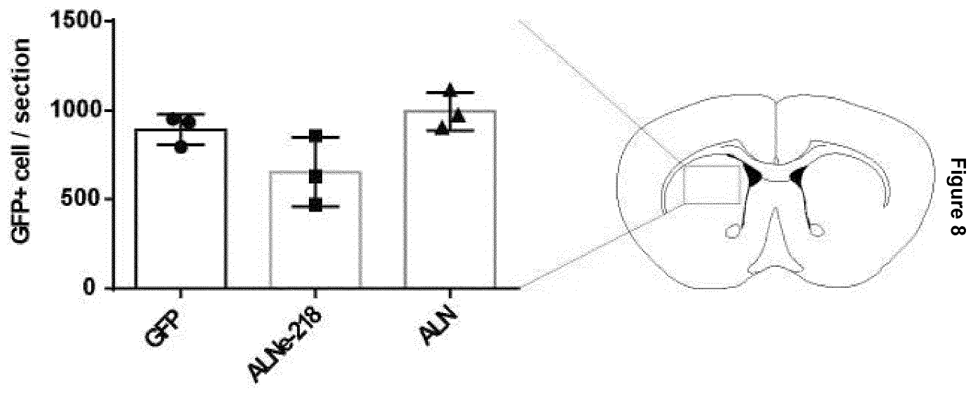

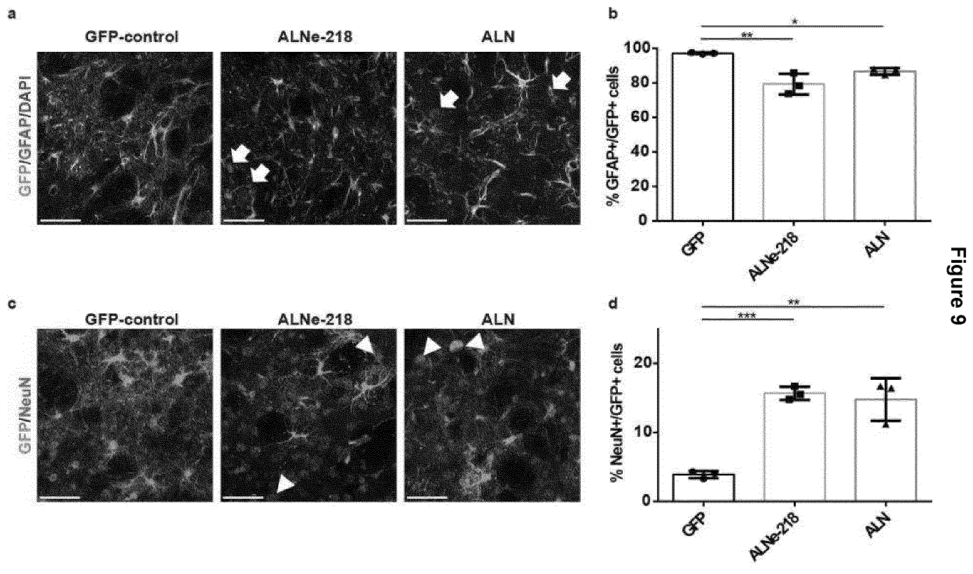

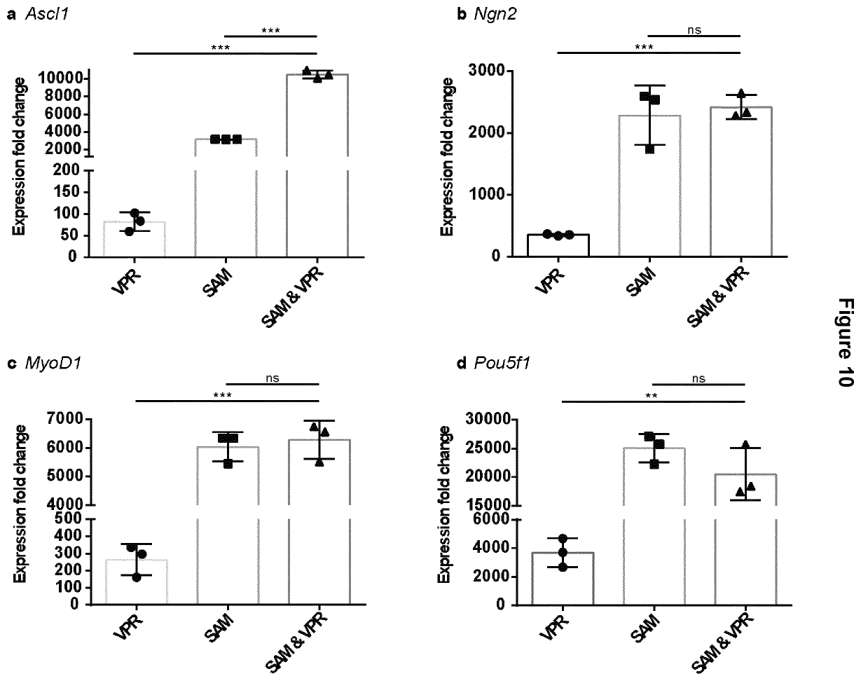

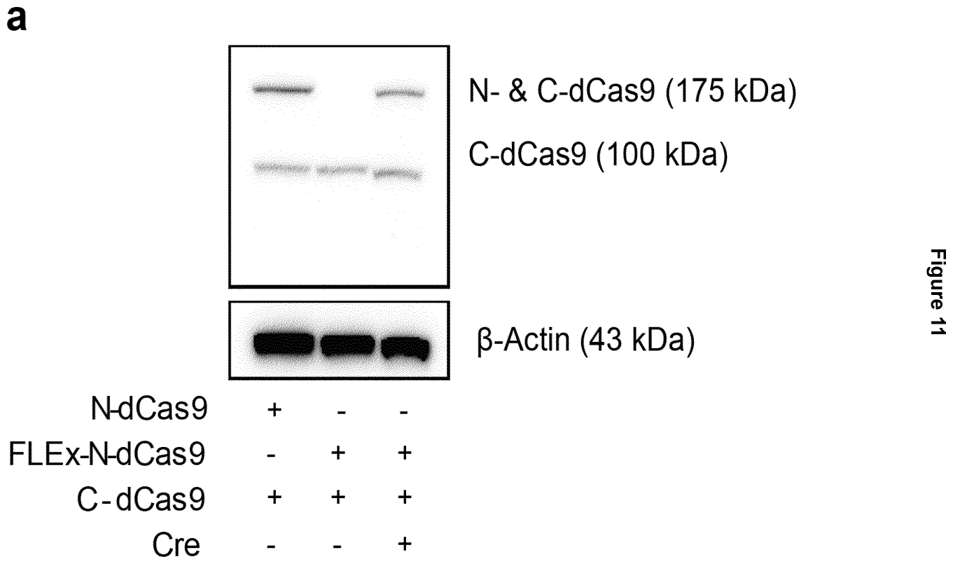

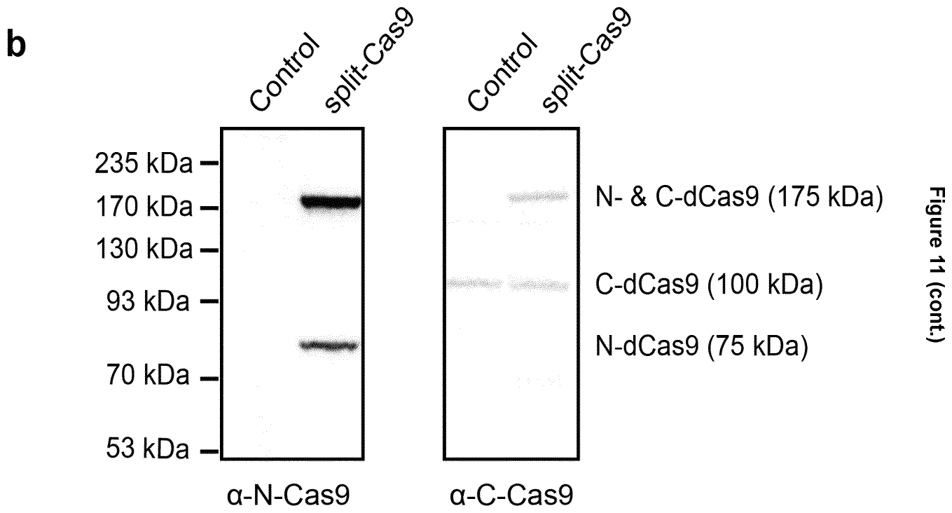

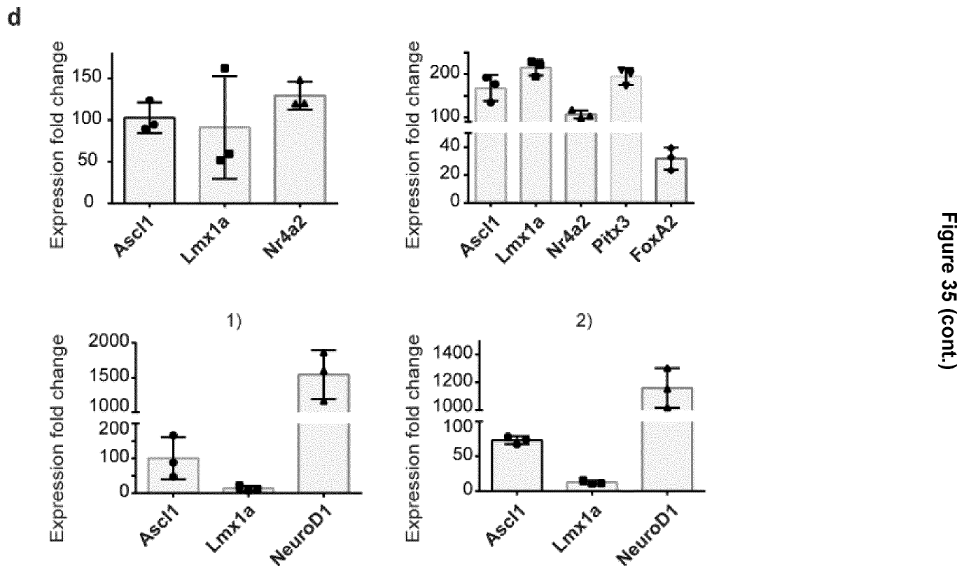

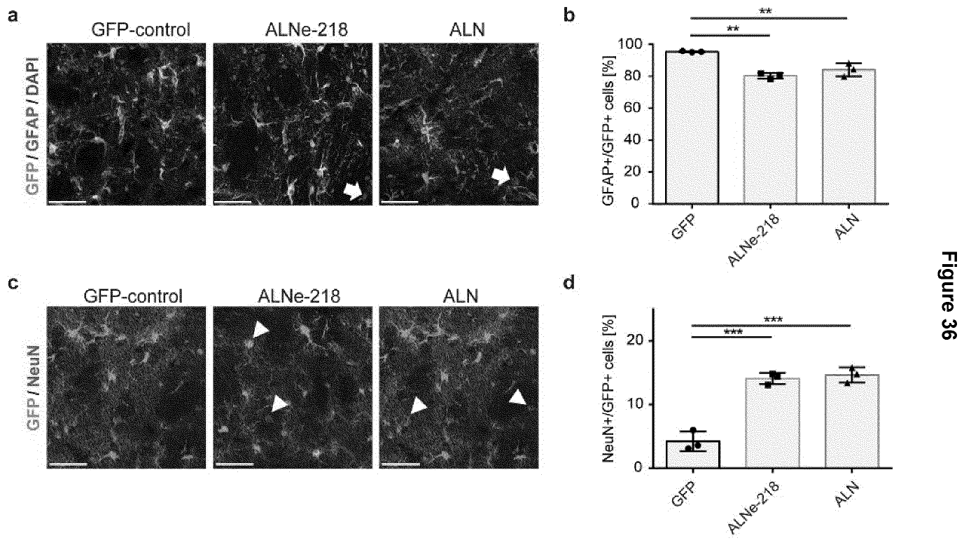

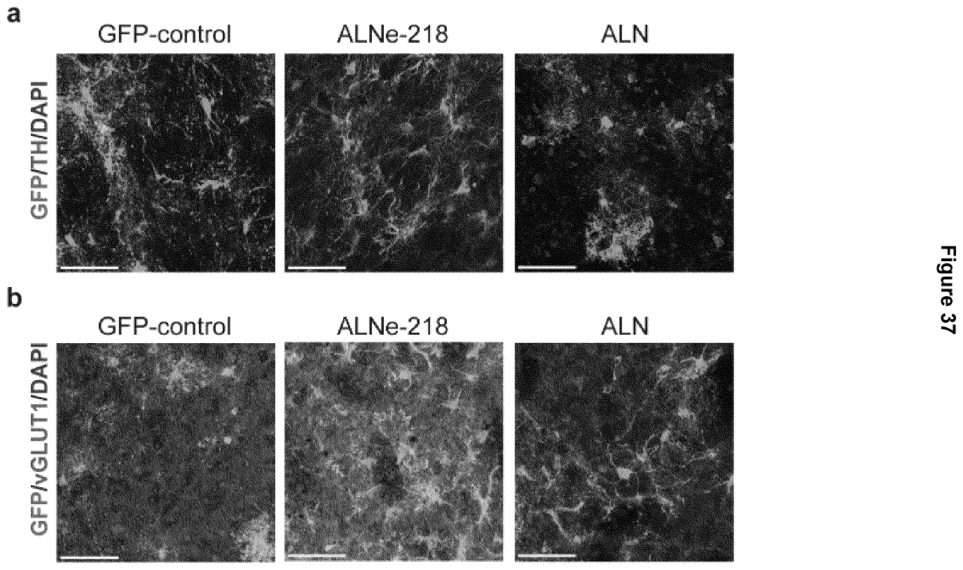

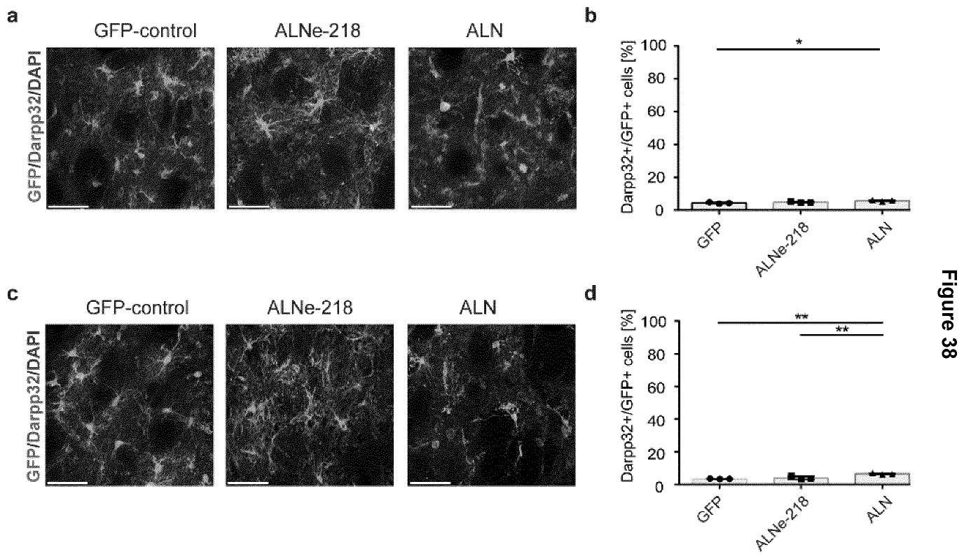

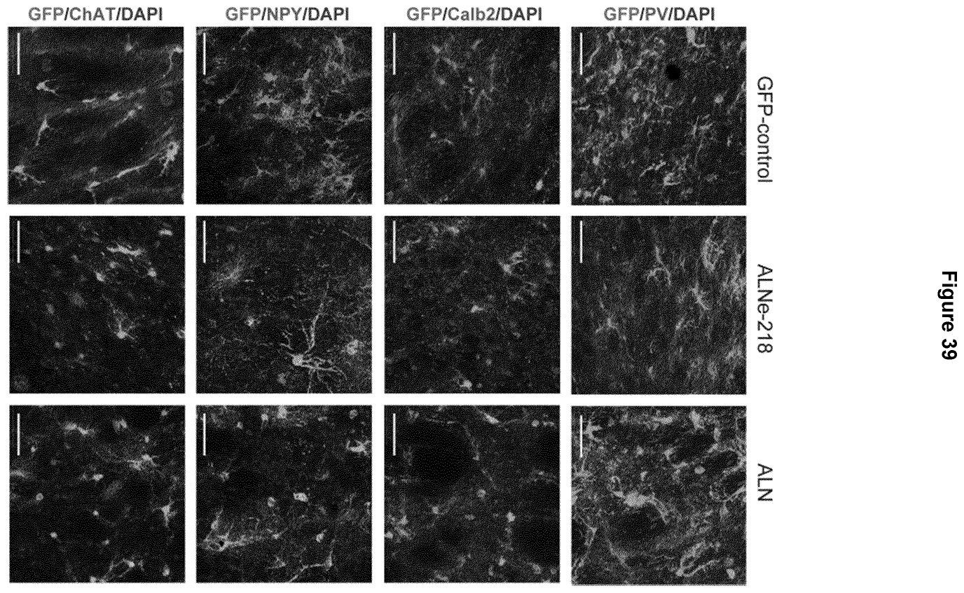

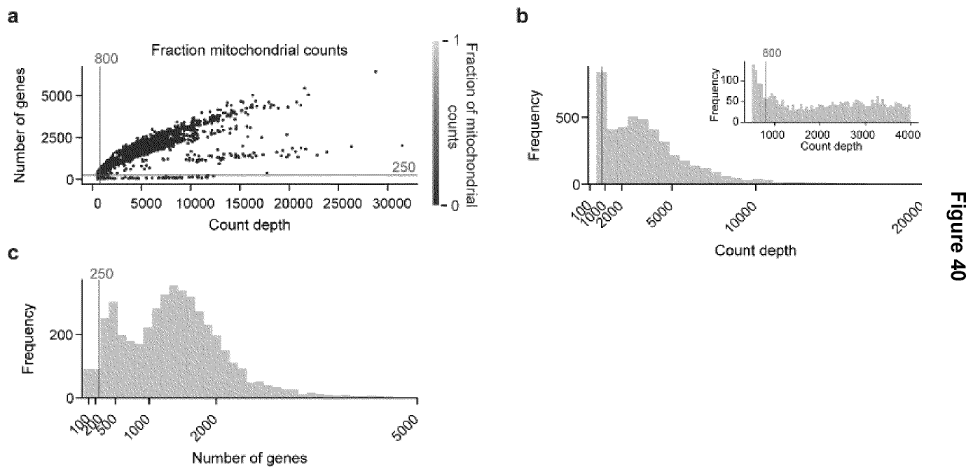

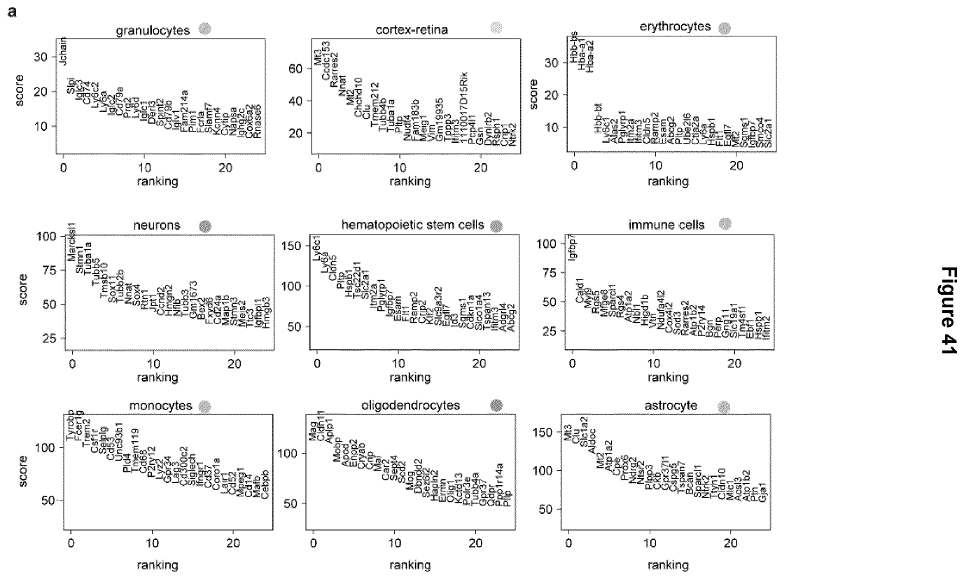

To define the subtype of the induced neurons, immunohistochemical (IHC) analysis on striatal sections of mice 13 wpi was performed. Data is shown for the AAV-dCAS system, and the analysis of dCAM revealed similar results (data not shown). Cells were positive for Gad65/67 a marker for GABAergic neurons (FIG. 2 j; also FIG. 23). In addition, the expression of cell type specific neurotransmitters was evaluated, including tyrosine hydroxylase (TH) for dopaminergic neurons or noradrenergic and vGLUT1 for glutamatergic neurons (FIG. 13; aslo FIG. 37). We could not find any converted neurons expressing TH, even though some TH+ neurons could be detected in the striatum, which, however we also observed in the 6-OHDA treated control animals. Also, cells were not positive for the glutamatergic marker vGLUT1. Taken together, most in vivo induced neurons were not positive for the broad striatal marker DARPP32 (dCAM: ALNe-218 4.7%, ALN 5.7%; dCAS: ALNe-218 4%, ALN 6.4%) nor for interneuron markers like parvalbumin, neuropeptide Y, calretinin and CHAT (FIGS. 14, 15; also FIGS. 38-39).

### Example 6: Results: Single cell RNA-seq Analysis Reveals the GABAergic Fate of Induced Neurons in dCAM Model

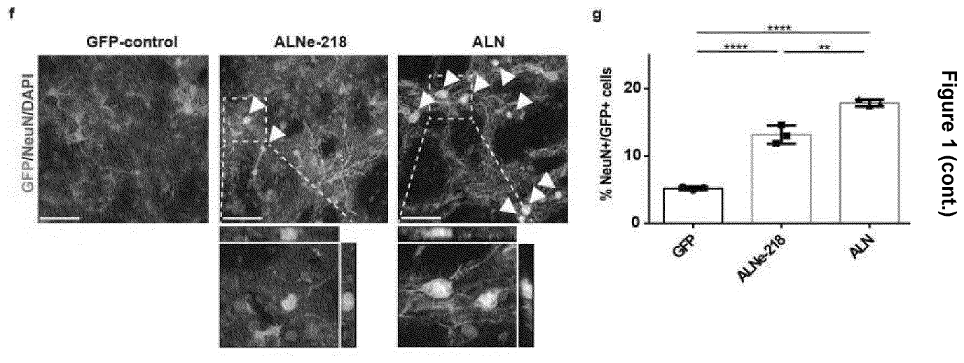

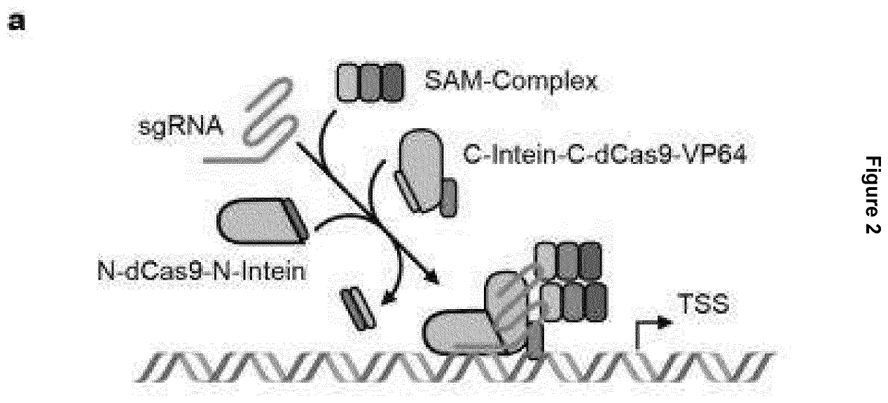

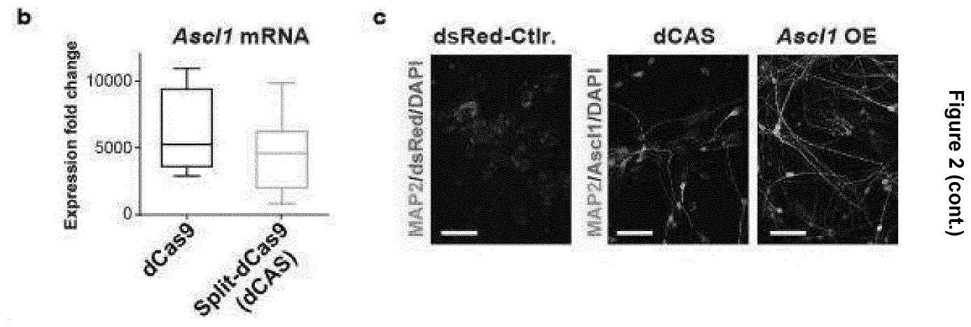

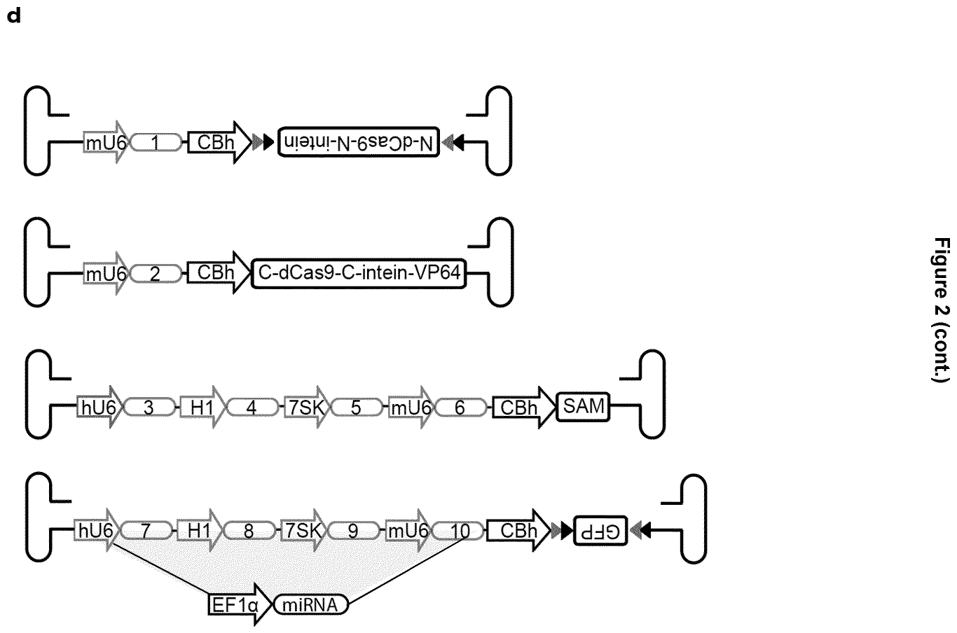

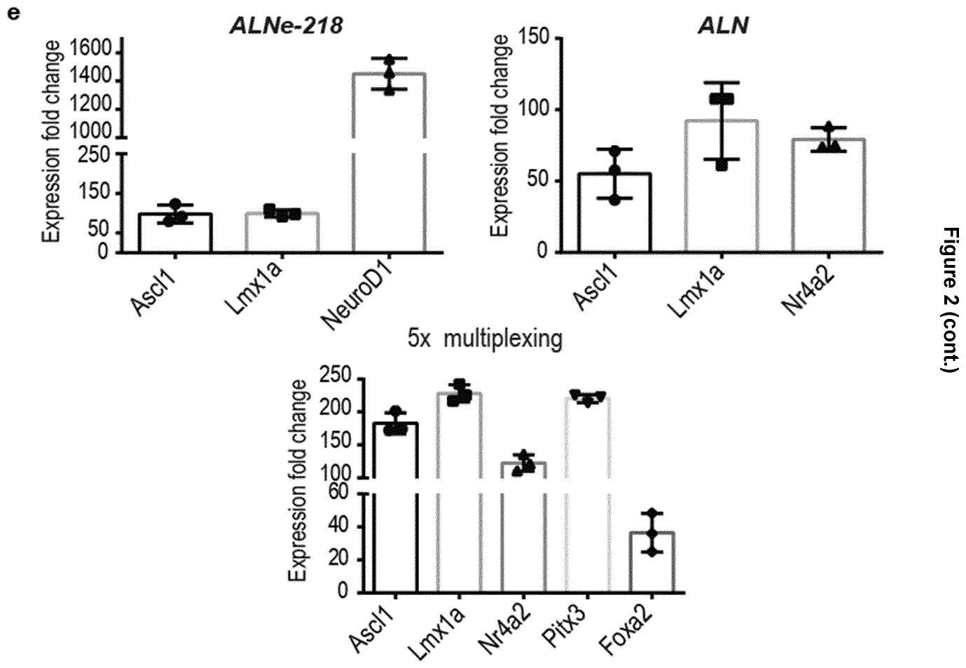

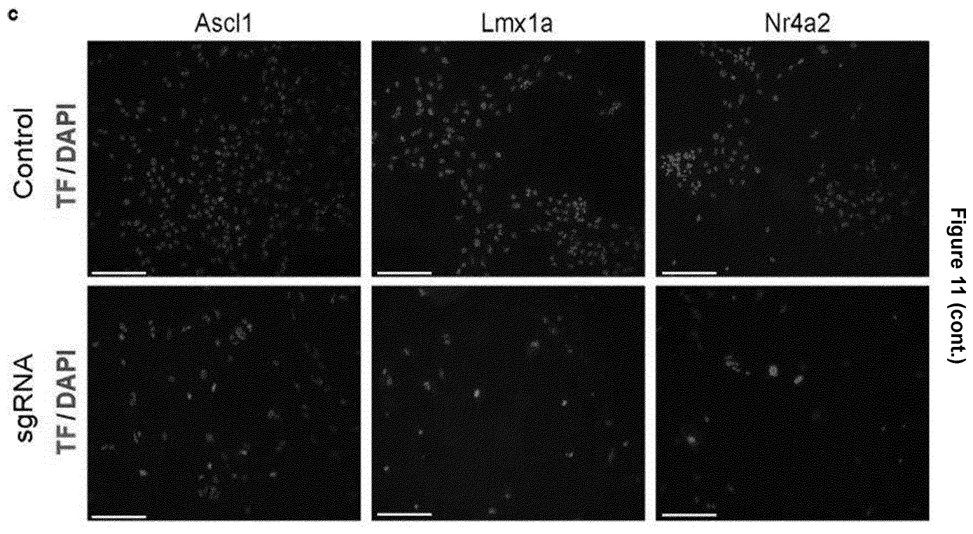

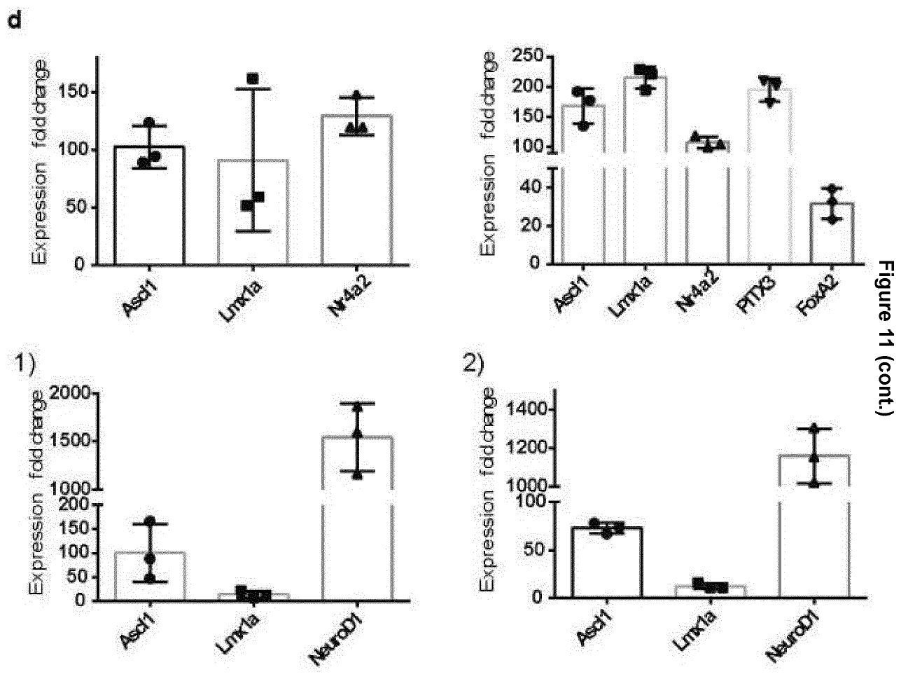

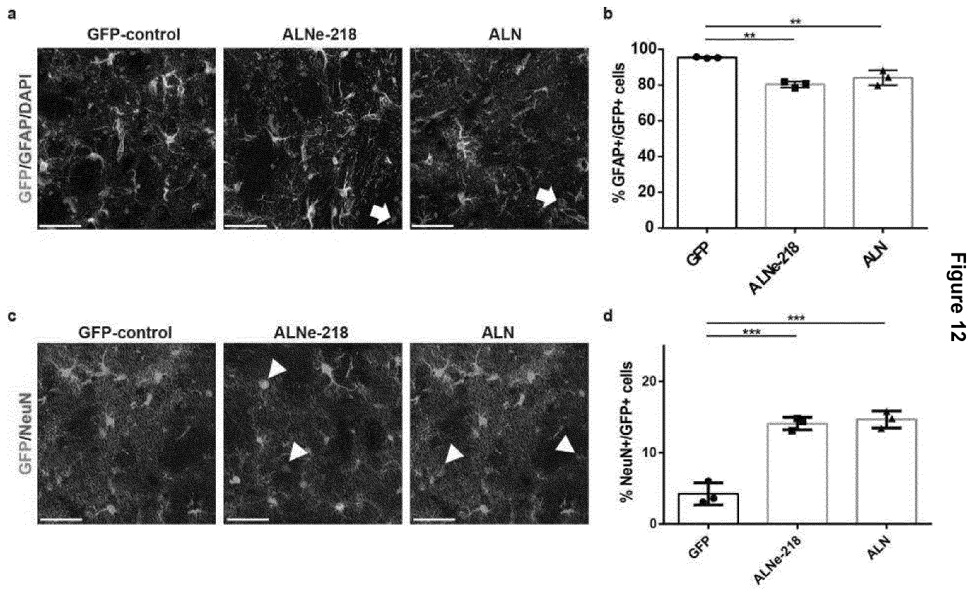

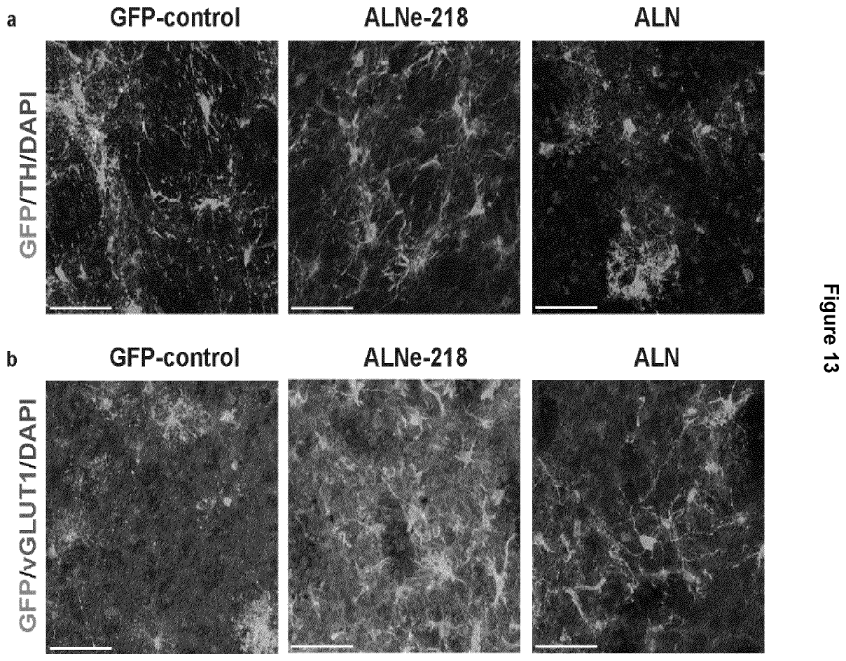

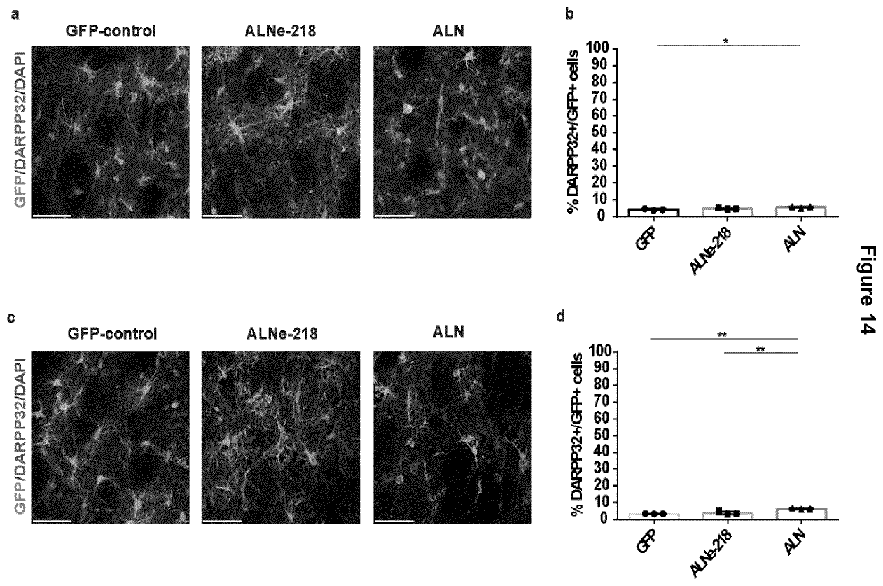

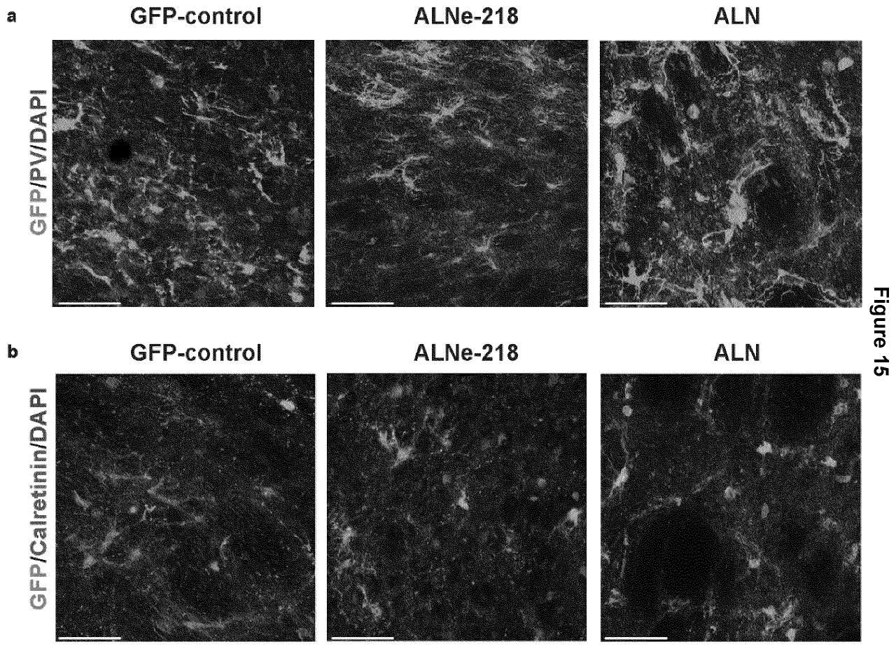

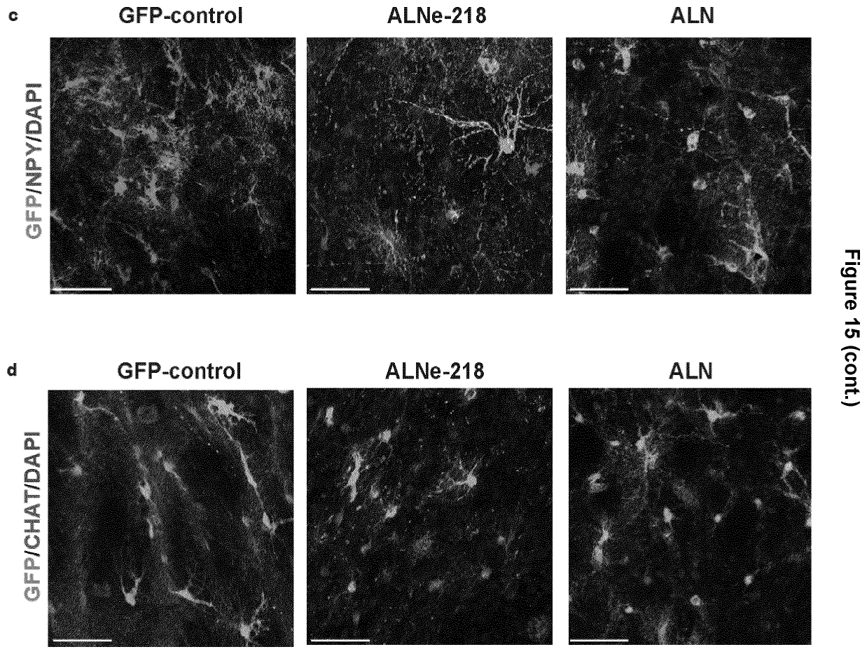

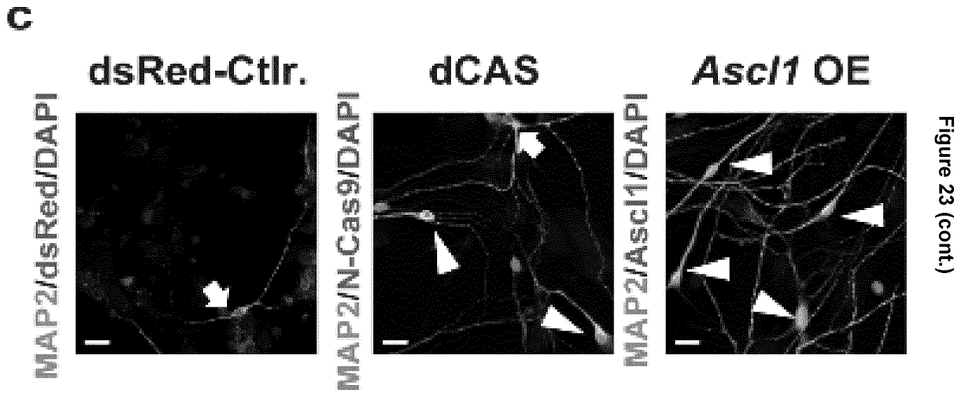

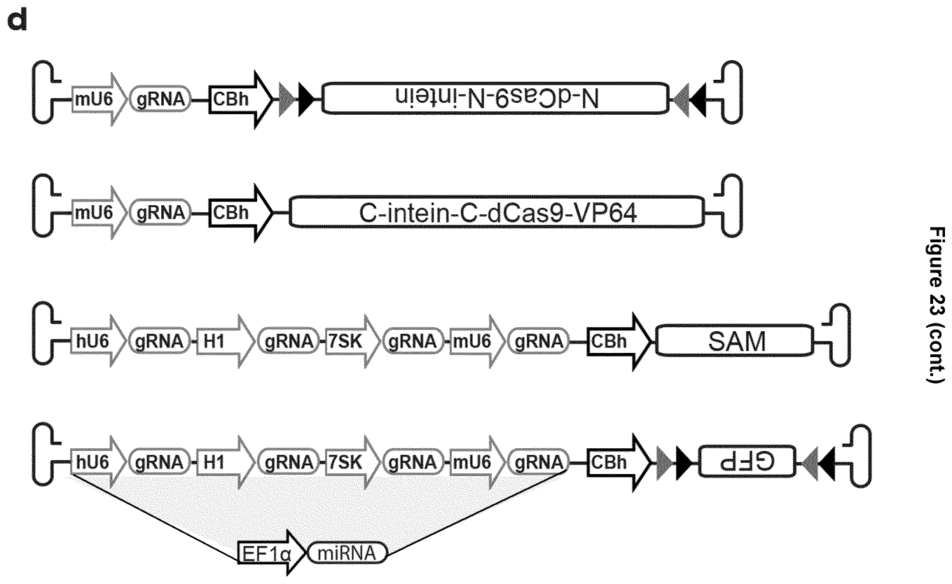

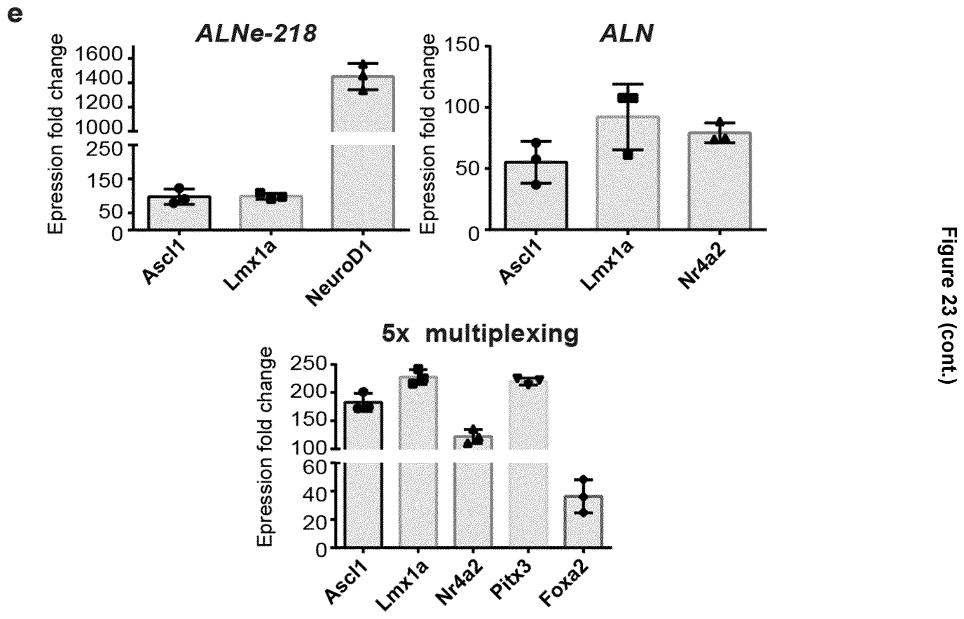

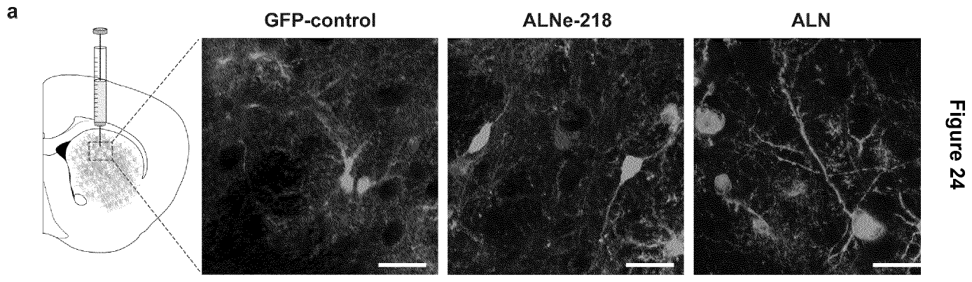

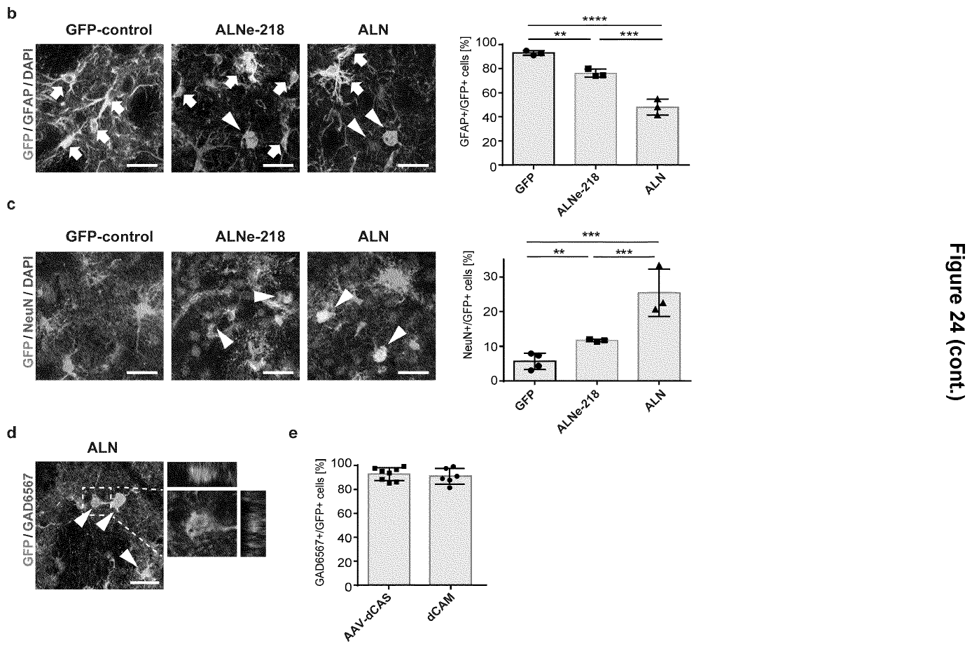

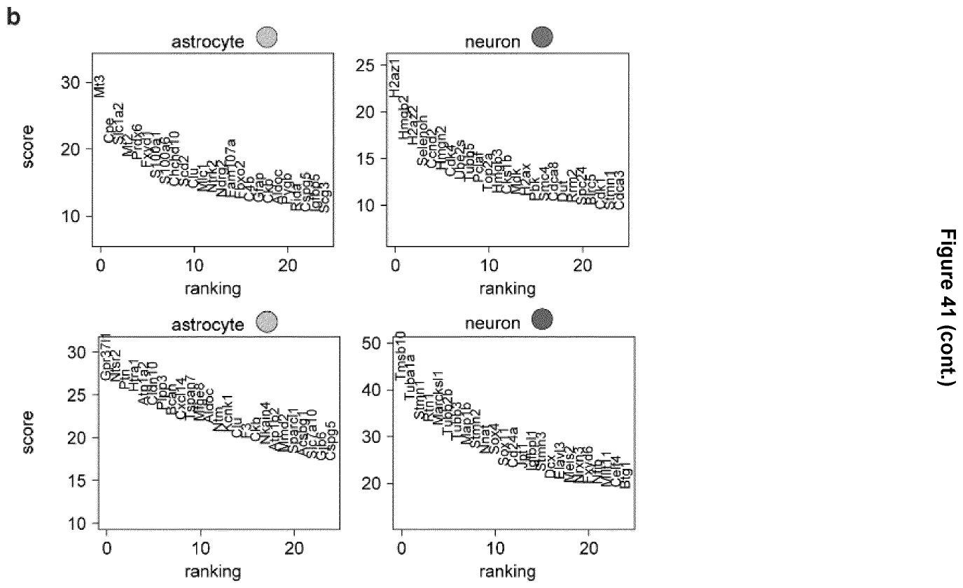

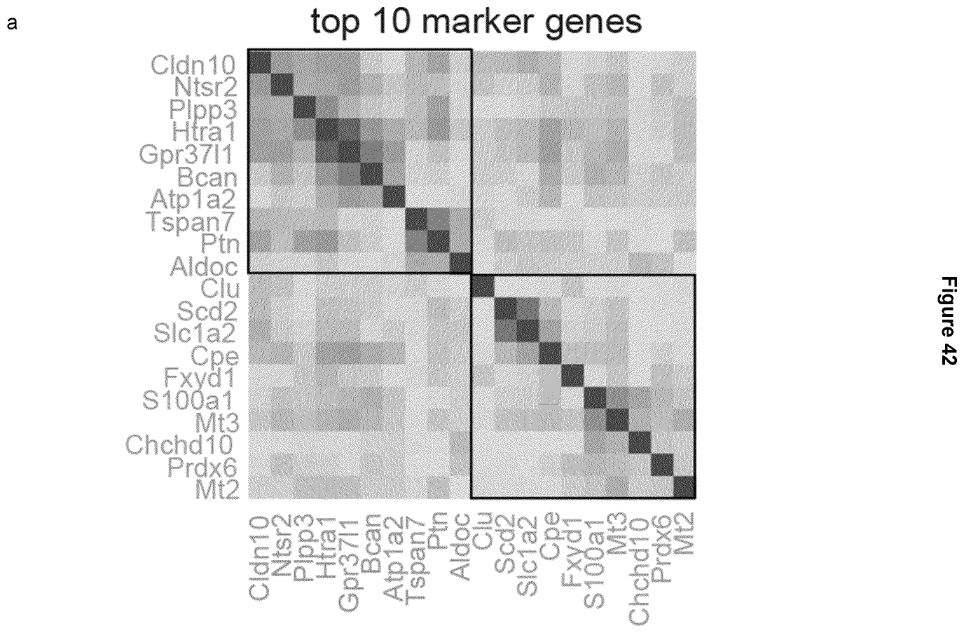

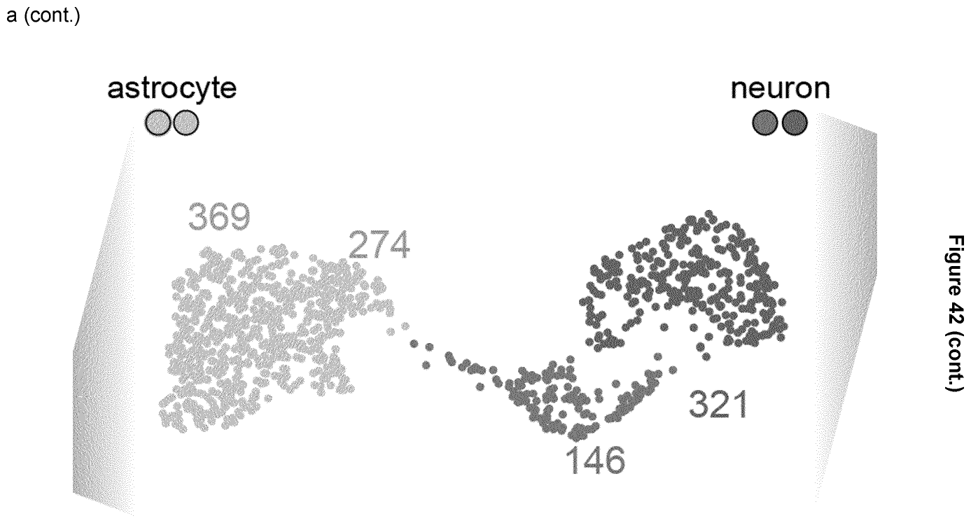

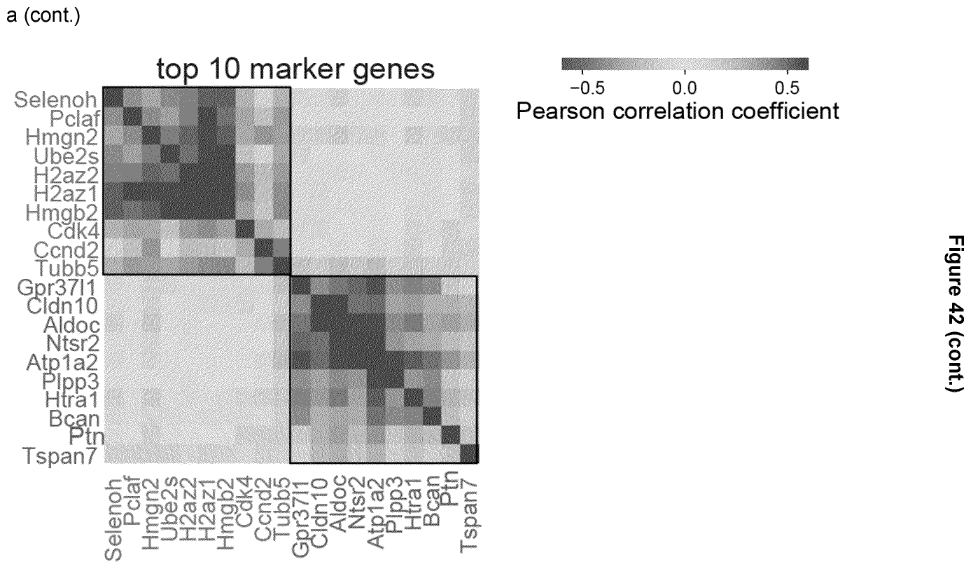

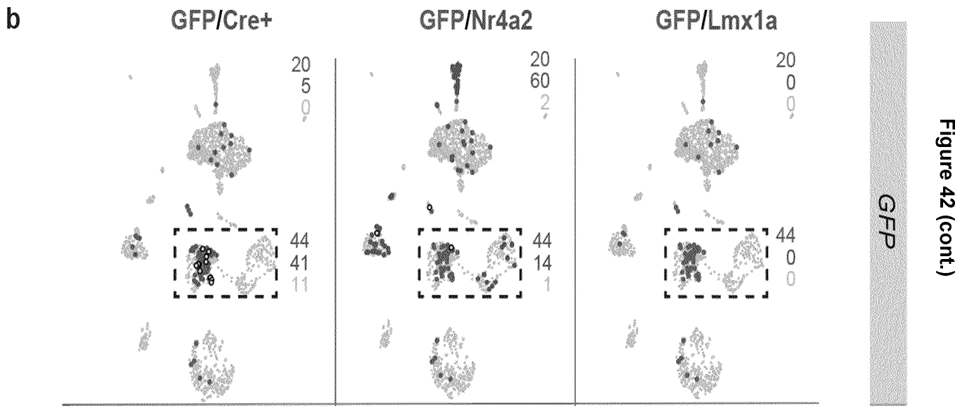

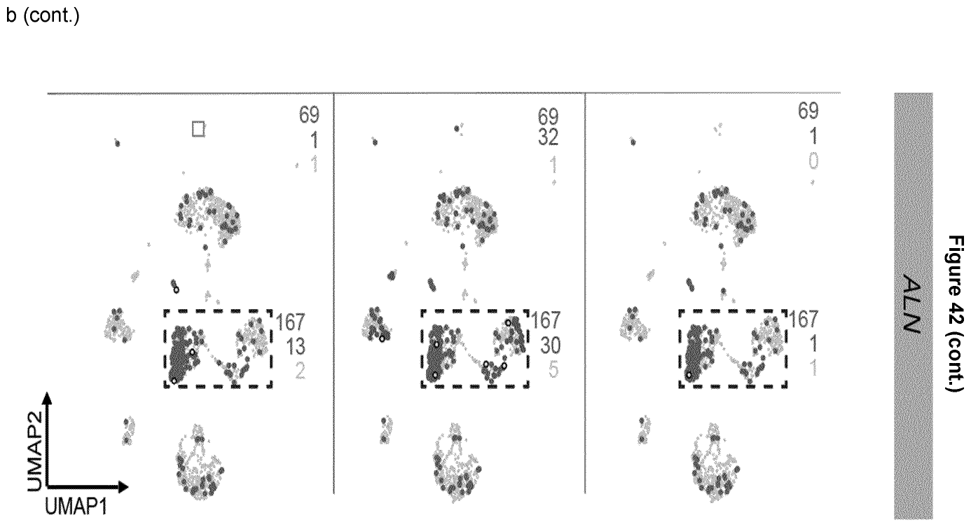

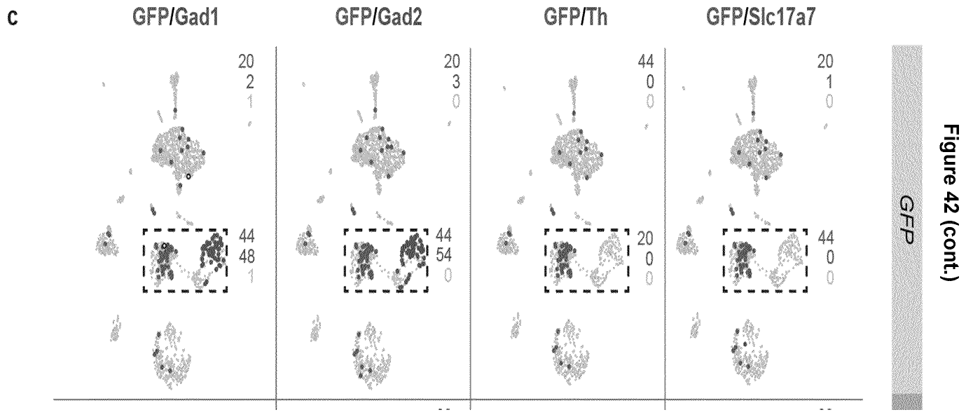

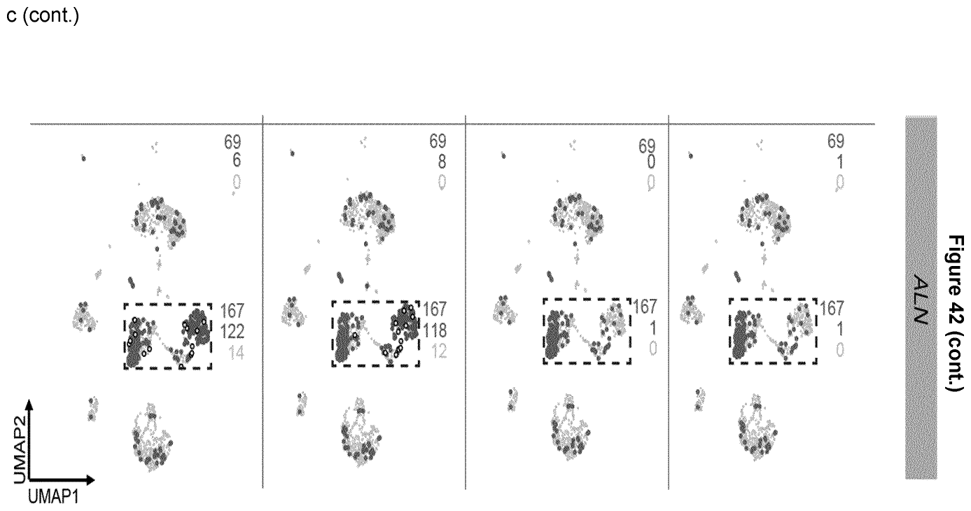

To further characterize the induced neurons scRNA-seq experiments were performed using striatal tissue 13 wpi from GFP control, as well as ALN reprogrammed animals (n=2). The dCAM system was used, as low number of AAVs ensures high consistency within the data. Batch integration of single cell data using Scanorama (Hie et al., 2019) and unsupervised clustering and marker gene annotation of all 3,899 QC-controlled cells (FIG. 16 a-c; also FIG. 40) revealed grouping into main expected striatal cell types such as oligodendrocytes (n=733), astrocytes (n=646), neurons (n=464) and monocytes (n=1,453) (FIG. 3 a; also FIG. 25; FIG. 17; also FIG. 41) (Traag et al., 2019a). Cre expression could be referred to the astrocytic cluster (FIG. 18 b; also FIG. 42). Astrocytic and neuronal cells (n=1,110) were further subclustered, uncovering a total of four cell groups. Selection of marker genes based on cluster-specific up-regulation allowed unsupervised separation of neurons and astrocytes into four subclusters and revealed their cell identities (FIG. 3 b, also FIG. 25; FIG. 18 a; also FIG. 42). In the GFP control condition, besides a small fraction in the oligodendrocyte and monocyte cluster, the majority of GFP+ cells were detected in the astrocyte subclusters. However, solely in the ALN condition, GFP+ cells were mapped to neuronal subclusters as well (n=21, FIG. 3 c; also FIG. 25). Despite the low number of neurons recovered in scRNA-seq experiment versus other cell types (11.2%), we detected expression of all endogenously activated genes (Ascl1, Lmx1a, Nr4a2) co-expressed with GFP in the astrocytic-neuronal subclusters (FIG. 4 c, also FIG. 26; FIG. 18 b; also FIG. 42). Ascl1 expression was enriched in one of the neuronal subcluster, however for the ALN condition, expression could also be detected in one of the astrocytic subclusters (11 out of 17 cells are GFP+ and Asc11+). These Ascii+cells may represent astrocytes with forced expression of endogenous Ascl1, locked in the astrocytic fate or in conversion process. The two neuronal subclusters are characterized by high Ascii or Myt1I expression (FIG. 3 c; also FIG. 25). The analysis for neurotransmitter subtypes revealed no glutamatergic and dopaminergic neurons in the samples, however, the reprogrammed neurons were positive for Gad1/Gad2 confirming a GABAergic fate (14 out of 21 GFP+cells in neuronal cluster are Gad1/2+) (FIG. 3 c, also FIG. 25; FIG. 18 c, also FIG. 42).

### Example 7: Results: Electrophysiological Properties of AAV-dCAS Induced Neurons

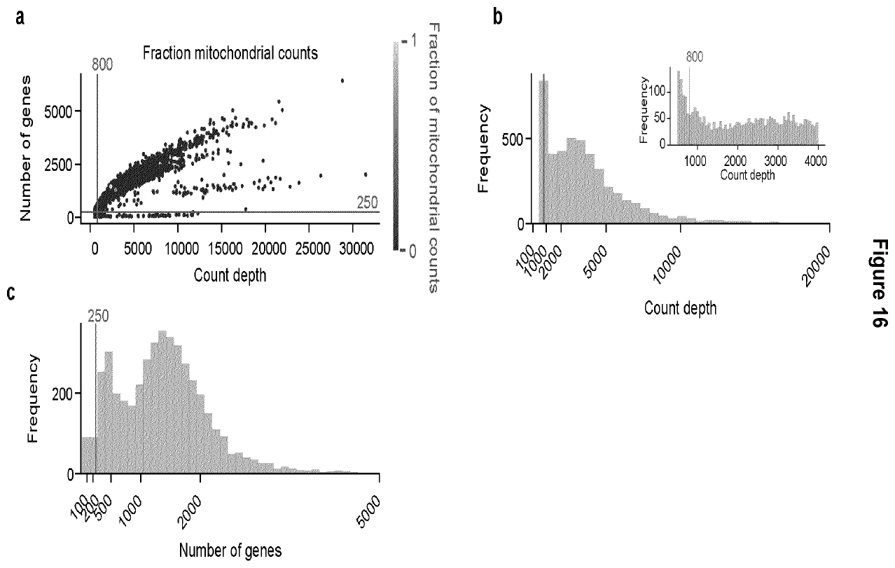

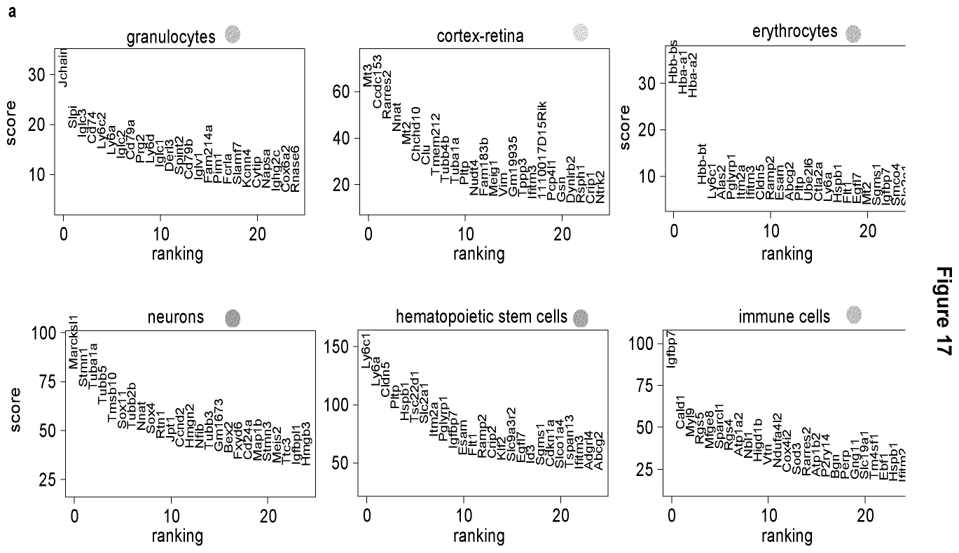

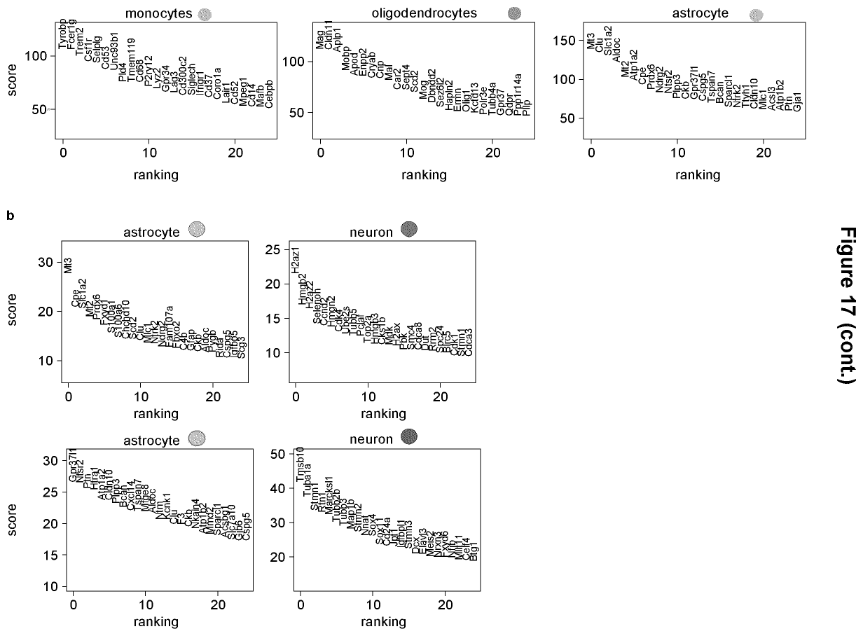

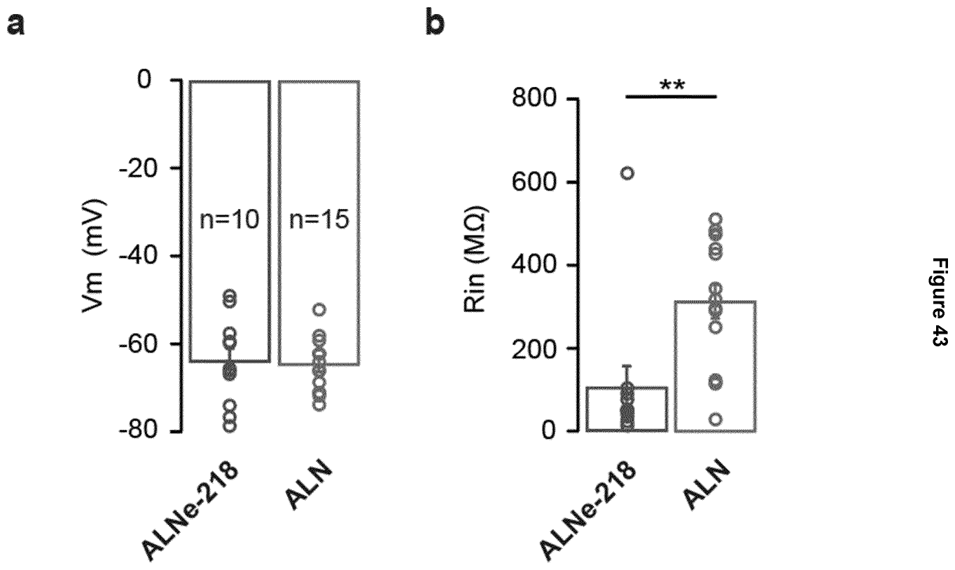

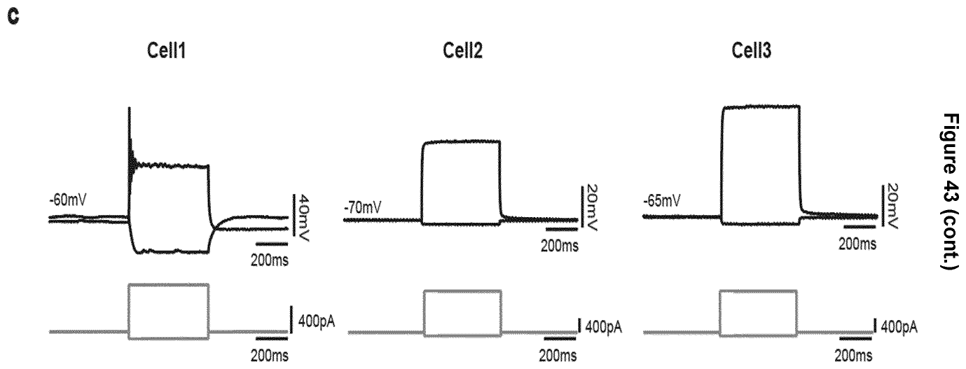

We additionally investigated the electrophysiological properties of neurons reprogrammed with ALN combination 13 weeks after initiating of the reprogramming process and found that induced neurons exhibited mature electrophysiological properties characterized by depolarization-induced action potentials (APs) (FIG. 4 a, AP threshold=-33.49±2.09mV; n=14; also FIG. 26). Further, induced neurons integrated within the striatal network, as shown by the synaptic inputs they received (FIG. 4 a, bottom right; also FIG. 26). Interestingly, ALNe-218-induced neurons displayed properties similar to immature neurons, i.e. the inability to produce APs even with a somatic injection of a strong depolarizing current (>1500 pA) leading to a resting membrane potential above the normal AP threshold observed with the ALN at 13 wpi (FIG. 4 b; also FIG. 26), reminiscent of the characteristics observed at 5 wpi under the ALN condition (FIG. 19; also FIG. 43). No significant difference in the resting membrane potential was observed between these conditions (ALN_{Vm}=−64.55±1.53 mV vs ALNe-218_{Vm}=−63.8±2.99 mV; n=15 and n=10 respectively; p=0.85, Kruskall-Wallis test). However, the input resistance (Rᵢₙ) showed significant differences between the two reprogramming conditions, with a Rᵢₙ for ALN-induced neurons of 314.69±41.2 m0 vs a Rᵢₙ for ALNe-218 neurons of 105.39±52.27 m0 (n=15 and n=10 respectively; p=0.002; Kruskall-Wallis test-FIG. 5 c; also FIG. 28 c), indicating that ALN reprogrammed cells acquire a higher amount of ion channels and a more mature morphology similar to endogenous neurons (Pereira et al., 2017).

### Example 8: Results: ALN-based Reprogramming Rescues Toxin-Induced Motor Phenotype

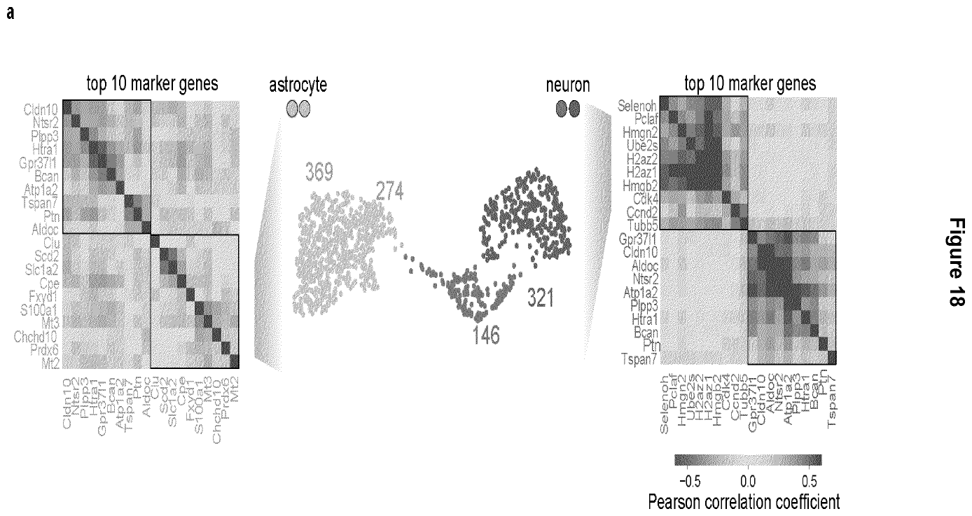

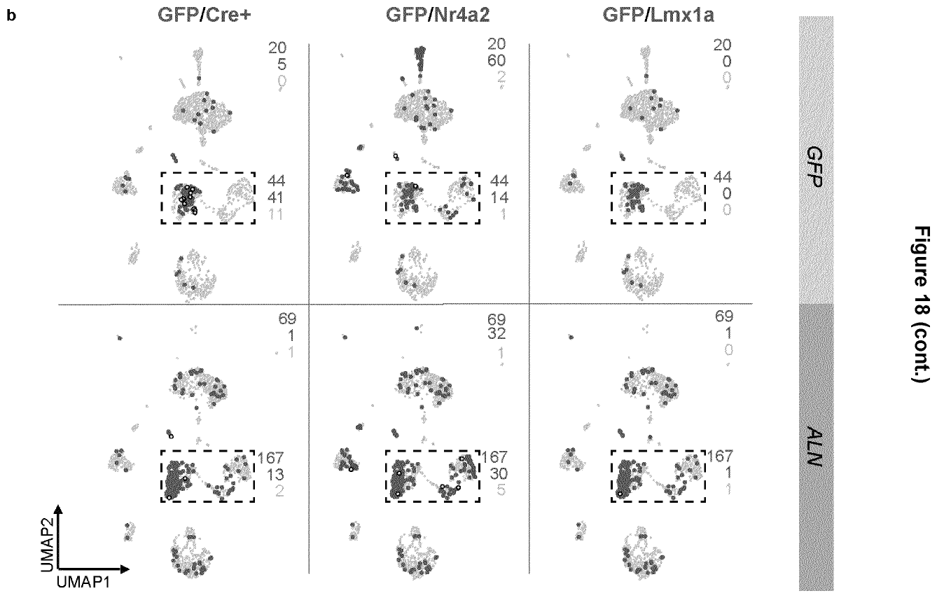

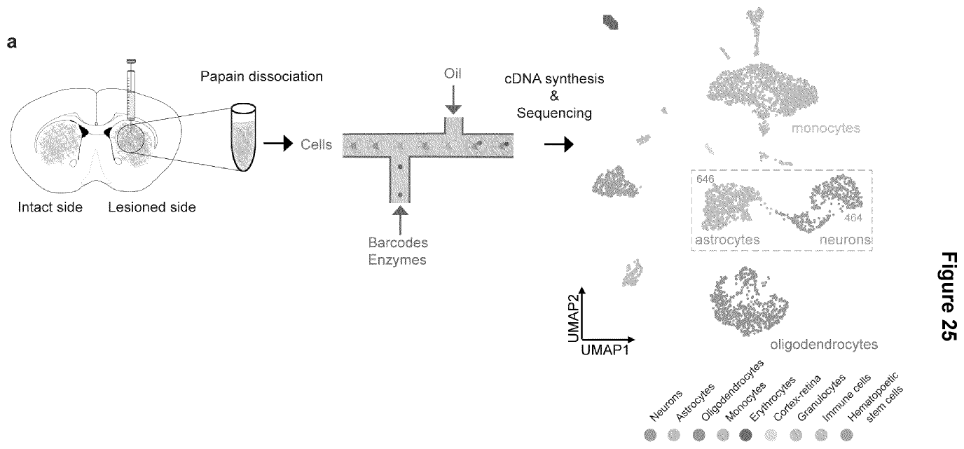

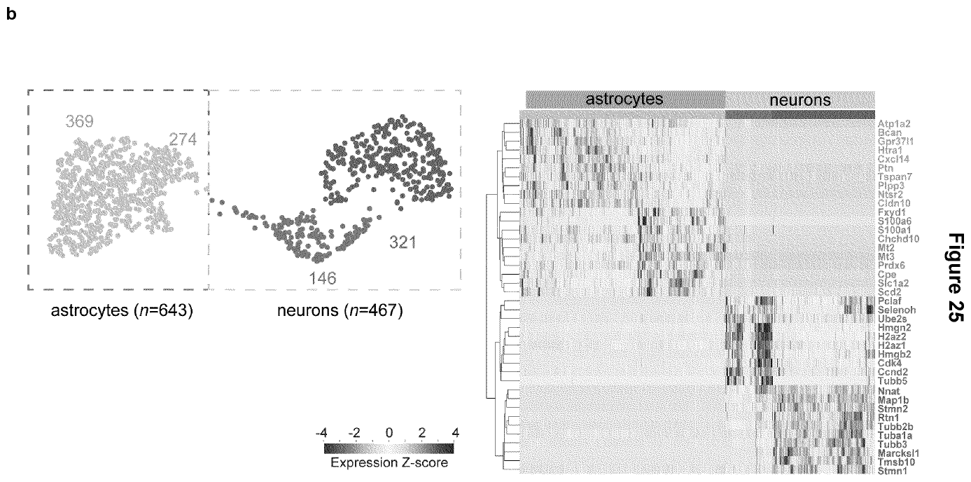

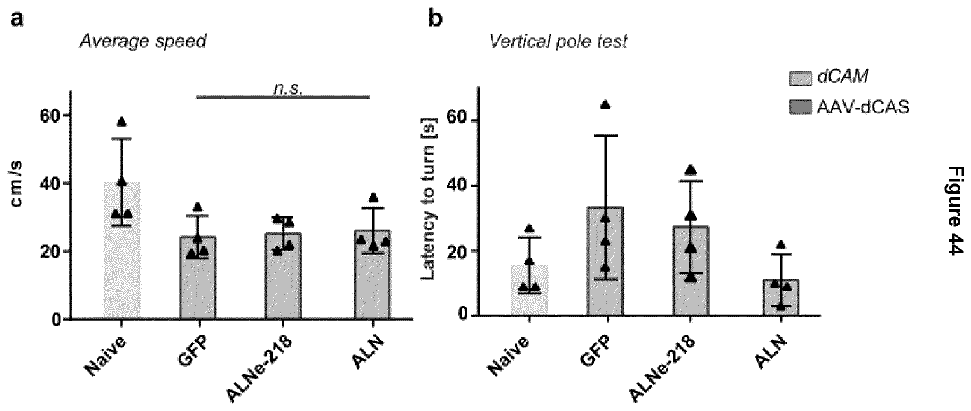

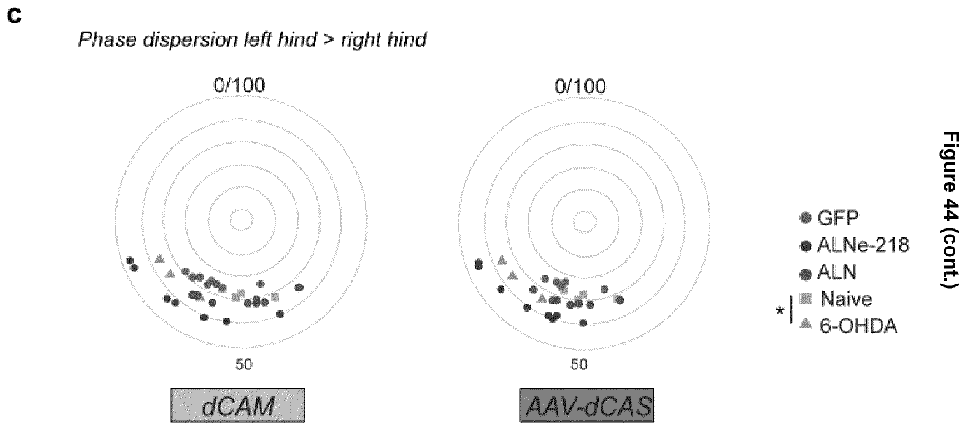

The phenotypical rescue of toxin-induced phenotypes represents the definite proof for the functionality and integration of the induced neurons. We examined the motor behavior in the unilateral 6-OHDA PD model to determine the therapeutic impact of in vivo direct neuronal reprogramming of the dCAM and AAV-dCAS models. Motor behavior was assessed during voluntary movement using the automated CatWalk XT system (Brooks and Dunnett, 2009; Dunnett and Torres, 2012; Glajch et al., 2012; Vandeputte et al., 2010). At 5 wpi we did not observe appreciable differences in spontaneous motor behavior between lesioned animals injected with the activating sgRNA combinations compared to GFP control virus (FIG. 20 a; also FIG. 44). However, at 13 wpi the reprogramming combination ALN induced a significant rescue, demonstrated by the average speed of the animal and in the stride length of the hind paws (FIG. 4 d, e; also FIG. 27). As arm swing is also a parameter altered in PD patients, front paw usage was examined in detail (Mirelman et al., 2016). Accordingly, ipsilateral to the lesion we observed a strong improvement in the duty cycle of the front paw (FIG. 4 f; also FIG. 27). Intriguingly, these very same findings were observed to a similar extent in both systems—using our dCAM knock-in mouse model and the AAV-dCAS setting. Based on these results the dCAM group was additionally analyzed by the vertical pole test, examining striatum-dependent motor coordination, also here a trend towards improved behavior was observed for the ALN combination (FIG. 20 b; also FIG. 44). Importantly, also the coordinated limb usage and axial symmetry, addressed by the phase dispersion between hind paws, converged to naïve levels with the ALN condition (FIG. 20 c; also FIG. 44). Interestingly, when testing for dopamine receptor associated effects of the 6-OHDA PD model, by the assessment of dopamine-dependent drug-induced circling behavior via the amphetamine-induced behavior paradigm, no rescue in rotation behavior was observed in neither condition nor reprogramming model (FIG. 4 g; also FIG. 27). These results confirm that the induced neurons reprogrammed by ALN exhibit functional output leading to a rescue in 6-OHDA motor behavior deficits independent of the dopaminergic system.

Discussion

Parkinson's disease and the associated disturbance in movement coordination and behavior are provoked mainly by the loss of dopaminergic neurons in the SNpc. To date, the prevailing paradigm of disease treatment is the symptomatic management by direct interference of the dopaminergic system. Dopamine levels are restored by drug treatment or through transplantation of dopaminergic neurons (Stoker et al., 2017). As an alternative method, we have developed genetic tools to reprogram striatal astrocytes into mature neurons by the CRISPRa-mediated activation of multiple endogenous transcription factors, such as Ascl1, Lmx1a and Nr4a2 (ALN), or Ascl1, Lmx1a, NeuroD1 together with miRNA218 (ALNe-218) (Caiazzo et al., 2011; Pereira et al., 2017; Rivetti di Val Cervo et al., 2017; Torper et al., 2015). The conventional reprogramming approaches use the ectopic expression of the gene coding sequences (cDNA), making multiplexing of several genes difficult if not impossible, especially when large genes have to be expressed. In contrast, the CRISPRa platform allows multiplexed activation of many endogenous genes solely by introducing specific sgRNAs, with a fixed cargo size for each gene, such that the endogenous transcriptional machinery can be co-opted to execute complex genetic splicing patterns (Pang et al., 2011; Torper et al., 2015; Vierbuchen et al., 2010). Here, we describe two distinct approaches based on CRISPR-mediated gene activation to achieve successful treatment of a murine toxin-induced PD model. For the dCAM mouse line we followed a Rosa26 knock-in strategy of a Cre- and Flpe-dependent dual activator system, harboring the VPR and SAM activator complexes where the defined integration and the optional twofold mode of activation are the prominent features differentiating our line from the recently reported SPH transgenic mouse line (Zhou et al., 2018). After confirming the technical and biological functionality of the dCAM approach, we expanded the toolbox by developing an AAV-based split-dCas9/SAM system, making it versatile and applicable across species with minimal modifications (Truong et al., 2015). Strikingly, with the split-dCas9 AAV-based system (AAV-dCAS) we could recapitulate the results obtained with dCAM, confirming the functionality and robustness of the CRISPRa approach to reprogram striatal astrocytes into induced neurons by multiple gene activation in vivo.

Thirteen weeks after injection, the combination ALN was capable to generate functional neurons with mature electrophysiological properties, whereas cells reprogrammed by ALNe-218 exhibited characteristics reminiscent of astrocytes or immature neurons. Furthermore, only ALN induced neurons led to an improvement in voluntary motor behavior, and a balancing of the axial symmetry. This behavioral rescue could be observed to a similar extent, both in dCAM as well as AAV-dCAS animals, confirming the biological functionality of the CRISPRa-mediated gene activation approach. The de novo induced neurons were not immunoreactive for the dopaminergic marker TH. Nevertheless, independent of reprogramming, we observe TH+ neurons in the striatum, which may either emerge due to the 6-OHDA toxin treatment or represent naturally occurring TH+ interneurons within the striatum (Mao et al., 2019; Pereira et al., 2017; Tepper and Koos, 2016). In this regard, the FLEx-GFP marker employed in this study, proved to be beneficial for the definite identification of induced neurons and its demarcation from reprogramming independent TH+neurons. Interestingly, scRNA-seq analysis of reprogrammed neurons in vivo, as well as immunological staining, revealed a GABAergic identity of the reprogrammed neurons. This is indicating that the regional identity of the targeted astrocytes in combination with the reprogramming factors are predominant for the determination of the final neuronal subtypes, which is supported in part by recent publications of Qian et al. and Zhou et al., 2020 utilizing the knockdown of the RNA-binding protein PTB (Qian et al., 2020; Zhou et al., 2020). The induced neurons were neither positive for DARPP32, a marker for striatal medium spiny neurons representing the main neuronal class within the striatum, nor did they exhibit standard electrophysiological properties of this particular neuronal subtype. This indicates that the reprogrammed neurons presumably differentiate into a distinct GABAergic interneuron population, capable of modulating striatal motor circuits (Cepeda et al., 2008; Gertler et al., 2008; Grande et al., 2013; Planert et al., 2013). Furthermore, these electrophysiological properties are distinct from PV+ interneurons, which have been shown by a recent publication to arise during ALN overexpression in NG2+ oligodendrocyte precursors, which may be explained by the different starter cell populations (Masserdotti et al., 2016; Pereira et al., 2017). The major originality of this study lies in the fact, that the CRISPRa induced ALN combination in the striatum, using either dCAM or AAV-dCAS, induces specific GABAergic neurons, capable of alleviating motor behavior symptoms in a 6-OHDA model. This is surprising, since the research focus so far has been on the restoration of the dopaminergic drive to alleviate motor symptoms. However, it has been reported that dopamine depletion in 6-OHDA toxin treated PD rodent models has a strong effect on striatal circuits. Specifically, increased excitatory cholinergic and reduced inhibitory GABAergic signals have been observed (Salin et al., 2009). In addition, most of the basal striatal excitatory drive arising from cholinergic interneurons is balanced by a concomitant GABAergic inhibition; this signaling is impaired by dopamine deprivation (Lozovaya et al., 2018). Furthermore, integrity of the fast spiking striatal GABAergic interneurons has been shown to depend on dopaminergic input from SNpc39. Altogether, these reports as well as our own findings suggest, that the imbalance in striatal microcircuitry-including impaired GABAergic signaling-contribute to the altered motor behavior in parkinsonian state. These observations are supported by Martinez-Cerdeno et al. transplanting GABAergic neuron precursors into the striatum of parkinsonian state rats (Martinez-Cerdeno et al., 2010) and also rescuing in part motor behavior. Therefore, restoration or reinforcing of GABAergic inhibition in the striatum is an attractive alternative therapeutic concept for PD beyond the dopamine replacement strategies. Future experiments will show which strategy will reveal the best therapeutic effects.

In summary, here we show, that the transgene dCAM and the universal applicable AAV-dCAS system can rescue PD motor behavior deficits by the direct conversion of endogenous astrocytes into functional GABAergic neurons via a CRISPRa mediated induction of the reprogramming factors Ascl1, Lmx1a and Nr4a2. These novel tools can be employed to any reprogramming approaches in any organ, tissue and cell-type in vivo.

### REFERENCES

Berninger, B., et al., 2007. Functional properties of neurons derived from in vitro reprogrammed postnatal astroglia. J Neurosci. 27, 8654-64.
Breunig, C. T., et al., 2018. One step generation of customizable gRNA vectors for multiplex CRISPR approaches through string assembly gRNA cloning (STAgR). PLoS One. 13, e0196015.
Brooks, S. P., Dunnett, S. B., 2009. Tests to assess motor phenotype in mice: a user's guide. Nat Rev Neurosci. 10, 519-29.
Caiazzo, M., et al., 2011. Direct generation of functional dopaminergic neurons from mouse and human fibroblasts. Nature. 476, 224-7.
Cepeda, C., et al., 2008. Differential electrophysiological properties of dopamine D1 and D2 receptor-containing striatal medium-sized spiny neurons. Eur J Neurosci. 27, 671-82.
Chavez, A., et al., 2015. Highly efficient Cas9-mediated transcriptional programming. Nat Methods. 12, 326-8.
Colasante, G., et al., 2020. In vivo CRISPRa decreases seizures and rescues cognitive deficits in a rodent model of epilepsy. Brain. 143, 891-905.
D′Costa, S., et al., 2016. Practical utilization of recombinant AAV vector reference standards: focus on vector genomes titration by free ITR qPCR. Mol Ther Methods Clin Dev. 5, 16019.
Dunnett, S. B., Torres, E.M., 2012. Rotation in the 6-OHDA-Lesioned Rat. In: Animal Models of Movement Disorders: Volume I. Vol., E. L. Lane, S. B. Dunnett, ed.Aeds. Humana Press, Totowa, NJ, pp. 299-315.
Foglieni, C., et al., 2017. Split GFP technologies to structurally characterize and quantify functional biomolecular interactions of FTD-related proteins. Sci Rep. 7, 14013.
Gertler, T. S., Chan, C. S., Surmeier, D. J., 2008. Dichotomous anatomical properties of adult striatal medium spiny neurons. J Neurosci. 28, 10814-24.
Glajch, K. E., et al., 2012. Sensorimotor assessment of the unilateral 6-hydroxydopamine mouse model of Parkinson's disease. Behav Brain Res. 230, 309-16.
Grande, A., et al., 2013. Environmental impact on direct neuronal reprogramming in vivo in the adult brain. Nature communications. 4, 1-12.
Grealish, S., et al., 2010. Characterisation of behavioural and neurodegenerative changes induced by intranigral 6-hydroxydopamine lesions in a mouse model of Parkinson's disease. Eur J Neurosci. 31, 2266-78.
Gregorian, C., et al., 2009. Pten deletion in adult neural stem/progenitor cells enhances constitutive neurogenesis. J Neurosci. 29, 1874-86.
Grieger, J. C., Samulski, R.J., 2005. Packaging capacity of adeno-associated virus serotypes: impact of larger genomes on infectivity and postentry steps. J Virol. 79, 9933-44.
Guo, Z., et al., 2014. In vivo direct reprogramming of reactive glial cells into functional neurons after brain injury and in an Alzheimer's disease model. Cell Stem Cell. 14, 188-202.
Heinrich, C., et al., 2010. Directing astroglia from the cerebral cortex into subtype specific functional neurons. PLoS Biol. 8, e1000373.
Heinrich, C., et al., 2011. Generation of subtype-specific neurons from postnatal astroglia of the mouse cerebral cortex. Nat Protoc. 6, 214-28.
Heins, N., et al., 2002. Glial cells generate neurons: the role of the transcription factor Pax6. Nat Neurosci. 5, 308-15.
Hie, B., Bryson, B., Berger, B., 2019. Efficient integration of heterogeneous single-cell transcriptomes using Scanorama. Nat Biotechnol. 37, 685-691.
Hölter, S. M., Glasl, L., 2012. High-Throughput Mouse Phenotyping. In: Animal Models of Movement Disorders: Volume I. Vol., E. L. Lane, S. B. Dunnett, ed.Aeds. Humana Press, Totowa, NJ, pp. 109-133.
Jamebozorgi, K., et al., 2019. Cellular and molecular aspects of Parkinson treatment: future therapeutic perspectives. Molecular neurobiology. 56, 4799-4811.
Konermann, S., et al., 2015. Genome-scale transcriptional activation by an engineered CRISPR-Cas9 complex. Nature. 517, 583-8.
Lozovaya, N., et al., 2018. GABAergic inhibition in dual-transmission cholinergic and GABAergic striatal interneurons is abolished in Parkinson disease. Nat Commun. 9, 1422.
Luecken, M. D., Theis, F. J., 2019. Current best practices in single-cell RNA-seq analysis: a tutorial. Mol Syst Biol. 15, e8746.
Mao, M., Nair, A., Augustine, G. J., 2019. A Novel Type of Neuron Within the Dorsal Striatum. Front Neural Circuits. 13, 32.
Martinez-Cerdeno, V., et al., 2010. Embryonic MGE precursor cells grafted into adult rat striatum integrate and ameliorate motor symptoms in 6-OHDA-lesioned rats. Cell Stem Cell. 6, 238-50.
Masserdotti, G., Gasc6n, S., Götz, M., 2016. Direct neuronal reprogramming: learning from and for development. Development. 143, 2494-2510.
Mattugini, N., et al., 2019. Inducing Different Neuronal Subtypes from Astrocytes in the Injured Mouse Cerebral Cortex. Neuron. 103, 1086-1095 e5.
McGregor, M. M., Nelson, A. B., 2019. Circuit Mechanisms of Parkinson's Disease. Neuron. 101, 1042-1056.
Melville, J., McInnes, L., Healy, J., 2018. UMAP: uniform manifold approximation and projection for dimension reduction. Preprint at arXiv https://arxiv. org/abs/1802.03426.
Mirelman, A., et al., 2016. Arm swing as a potential new prodromal marker of Parkinson's disease. Mov Disord. 31, 1527-1534.
Pang, Z. P., et al., 2011. Induction of human neuronal cells by defined transcription factors. Nature. 476, 220-3.
Parmar, M., Grealish, S., Henchcliffe, C., 2020. The future of stem cell therapies for Parkinson disease. Nat Rev Neurosci. 21, 103-115.
Pereira, M., et al., 2017. Direct Reprogramming of Resident NG2 Glia into Neurons with Properties of Fast-Spiking Parvalbumin-Containing Interneurons. Stem Cell Reports. 9, 742-751.
Planert, H., Berger, T. K., Silberberg, G., 2013. Membrane properties of striatal direct and indirect pathway neurons in mouse and rat slices and their modulation by dopamine. PLoS One. 8, e57054.
Qian, H., et al., 2020. Reversing a model of Parkinson's disease with in situ converted nigral neurons. Nature. 582, 550-556.
Rivetti di Val Cervo, P., et al., 2017. Induction of functional dopamine neurons from human astrocytes in vitro and mouse astrocytes in a Parkinson's disease model. Nat Biotechnol. 35, 444-452.
Salin, P., et al., 2009. Changes to interneuron-driven striatal microcircuits in a rat model of Parkinson's disease. Neurobiol Dis. 34, 545-52.
Schlachetzki, J.C., et al., 2014. Increased tyrosine hydroxylase expression accompanied by glial changes within the non-lesioned hemisphere in the 6-hydroxydopamine model of Parkinson's disease. Restor Neurol Neurosci. 32, 447-62.
Stoker, T. B., Blair, N. F., Barker, R. A., 2017. Neural grafting for Parkinson's disease: challenges and prospects. Neural Regen Res. 12, 389-392.
Tepper, J., Koos, T., 2016. Gabaergic interneurons of the Striatum. In: Handbook of Behavioral Neuroscience. Vol. 24, ed.Aeds. Elsevier, pp. 157-178.
Theodorou, M., et al., 2015. Limitations of In Vivo Reprogramming to Dopaminergic Neurons via a Tricistronic Strategy. Hum Gene Ther Methods. 26, 107-22.
Torper, O., et al., 2015. In Vivo Reprogramming of Striatal NG2 Glia into Functional Neurons that Integrate into Local Host Circuitry. Cell Rep. 12, 474-81.
Traag, V. A., Waltman, L., van Eck, N.J ., 2019a. From Louvain to Leiden: guaranteeing well-connected communities. Scientific Reports. 9, 5233.
Traag, V. A., Waltman, L., van Eck, N. J., 2019b. From Louvain to Leiden: guaranteeing well-connected communities. Sci Rep. 9, 5233.
Truong, D. J., et al., 2015. Development of an intein-mediated split-Cas9 system for gene therapy. Nucleic Acids Res. 43, 6450-8.
Vandeputte, C., et al., 2010. Automated quantitative gait analysis in animal models of movement disorders. BMC Neuroscience. 11, 92.
Vierbuchen, T., et al., 2010. Direct conversion of fibroblasts to functional neurons by defined factors. Nature. 463, 1035-41.
Vignoles, R., et al., 2019. Direct Lineage Reprogramming for Brain Repair: Breakthroughs and Challenges. Trends Mol Med. 25, 897-914.
Wolf, F. A., Angerer, P., Theis, F.J., 2018. SCANPY: large-scale single-cell gene expression data analysis. Genome Biol. 19, 15.
Zaiss, A. K., Muruve, D.A., 2005. Immune responses to adeno-associated virus vectors. Curr Gene Ther. 5, 323-31.
Zhou, H., et al., 2018. In vivo simultaneous transcriptional activation of multiple genes in the brain using CRISPR-dCas9-activator transgenic mice. Nat Neurosci. 21, 440-446.
Zhou, H., et al., 2020. Glia-to-Neuron Conversion by CRISPR-CasRx Alleviates Symptoms of Neurological Disease in Mice. Cell. 181, 590-603 e16.
Zimprich, A., et al., 2018. Analysis of locomotor behavior in the German Mouse Clinic. J Neurosci Methods. 300, 77-91.
Zolotukhin, S., et al., 1999. Recombinant adeno-associated virus purification using novel methods improves infectious titer and yield. Gene Ther. 6, 973-85.

One skilled in the art would readily appreciate that the present invention is well adapted to carry out the objects and obtain the ends and advantages mentioned, as well as those inherent therein. Further, it is readily apparent to one skilled in the art that varying substitutions and modifications may be made to the invention disclosed herein without departing from the scope and spirit of the invention. The compositions, methods, procedures, treatments, molecules and specific compounds described herein are presently representative of certain embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing“, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied herein may be resorted to by those skilled in the art, and that such modifications and variations are considered to be within the scope of this invention.

The invention has been described broadly and generically herein. Each of the narrower species and sub generic groupings falling within the generic disclosure also form part of the invention. This includes the generic description of the invention with a proviso or negative limitation removing any subject matter from the genus, regardless of whether or not the excised material is specifically recited herein. All documents, including patent applications and scientific publications, referred to herein are incorporated herein by reference for all purposes.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A plurality of separate adeno-associated viruses (AAVs) comprising:
(i) a first AAV comprising a first nucleic acid encoding a first portion of a Cas9 protein devoid of endonuclease activity;
preferably said first nucleic acid further encoding a first split-intein polypeptide (e.g., an N-intein polypeptide), further preferably said first split-intein polypeptide having at least 80% sequence identity with the polypeptide having SEQ ID NO: 10 (N-Split-lntein) and having the intein activity (e.g., protein trans-splicing activity);
most preferably said first portion of said Cas9 protein devoid of said endonuclease activity is devoid of an enzymatic activity having EC: 3.1.-further most preferably said first nucleic acid encoding the polypeptide having at least 80% sequence identity with the polypeptide having SEQ ID NO: 2 (N-dCas9-N-intein);
optionally said first nucleic acid further encoding one or more guide RNAs (gRNAs) (e.g., said first nucleic acid is about 4.5 Kb in size);
(ii) a second AAV comprising a second nucleic acid encoding a second portion of a Cas9 protein devoid of endonuclease activity;
preferably said second nucleic acid further encoding a second split-intein polypeptide having complementarity to said first split-intein (e.g., a C-intein), further preferably said second split-intein polypeptide having at least 80% sequence identity with the polypeptide having SEQ ID NO: 12 (C-Split-lntein) and having the intein activity (e.g., protein trans-splicing activity);
most preferably said second nucleic acid further encoding at least one polypeptide having a trans-activating activity (e.g., activating transcription) and/or one or more guide RNAs (gRNAs),; further most preferably said at least one polypeptide having trans-activating activity having at least 80% sequence identity with the polypeptide having SEQ ID NO: 20 (VP16);
further most preferably said second portion of said Cas9 protein devoid of said endonuclease activity is devoid of an enzymatic activity having EC: 3.1.-.-.;
further most preferably said second nucleic acid encoding the polypeptide having at least 80% sequence identity with the polypeptide having SEQ ID NO: 4 (C-dCas9-C-intein-VP64);
(iii) a third AAV comprising a third nucleic acid encoding at least one polypeptide having a trans-activating activity (e.g., activating transcription) and capable of binding to and/or associating with an at least one guide RNA (gRNA) and/or said first and/or second portion of said Cas9 protein, wherein said third nucleic acid further encoding at least one guide RNA (gRNA) comprising at least one aptamer capable of binding at least one transcription co-activator protein (e.g., said co-activator protein comprising one or more of the following: (i) MS2 adaptor polypeptide having SEQ ID NO: 14; (ii) MS2-p65 polypeptide having SEQ ID NO: 16; and/or (iii) MS2-p65-HSF1 polypeptide having SEQ ID NO: 18), preferably said third nucleic acid encoding a synergistic activation mediator (SAM) complex and at least one guide RNA (gRNA) comprising at least one aptamer capable of binding at least one co-activator protein (e.g., said co-activator protein comprising one or more of the following: (i) MS2 adaptor polypeptide having SEQ ID NO: 14; (ii) MS2-p65 polypeptide having SEQ ID NO: 16; and/or (iii) MS2-p65-HSF1 polypeptide having SEQ ID NO: 18), most preferably comprising at least two aptamers, each capable of binding at least two co-activator proteins (e.g., said co-activator protein comprising one or more of the following: (i) MS2 adaptor polypeptide having SEQ ID NO: 14; (ii) MS2-p65 polypeptide having SEQ ID NO: 16; and/or (iii) MS2-p65-HSF1 polypeptide having SEQ ID NO: 18),
(iv) optionally a fourth AAV comprising a fourth nucleic acid encoding a reporter polypeptide, preferably said reporter polypeptide is a fluorescent protein, further preferably said fluorescent protein is a green fluorescence protein;
wherein said first portion of said Cas9 protein devoid of endonuclease activity and said second portion of said Cas9 protein devoid of endonuclease activity, when joined together, form a Cas9 protein devoid of endonuclease activity, preferably said formed Cas9 protein is capable of binding DNA.

2. The plurality of claim 1, wherein the first portion of the Cas9 protein is the N-terminal lobe of the Cas9 protein and the second portion of the Cas9 protein is the C-terminal lobe of the Cas9 protein.

3. The plurality of any one of the preceding claims, wherein:
(i) the first portion of the Cas9 protein is the N-terminal lobe of the Cas9 protein up to amino acid 573 and the second portion of the Cas9 protein is the C-terminal lobe of the Cas9 protein beginning at amino acid 574; or
(ii) the first portion of the Cas9 protein is the N-terminal lobe of the Cas9 protein up to amino acid 637 and the second portion of the Cas9 protein is the C-terminal lobe of the Cas9 protein beginning at amino acid 638.

4. The plurality of any one of the preceding claims, wherein the first nucleic acid encodes a first portion of the Cas9 protein having a first split-intein and wherein the second nucleic acid encodes a second portion of the Cas9 protein having a second split-intein complementary to the first split-intein, wherein the first portion of the Cas9 protein and the second portion of the Cas9 protein, when joined together, form the Cas9 protein (e.g., by intein-mediated protein trans-splicing).

5. The plurality of any one of the preceding claims, wherein the first nucleic acid encodes a first portion of the Cas9 protein having a Nostoc punctiforme (Npu) strain PCC73102 N-split-intein IntN and wherein the second nucleic acid encodes a second portion of the Cas9 protein having a Nostoc punctiforme (Npu) strain PCC73102 C-split-intein IntC, wherein the first portion of the Cas9 protein and the second portion of the Cas9 protein, when joined together, form the Cas9 protein.

6. The plurality of any one of the preceding claims, wherein the SAM complex comprises a MS2 coat protein (e.g., having SEQ ID NO: 14) fused to the p65 subunit of NF-kappaB (e.g., forming SEQ ID NO: 16) and the activation domain of human heat-shock factor 1 (HSF1) (e.g., forming SEQ ID NO: 18).

7. The plurality of any one of the preceding claims, wherein a second portion of a Cas9 protein devoid of endonuclease activity is fused to a transcription activation domain, preferably said transcription activation domain having at least 80% sequence identity with the polypeptide having SEQ ID NO: 20 (VP16).

8. The plurality of any one of the preceding claims, wherein the third nucleic acid further encodes a transcription activation domain.

9. The plurality of claim 7 or 8, wherein said transcription activation domain comprising at least one VP16 (SEQ ID NO: 20) domain, preferably comprising a quadruple VP16 (VP64) domain.

10. The plurality of any one of the preceding claims, wherein the Cas9 is a Type II CRISPR system Cas9.

11. A composition, kit, expression system or recombinant host cell (e.g., isolated) comprising the plurality of any one of the preceding claims, preferably said composition, kit, expression system or recombinant host cell is pharmaceutical and/or diagnostic composition, kit, expression system or recombinant host cell, further preferably said composition, kit, expression system or recombinant host cell further comprising a reporter, most preferably said reporter is a fluorescent protein, further most preferably said fluorescent protein is a green fluorescence protein.

12. The plurality, composition, kit, expression system or recombinant host cell of any one of the preceding claims for use as a medicament and/or in therapy.

13. A method for reprogramming and/or modifying a cell, said method comprising:
a) providing: (i) a cell; and (ii) the plurality, composition, kit, expression system or recombinant host cell of any one of the preceding claims;
b) applying and/or expressing (ii) to/in (i);
preferably said cell is an astrocyte, further preferably said cell is reprogrammed into a neuron, most preferably said astrocyte is reprogrammed into a neuron.

14. The plurality, composition, kit, expression system or recombinant host cell of any one of the preceding claims, for use in one or more of the following methods:
i) method of treatment, amelioration, prophylaxis and/or diagnostics of a neurodegenerative disease, cancer, cardiovascular disease, metabolic disease, monogenic disorder (e.g., single-gene associated disorder, e.g., Osteogenesis Imperfecta (OGI), Retinoblastoma (RB), Cystic Fibrosis, Thalassemia, Fragile X Syndrome (FXS), Hypophosphatemia, Hemophilia and Ichthyosis) and/or diabetes, preferably said neurodegenerative disease is selected from the group consisting of: Parkinson's disease, Parkinsonism, Parkinson-plus syndrome, Alzheimer's disease, Amyotrophic lateral sclerosis (ALS) and Huntington's disease;
ii) method for reprogramming and/or modifying a cell, preferably an astrocyte, further preferably into a neuron;
iii) method for inducing and/or modifying expression of one or more genes of interest (e.g., endogenous, e.g., transcription factors, e.g., one or more of the following: Achaete-scute homolog 1 (Ascl1, e.g., UniProtKB-P50553 or Q02067), LIM homeobox transcription factor 1-alpha (Lmx1a, e.g., UniProtKB-Q8TE12 or Q9JKU8), Nurr1 (e.g., UniProtKB -P43354), preferably or alternatively Achaete-scute homolog 1 (Ascl1, e.g., UniProtKB-P50553 or Q02067), LIM homeobox transcription factor 1-alpha (Lmx1a, e.g., UniProtKB-Q8TE12 or Q9JKU8), Neurogenic differentiation factor 1 (Neurod1, e.g., UniProtKB-Q13562 or Q60867), miRNA218 (e.g., NR_029632, NR_029799.1);
iv) method for cell-replacement and/or transplantation;
v) method for somatic reprogramming of a cell; preferably an astrocyte, further preferably into a neuron;
vi) method for genome and /or transcriptome modification, and/or gene therapy;
vii) method of screening (e.g., guide RNAs) and/or monitoring gene expression; preferably said composition or kit further comprising a reporter, further preferably said reporter is a fluorescent protein, further preferably said fluorescent protein is a green fluorescence protein;
viii) method for producing a neuron;
viii) in a method according to any one of preceding claims;
ix) said method is an in vitro, in vivo or ex vivo method;
x) in any combination of (i)-(x).

15. Use of the plurality, composition, kit, expression system or recombinant host cell of any one of the preceding claims, for one or more of the following:
i) for reprogramming and/or modifying a cell, preferably an astrocyte, further preferably into a neuron;
ii) for inducing and/or modifying expression of one or more genes of interest (e.g., endogenous, e.g., transcription factors, e.g., one or more of the following: Achaete-scute homolog 1 (Ascl1, e.g., UniProtKB -P50553 or Q02067), LIM homeobox transcription factor 1-alpha (Lmx1a, e.g., UniProtKB-Q8TE12 or Q9JKU8), Nurr1 (e.g., UniProtKB -P43354), preferably or alternatively Achaete-scute homolog 1 (Ascl1, e.g., UniProtKB-P50553 or Q02067), LIM homeobox transcription factor 1-alpha (Lmx1a, e.g., UniProtKB-Q8TE12 or Q9JKU8), Neurogenic differentiation factor 1 (Neurod1, e.g., UniProtKB-Q13562 or Q60867), a ncRNA (non-coding RNA, e.g., miRNA), miRNA218 (e.g., N R_029632, N R_029799. 1);
iii) for cell-replacement and/or transplantation;
iv) for somatic reprogramming of a cell; preferably said cell is selected from the group consisting of: an astrocyte, cardiomyocyte, adipocyte, muscle cell, osteoclast, osteoblast, osteocytes, a blood cell (e.g., white blood cell), a skin cell, a stem cell, further preferably said astrocyte is reprogrammed into a neuron;
v) for genome modification and/or gene therapy;
vi) for producing a neuron;
vii) as a medicament and/or in therapy;
viii) for treatment, amelioration, prophylaxis and/or diagnostics of a degenerative disease (e.g., neurodegenerative disease), cancer, cardiovascular disease, metabolic disease, monogenic disorder (e.g., single-gene associated disorder, e.g., Osteogenesis Imperfecta (OGI), Retinoblastoma (RB), Cystic Fibrosis, Thalassemia, Fragile X Syndrome (FXS), Hypophosphatemia, Hemophilia and Ichthyosis) and/or diabetes, preferably said neurodegenerative disease is selected from the group consisting of: Parkinson's disease, Parkinsonism, Parkinson-plus syndrome, Alzheimer's disease, Amyotrophic lateral sclerosis (ALS) and Huntington's disease;
ix) in a method according to any one of preceding claims;
x) for in vitro, in vivo or ex vivo use;
xi) in any combination of (i)-(x).


SEQUENCE LISTING (45 sequences)

SEQ ID NO: 1 - DNA, 2109 bp, Artificial Sequence
  DNA construct N-dCas9_N-Intein
  CDS (1)..(2109)
atgggacctaagaaaaagaggaaggtggcggccgctgactacaaggatgacgacgataaaggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcctgtcctacgagaccgaaatcctgaccgtggagtatgggctgctgcccatcggcaagattgtggagaagcggattgaatgcaccgtgtatagcgtggacaacaacggcaacatctacacccagcccgtggctcagtggcacgacaggggcgagcaggaggtgtttgagtattgtctggaggacggcagcctgattagagccaccaaagaccacaagttcatgaccgtggacgggcagatgctgcccattgacgagatttttgagcgggaacttgacctgatgcgggtggacaacctgcccaactga

SEQ ID NO: 2 - protein, 702 aa, Artificial Sequence
  Synthetic Construct
MGPKKKRKVAAADYKDDDDKGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 3 - DNA, 2790 bp, Artificial Sequence
  DNA construct C-dCas9_VP64_C-Intein
  CDS (1)..(2790)
atgataaagattgccaccagaaagtatctgggcaagcagaacgtgtatgacatcggcgtggagagagaccacaacttcgccctgaagaacggcttcatcgccagcaactgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcacccaagaagaagaggaaggtggctgcaggaggcggtggaagcgggcgcgccgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagtttga

SEQ ID NO: 4 - protein, 929 aa, Artificial Sequence
  Synthetic Construct
MIKIATRKYLGKQNVYDIGVERDHNFALKNGFIASNCFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVAAGGGGSGRADALDDFDLDMLGSDALDDFDLDMLGSDALDDFDLDMLGSDALDDFDLDMLINSRSSGSPKKKRKV

SEQ ID NO: 5 - DNA, 2052 bp, Artificial Sequence
  DNA construct CBh_flexed-GFP
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccccataacttcgtataaagtatcctatacgaagttatatcaaaataggaagaccaatgcttcaccatcgacccgaattgccaagcatcaccatcgacccataacttcgtataatgtatgctatacgaagttatactagggatcccccctcgagttttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcggtaccgaattctagtataacttcgtataggatactttatacgaagttatcattgggattcttcctattttgatccaagcatcaccatcgaccctctagtccagatctcaccatcgacccataacttcgtatagcatacattatacgaagttatgtccctcgaagaggttcgaattcgataataaaggaaatttattttcattgcaatagtgtgttggttttttgtgtg

SEQ ID NO: 6 - protein, 239 aa, Artificial Sequence
  Protein construct CBh_flexed-GFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 7 - DNA, 5793 bp, Artificial Sequence
  DNA construct HA_SpCas9(D10A,H840A)_VPR
  CDS (1)..(5793)
atgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagcgggccggccggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttga

SEQ ID NO: 8 - protein, 1930 aa, Artificial Sequence
  Synthetic Construct
MYPYDVPDYASPKKKRKVEASDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVEASGPAGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 9 - DNA, 306 bp, Artificial Sequence
  DNA construct N-Split-Intein
  CDS (1)..(306)
tgcctgtcctacgagaccgaaatcctgaccgtggagtatgggctgctgcccatcggcaagattgtggagaagcggattgaatgcaccgtgtatagcgtggacaacaacggcaacatctacacccagcccgtggctcagtggcacgacaggggcgagcaggaggtgtttgagtattgtctggaggacggcagcctgattagagccaccaaagaccacaagttcatgaccgtggacgggcagatgctgcccattgacgagatttttgagcgggaacttgacctgatgcgggtggacaacctgcccaac

SEQ ID NO: 10 - protein, 102 aa, Artificial Sequence
  Synthetic Construct
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 11 - DNA, 58 bp, Artificial Sequence
  DNA construct C-Split-Intein
  CDS (1)..(57)
catcggcgtggagagagaccacaacttcgccctgaagaacggcttcatcgccagcaac

SEQ ID NO: 12 - protein, 19 aa, Artificial Sequence
  Synthetic Construct
HRRGERPQLRPEERLHRQQ

SEQ ID NO: 13 - DNA, 390 bp, Artificial Sequence
  DNA construct MS2
  CDS (1)..(390)
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctac

SEQ ID NO: 14 - protein, 130 aa, Artificial Sequence
  Synthetic Construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 15 - DNA, 1032 bp, Artificial Sequence
  DNA construct MS2-p65
  CDS (1)..(1032)
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcgaccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcag

SEQ ID NO: 16 - protein, 344 aa, Artificial Sequence
  Synthetic Construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVATAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQ

SEQ ID NO: 17 - DNA, 1470 bp, Artificial Sequence
  DNA construct MS2-p65-HSF1
  CDS (1)..(1470)
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcgaccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctgtacatccgggtttgccaacgaactaggcccgcgacttatgggtaaataa

SEQ ID NO: 18 - protein, 489 aa, Artificial Sequence
  Synthetic Construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVATAGSPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSCTSGFANELGPRLMGK

SEQ ID NO: 19 - DNA, 39 bp, Artificial Sequence
  DNA construct VP16
  CDS (1)..(39)
gacgcattggacgattttgatctggatatgctgggaagt

SEQ ID NO: 20 - protein, 13 aa, Artificial Sequence
  Synthetic Construct
DALDDFDLDMLGS

SEQ ID NO: 21 - DNA, 10639 bp, Artificial Sequence
  DNA construct dCas9-SAM-P2A-VPR
ctagggcgcgccggattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagatccttaattaaccgtcttaagcctgtaacaaccggtacagttcgaataacttcgtatagcatacattatacgaagttataagcttgcatgcctgcaggtcggccgccacgaccggccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccacccggggcggccccgccgaccccgcacccgcccccgaggcccaccgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccataacttcgtatagcatacattatacgaagttataccgggccaccatagtcgcgagtagcttgggccagctaggccttgaccaaagttcctctggaattgcgatcgcgaagttcctattctctagaaagtataggaacttcgtgccgccaccatggactacaaggacgacgacgataaggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcggccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcccgaagttcctattctctagaaagtataggaacttcgggcagcggcgccaccaacttcagcctgctgaaacaggctggcgacgtcgaggaaaatcccgggccatgtacaatgtacccatacgatgttccagattacgcttcgccgaagaaaaagcgcaaggtcgaagcgtccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgccatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagcgggccggccggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttgaacgcgtaaatgattgcagatccactagttctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggctagc

SEQ ID NO: 22 - DNA, 4933 bp, Artificial Sequence
  DNA construct AAV-ALN-flexGFP
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggagccgctcgctgcagcagcggttttagagctaggccaacatgaggatcacccatgtctgcagggcctagcaagttaaaataaggctagtccgttatcaacttggccaacatgaggatcacccatgtctgcagggccaagtggcaccgagtcggtgctttttttcggtgcttttttttgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccagggggctgaatggagagtttgcagttttagagctaggccaacatgaggatcacccatgtctgcagggcctagcaagttaaaataaggctagtccgttatcaacttggccaacatgaggatcacccatgtctgcagggccaagtggcaccgagtcggtgctttttttcggtgcttttttttgatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgggggagcaaaggagtcgccttggttttagagctaggccaacatgaggatcacccatgtctgcagggcctagcaagttaaaataaggctagtccgttatcaacttggccaacatgaggatcacccatgtctgcagggccaagtggcaccgagtcggtgctttttttcggtgcttttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcggaatgcatccaagagtgaaccgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccaggggcggtgggtcattgtttccggttttagagctaggccaacatgaggatcacccatgtctgcagggcctagcaagttaaaataaggctagtccgttatcaacttggccaacatgaggatcacccatgtctgcagggccaagtggcaccgagtcggtgctttttttcggtgcttttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcggtgccagtgacgccggcctgggttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgagctgtactgagacgcacgtgcgccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccccataacttcgtataaagtatcctatacgaagttatatcaaaataggaagaccaatgcttcaccatcgacccgaattgccaagcatcaccatcgacccataacttcgtataatgtatgctatacgaagttatactagggatcccccctcgagttttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcggtaccgaattctagtataacttcgtataggatactttatacgaagttatcattgggattcttcctattttgatccaagcatcaccatcgaccctctagtccagatctcaccatcgacccataacttcgtatagcatacattatacgaagttatgtccctcgaagaggttcgaattcgataataaaggaaatttattttcattgcaatagtgtgttggttttttgtgtgcgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 23 - DNA, 3850 bp, Artificial Sequence
  DNA construct AAV-N-flex-dCas9aa1-573-N-intein
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaaggtcgggcaggaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaatacctcgggagccgctcgctgcagcagcggttttagagctaggccaacatgaggatcacccatgtctgcagggcctagcaagttaaaataaggctagtccgttatcaacttggccaacatgaggatcacccatgtctgcagggccaagtggcaccgagtcggtgcttttttttgagcgacctgtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggatccttaattaaataacttcgtatagcatacattatacgaagttatagtcataacttcgtatagtatacattatacgaagttatgttgggcaggttgtccacccgcatcaggtcaagttcccgctcaaaaatctcgtcaatgggcagcatctgcccgtccacggtcatgaacttgtggtctttggtggctctaatcaggctgccgtcctccagacaatactcaaacacctcctgctcgcccctgtcgtgccactgagccacgggctgggtgtagatgttgccgttgttgtccacgctatacacggtgcattcaatccgcttctccacaatcttgccgatgggcagcagcccatactccacggtcaggatttcggtctcgtaggacaggcactcgattttcttgaagtagtcctctttcagctgcttcacggtcactttccggttggtcttgaacagcaggtccacgatggcctttttctgctcgccgctcaggaaggcgggctttctcattccctcggtcacgtatttcactttggtcagctcgttatacacggtgaagtactcgtacagcaggctgtgcttgggcagcaccttctcgttgggcaggttcttatcgaagttggtcatccgctcgatgaagctctgggcggaagcgcccttgtccaccacttcctcgaagttccagggggtgatggtttcctcgctctttctggtcatccaggcgaatctgctgtttcccctggccagagggcccacgtagtaggggatgcggaaggtcaggatcttctcgatcttttcccggttgtccttcaggaatgggtaaaaatcttcctgccgccgcagaatggcgtgcagctctcccaggtggatctggtgggggatgctgccgttgtcgaaggtccgctgcttccgcagcaggtcctctctgttcagcttcacgagcagttcctcggtgccgtccatcttttccaggatgggcttgatgaacttgtagaactcttcctggctggctccgccgtcaatgtagccggcgtagccgttcttgctctggtcgaagaaaatctctttgtacttctcaggcagctgctgccgcacgagagctttcagcagggtcaggtcctggtggtgctcgtcgtatctcttgatcatagaggcgctcaggggggccttggtgatctcggtgttcactctcaggatgtcgctcagcaggatggcgtcggacaggttcttggcggccagaaacaggtcggcgtactggtcgccgatctgggccagcaggttgtccaggtcgtcgtcgtaggtgtccttgctcagctgcagtttggcatcctcggccaggtcgaagttgctcttgaagttgggggtcaggcccaggctcagggcaatcaggttgccgaacaggccattcttcttctcgccgggcagctgggcgatcagattttccagccgtctgctcttgctcagtctggcagacaggatggccttggcgtccacgccgctggcgttgatggggttttcctcgaacagctggttgtaggtctgcaccagctggatgaacagcttgtccacgtcgctgttgtcggggttcaggtcgccctcgatcaggaagtggccccggaacttgatcatgtgggccagggccagatagatcagccgcaggtcggccttgtcggtgctgtccaccagtttctttctcaggtggtagatggtggggtacttctcgtggtaggccacctcgtccacgatgttgccgaagatggggtgccgctcgtgcttcttatcctcttccaccaggaaggactcttccagtctgtggaagaagctgtcgtccaccttggccatctcgttgctgaagatctcttgcagatagcagatccggttcttccgtctggtgtatcttcttctggcggttctcttcagccgggtggcctcggctgtttcgccgctgtcgaacagcagggctccgatcaggttcttcttgatgctgtgccggtcggtgttgcccagcaccttgaatttcttgctgggcaccttgtactcgtcggtgatcacggcccagcccacagagttggtgccgatggccaggccgatgctgtacttcttgtcggctgctgggactccgtggatacctttatcgtcgtcatccttgtagtcagcggccgccaccttcctctttttcttaggtcccatggtggcggcataacttcgtataatgtatgctatacgaagttatagtcataacttcgtataatgtatactatacgaagttattgaacgcgtaataaaggaaatttattttcattgcaatagtgtgttggttttttgtgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 24 - DNA, 4406 bp, Artificial Sequence
  DNA construct AAV-C-dCas9aa574-1368-VP64-C- intein
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaaggtcgggcaggaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaatacctcggggctgaatggagagtttgcagttttagagctaggccaacatgaggatcacccatgtctgcagggcctagcaagttaaaataaggctagtccgttatcaacttggccaacatgaggatcacccatgtctgcagggccaagtggcaccgagtcggtgcttttttttgagcgacctgtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggatccttaattaataatacgactcactataggggccgccaccatgataaagattgccaccagaaagtatctgggcaagcagaacgtgtatgacatcggcgtggagagagaccacaacttcgccctgaagaacggcttcatcgccagcaactgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcacccaagaagaagaggaaggtggctgcaggaggcggtggaagcgggcgcgccgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagtttgaacgcgtaataaaggaaatttattttcattgcaatagtgtgttggttttttgtgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 25 - DNA, 4377 bp, Artificial Sequence
  DNA construct AAV-Lmx1a-Nr4a2-SAM
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggagcgatcgcgccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtggcgaccgctggatccccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgctccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcctgtacatccgggtttgccaacgaactaggcccgcgacttatgggtaaataaaataaaggaaatttattttcattgcaatagtgtgttggttttttgtgtggtcgacgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagcaaaggagtcgccttggttttagagctaggccaacatgaggatcacccatgtctgcagggcctagcaagttaaaataaggctagtccgttatcaacttggccaacatgaggatcacccatgtctgcagggccaagtggcaccgagtcggtgctttttttcggtgcttttttttgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccagaatgcatccaagagtgaaccgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttcggtgcttttttttgatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttggcggtgggtcattgtttccggttttagagctaggccaacatgaggatcacccatgtctgcagggcctagcaagttaaaataaggctagtccgttatcaacttggccaacatgaggatcacccatgtctgcagggccaagtggcaccgagtcggtgctttttttcggtgcttttttttctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctcggtgccagtgacgccggcctgggttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgagctgtactgagacgttaattaacgtgcagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 26 - DNA, 3255 bp, Artificial Sequence
  DNA construct AAV-flexGFP
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccccataacttcgtataaagtatcctatacgaagttatatcaaaataggaagaccaatgcttcaccatcgacccgaattgccaagcatcaccatcgacccataacttcgtataatgtatgctatacgaagttatactagggatcccccctcgagttttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcggtaccgaattctagtataacttcgtataggatactttatacgaagttatcattgggattcttcctattttgatccaagcatcaccatcgaccctctagtccagatctcaccatcgacccataacttcgtatagcatacattatacgaagttatgtccctcgaagaggttcgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctg

SEQ ID NO: 27 - protein, 1368 aa, Streptococcus pyogenes [sequence omitted]

SEQ ID NO: 28 - DNA, 23 bp, Artificial Sequence
  Ascl1-1
gggagccgctcgctgcagcagcg

SEQ ID NO: 29 - DNA, 22 bp, Artificial Sequence
  Ascl1-2
ggggctgaatggagagtttgca

SEQ ID NO: 30 - DNA, 21 bp, Artificial Sequence
  Lmx1a-1
gggagcaaaggagtcgccttg

SEQ ID NO: 31 - DNA, 21 bp, Artificial Sequence
  Lmx1a-2
gaatgcatccaagagtgaacc

SEQ ID NO: 32 - DNA, 21 bp, Artificial Sequence
  Nr4a2-1
ggcggtgggtcattgtttccg

SEQ ID NO: 33 - DNA, 21 bp, Artificial Sequence
  Nr4a2-2
gtgccagtgacgccggcctgg

SEQ ID NO: 34 - DNA, 21 bp, Artificial Sequence
  NeuroD1-1
ggttctgggaggggtgaatga

SEQ ID NO: 35 - DNA, 22 bp, Artificial Sequence
  NeuroD1-2
ggccatatggcgcatgccgggg

SEQ ID NO: 36 - DNA, 20 bp, Artificial Sequence
  Neurog2-1
ataagctggggagggagagc

SEQ ID NO: 37 - DNA, 20 bp, Artificial Sequence
  Neurog2-2
aaacaatcagatctgccccg

SEQ ID NO: 38 - DNA, 20 bp, Artificial Sequence
  PITX3-1
attcactttatggcaaccca

SEQ ID NO: 39 - DNA, 20 bp, Artificial Sequence
  PITX3-2
gctagcctgggagagcccag

SEQ ID NO: 40 - DNA, 21 bp, Artificial Sequence
  FoxA2-1
gaaagtaaccttgaaacaccg

SEQ ID NO: 41 - DNA, 21 bp, Artificial Sequence
  FoxA2-2
gggtagccagaaagaggactg

SEQ ID NO: 42 - DNA, 20 bp, Artificial Sequence
  MyoD1-1
ccaataggagtgtagtaggg

SEQ ID NO: 43 - DNA, 20 bp, Artificial Sequence
  MyoD1-2
gagagactggcagccatacg

SEQ ID NO: 44 - DNA, 20 bp, Artificial Sequence
  Pou5f1-1
atctgcctgtgtcttccaga

SEQ ID NO: 45 - DNA, 20 bp, Artificial Sequence
  Pou5f1-2
tgtccggtgacccaaggcag